SEQUENCE LISTING (133 sequences)

SEQ ID NO: 1 - protein, 14 aa, Artificial
  Description of Artificial Sequence Binding peptide
XXXXXXXXXXXXXX

SEQ ID NO: 2 - protein, 14 aa, Artificial
  Description of Artificial Sequence Binding peptide
RARLQRRSARLSAK

SEQ ID NO: 3 - protein, 14 aa, Artificial
  Description of Artificial Sequence Binding peptide
KARLQRRSARLSAK

SEQ ID NO: 4 - protein, 14 aa, Artificial
  Description of Artificial Sequence Binding peptide
VARLQRRSARLSAK

SEQ ID NO: 5 - protein, 14 aa, Artificial
  Description of Artificial Sequence Binding peptide
HARLQRRSARLSAK

SEQ ID NO: 6 - protein, 14 aa, Artificial
  Description of Artificial Sequence Binding peptide
GARLQRRSARLSAK

SEQ ID NO: 7 - protein, 14 aa, Artificial
  Description of Artificial Sequence Binding peptide
RAKLQRRSARLSAK

SEQ ID NO: 8 - protein, 14 aa, Artificial
  Description of Artificial Sequence Binding peptide
HAHLQRRSARLSAK

SEQ ID NO: 9 - protein, 14 aa, Artificial
  Description of Artificial Sequence Binding peptide
RARLQRRFARLFAK

SEQ ID NO: 10 - protein, 14 aa, Artificial
  Description of Artificial Sequence Binding peptide
KARLQRRFARLFAK

SEQ ID NO: 11 - protein, 14 aa, Artificial
  Description of Artificial Sequence Binding peptide
VARLQRRFARLFAK

SEQ ID NO: 12 - protein, 14 aa, Artificial
  Description of Artificial Sequence Binding peptide
GARLQRRFARLFAK

SEQ ID NO: 13 - protein, 14 aa, Artificial
  Description of Artificial Sequence Binding peptide
HARLQRRFARLFAK

SEQ ID NO: 14 - protein, 14 aa, Artificial
  Description of Artificial Sequence Binding peptide
HAHLQRRFARLFAK

SEQ ID NO: 15 - protein, 14 aa, Artificial
  Description of Artificial Sequence Binding peptide
RAKLQRRFARLFAK

SEQ ID NO: 16 - protein, 14 aa, Artificial
  Description of Artificial Sequence Binding peptide
RARLQRRLARLLAK

SEQ ID NO: 17 - protein, 14 aa, Artificial
  Description of Artificial Sequence Binding peptide
KARLQRRLARLLAK

SEQ ID NO: 18 - protein, 14 aa, Artificial
  Description of Artificial Sequence Binding peptide
VARLQRRLARLLAK

SEQ ID NO: 19 - protein, 14 aa, Artificial
  Description of Artificial Sequence Binding peptide
GARLQRRLARLLAK

SEQ ID NO: 20 - protein, 14 aa, Artificial
  Description of Artificial Sequence Binding peptide
HARLQRRLARLLAK

SEQ ID NO: 21 - protein, 14 aa, Artificial
  Description of Artificial Sequence Binding peptide
RAKLQRRLARLLAK

SEQ ID NO: 22 - protein, 14 aa, Artificial
  Description of Artificial Sequence Binding peptide
HAHLQRRLARLLAK

SEQ ID NO: 23 - protein, 13 aa, Artificial
  Description of Artificial Sequence Binding peptide
RARLQRRSARLSA

SEQ ID NO: 24 - protein, 13 aa, Artificial
  Description of Artificial Sequence Binding peptide
KARLQRRSARLSA

SEQ ID NO: 25 - protein, 13 aa, Artificial
  Description of Artificial Sequence Binding peptide
VARLQRRSARLSA

SEQ ID NO: 26 - protein, 13 aa, Artificial
  Description of Artificial Sequence Binding peptide
HARLQRRSARLSA

SEQ ID NO: 27 - protein, 13 aa, Artificial
  Description of Artificial Sequence Binding peptide
GARLQRRSARLSA

SEQ ID NO: 28 - protein, 13 aa, Artificial
  Description of Artificial Sequence Binding peptide
RAKLQRRSARLSA

SEQ ID NO: 29 - protein, 13 aa, Artificial
  Description of Artificial Sequence Binding peptide
HAHLQRRSARLSA

SEQ ID NO: 30 - protein, 13 aa, Artificial
  Description of Artificial Sequence Binding peptide
RARLQRRFARLFA

SEQ ID NO: 31 - protein, 13 aa, Artificial
  Description of Artificial Sequence Binding peptide
KARLQRRFARLFA

SEQ ID NO: 32 - protein, 13 aa, Artificial
  Description of Artificial Sequence Binding peptide
VARLQRRFARLFA

SEQ ID NO: 33 - protein, 13 aa, Artificial
  Description of Artificial Sequence Binding peptide
GARLQRRFARLFA

SEQ ID NO: 34 - protein, 13 aa, Artificial
  Description of Artificial Sequence Binding peptide
HARLQRRFARLFA

SEQ ID NO: 35 - protein, 13 aa, Artificial
  Description of Artificial Sequence Binding peptide
HAHLQRRFARLFA

SEQ ID NO: 36 - protein, 13 aa, Artificial
  Description of Artificial Sequence Binding peptide
RAKLQRRFARLFA

SEQ ID NO: 37 - protein, 13 aa, Artificial
  Description of Artificial Sequence Binding peptide
RARLQRRLARLLA

SEQ ID NO: 38 - protein, 13 aa, Artificial
  Description of Artificial Sequence Binding peptide
KARLQRRLARLLA

SEQ ID NO: 39 - protein, 13 aa, Artificial
  Description of Artificial Sequence Binding peptide
VARLQRRLARLLA

SEQ ID NO: 40 - protein, 13 aa, Artificial
  Description of Artificial Sequence Binding peptide
GARLQRRLARLLA

SEQ ID NO: 41 - protein, 13 aa, Artificial
  Description of Artificial Sequence Binding peptide
HARLQRRLARLLA

SEQ ID NO: 42 - protein, 13 aa, Artificial
  Description of Artificial Sequence Binding peptide
RAKLQRRLARLLA

SEQ ID NO: 43 - protein, 13 aa, Artificial
  Description of Artificial Sequence Binding peptide
HAHLQRRLARLLA

SEQ ID NO: 44 - protein, 10 aa, Artificial
  Description of Artificial Sequence Binding peptide
RARLQRRSAR

SEQ ID NO: 45 - protein, 10 aa, Artificial
  Description of Artificial Sequence Binding peptide
KARLQRRSAR

SEQ ID NO: 46 - protein, 10 aa, Artificial
  Description of Artificial Sequence Binding peptide
VARLQRRSAR

SEQ ID NO: 47 - protein, 11 aa, Artificial
  Description of Artificial Sequence Binding peptide
HARLQRRSARL

SEQ ID NO: 48 - protein, 10 aa, Artificial
  Description of Artificial Sequence Binding peptide
GARLQRRSAR

SEQ ID NO: 49 - protein, 10 aa, Artificial
  Description of Artificial Sequence Binding peptide
RAKLQRRSAR

SEQ ID NO: 50 - protein, 10 aa, Artificial
  Description of Artificial Sequence Binding peptide
HAHLQRRSAR

SEQ ID NO: 51 - protein, 10 aa, Artificial
  Description of Artificial Sequence Binding peptide
RARLQRRFAR

SEQ ID NO: 52 - protein, 10 aa, Artificial
  Description of Artificial Sequence Binding peptide
KARLQRRFAR

SEQ ID NO: 53 - protein, 10 aa, Artificial
  Description of Artificial Sequence Binding peptide
VARLQRRFAR

SEQ ID NO: 54 - protein, 13 aa, Artificial
  Description of Artificial Sequence Binding peptide
GARLQRRFARLFA

SEQ ID NO: 55 - protein, 10 aa, Artificial
  Description of Artificial Sequence Binding peptide
HARLQRRFAR

SEQ ID NO: 56 - protein, 10 aa, Artificial
  Description of Artificial Sequence Binding peptide
HAHLQRRFAR

SEQ ID NO: 57 - protein, 10 aa, Artificial
  Description of Artificial Sequence Binding peptide
RAKLQRRFAR

SEQ ID NO: 58 - protein, 10 aa, Artificial
  Description of Artificial Sequence Binding peptide
RARLQRRLAR

SEQ ID NO: 59 - protein, 10 aa, Artificial
  Description of Artificial Sequence Binding peptide
KARLQRRLAR

SEQ ID NO: 60 - protein, 10 aa, Artificial
  Description of Artificial Sequence Binding peptide
VARLQRRLAR

SEQ ID NO: 61 - protein, 10 aa, Artificial
  Description of Artificial Sequence Binding peptide
GARLQRRLAR

SEQ ID NO: 62 - protein, 10 aa, Artificial
  Description of Artificial Sequence Binding peptide
HARLQRRLAR

SEQ ID NO: 63 - protein, 10 aa, Artificial
  Description of Artificial Sequence Binding peptide
RAKLQRRLAR

SEQ ID NO: 64 - protein, 10 aa, Artificial
  Description of Artificial Sequence Binding peptide
HAHLQRRLAR

SEQ ID NO: 65 - protein, 14 aa, Artificial
  Description of Artificial Sequence Binding peptide
RGRLQRRSARLSAK

SEQ ID NO: 66 - protein, 14 aa, Artificial
  Description of Artificial Sequence Binding peptide
RVRLQRRSARLSAK

SEQ ID NO: 67 - protein, 13 aa, Artificial
  Description of Artificial Sequence Binding peptide
RGRLQRRSARLSA

SEQ ID NO: 68 - protein, 13 aa, Artificial
  Description of Artificial Sequence Binding peptide
RVRLQRRSARLSA

SEQ ID NO: 69 - protein, 12 aa, Artificial
  Description of Artificial Sequence Binding peptide
RGRLQRRSARLS

SEQ ID NO: 70 - protein, 12 aa, Artificial
  Description of Artificial Sequence Binding peptide
RVRLQRRSARLS

SEQ ID NO: 71 - protein, 11 aa, Artificial
  Description of Artificial Sequence Binding peptide
RGRLQRRSARL

SEQ ID NO: 72 - protein, 11 aa, Artificial
  Description of Artificial Sequence Binding peptide
RVRLQRRSARL

SEQ ID NO: 73 - protein, 10 aa, Artificial
  Description of Artificial Sequence Binding peptide
RGRLQRRSAR

SEQ ID NO: 74 - protein, 10 aa, Artificial
  Description of Artificial Sequence Binding peptide
RVRLQRRSAR

SEQ ID NO: 75 - protein, 14 aa, Artificial
  Description of Artificial Sequence Lytic peptide
KFAKFAKKFAKFAK

SEQ ID NO: 76 - protein, 15 aa, Artificial
  Description of Artificial Sequence Lytic peptide
KFAKFAKKFAKFAKK

SEQ ID NO: 77 - protein, 16 aa, Artificial
  Description of Artificial Sequence Lytic peptide
KFAKFAKKFAKFAKKF

SEQ ID NO: 78 - protein, 17 aa, Artificial
  Description of Artificial Sequence Lytic peptide
KFAKFAKKFAKFAKKFA

SEQ ID NO: 79 - protein, 18 aa, Artificial
  Description of Artificial Sequence Lytic peptide
KFAKFAKKFAKFAKKFAK

SEQ ID NO: 80 - protein, 19 aa, Artificial
  Description of Artificial Sequence Lytic peptide
KFAKFAKKFAKFAKKFAKF

SEQ ID NO: 81 - protein, 20 aa, Artificial
  Description of Artificial Sequence Lytic peptide
KFAKFAKKFAKFAKKFAKFA

SEQ ID NO: 82 - protein, 21 aa, Artificial
  Description of Artificial Sequence Lytic peptide
KFAKFAKKFAKFAKKFAKFAK

SEQ ID NO: 83 - protein, 14 aa, Artificial
  Description of Artificial Sequence Lytic peptide
KFAKFAKKFAKFAK

SEQ ID NO: 84 - protein, 15 aa, Artificial
  Description of Artificial Sequence Lytic peptide
KFAKFAKKFAKFAKK

SEQ ID NO: 85 - protein, 16 aa, Artificial
  Description of Artificial Sequence Lytic peptide
KFAKFAKKFAKFAKKF

SEQ ID NO: 86 - protein, 17 aa, Artificial
  Description of Artificial Sequence Lytic peptide
KFAKFAKKFAKFAKKFA

SEQ ID NO: 87 - protein, 18 aa, Artificial
  Description of Artificial Sequence Lytic peptide
KFAKFAKKFAKFAKKFAK

SEQ ID NO: 88 - protein, 19 aa, Artificial
  Description of Artificial Sequence Lytic peptide
KFAKFAKKFAKFAKKFAKF

SEQ ID NO: 89 - protein, 20 aa, Artificial
  Description of Artificial Sequence Lytic peptide
KFAKFAKKFAKFAKKFAKFA

SEQ ID NO: 90 - protein, 21 aa, Artificial
  Description of Artificial Sequence Lytic peptide
KFAKFAKKFAKFAKKFAKFAK

SEQ ID NO: 91 - protein, 23 aa, Artificial
  Description of Artificial Sequence Lytic peptide
FALALKALKKALKKLKKALKKAL

SEQ ID NO: 92 - protein, 23 aa, Artificial
  Description of Artificial Sequence Lytic peptide
FAFAFKAFKKAFKKFKKAFKKAF

SEQ ID NO: 93 - protein, 23 aa, Artificial
  Description of Artificial Sequence Lytic peptide
FAKKFAKKFKKFAKKFAKFAFAF

SEQ ID NO: 94 - protein, 27 aa, Artificial
  Description of Artificial Sequence Lytic peptide
KRKRAVKRVGRRLKKLARKIARLGVAF

SEQ ID NO: 95 - protein, 37 aa, Artificial
  Description of Artificial Sequence Lytic peptide
KRKRAVKRVGRRLKKLARKIARLGVAKLAGLRAVLKF

SEQ ID NO: 96 - protein, 31 aa, Artificial
  Description of Artificial Sequence Binding peptide
KDEPQRRSARLSAKPAPPKPEPKPKKAPAKK

SEQ ID NO: 97 - protein, 14 aa, Artificial
  Description of Artificial Sequence Binding peptide
KDEPQRRSARLSAK

SEQ ID NO: 98 - protein, 4 aa, Artificial
  Description of Artificial Sequence Binding peptide
KDEP

SEQ ID NO: 99 - protein, 8 aa, Artificial
  Description of Artificial Sequence Binding peptide
RARLRARL

SEQ ID NO: 100 - protein, 14 aa, Artificial
  Description of Artificial Sequence Binding peptide
RARLQRRSARLSAK

SEQ ID NO: 101 - protein, 11 aa, Artificial
  Description of Artificial Sequence Binding peptide
RARLQRRSARL

SEQ ID NO: 102 - protein, 12 aa, Artificial
  Description of Artificial Sequence Binding peptide
RARLQRRSARLS

SEQ ID NO: 103 - protein, 14 aa, Artificial
  Description of Artificial Sequence Binding peptide
HARLQRRSARLSAK

SEQ ID NO: 104 - protein, 14 aa, Artificial
  Description of Artificial Sequence Binding peptide
RVRLQRRSARLSAK

SEQ ID NO: 105 - protein, 14 aa, Artificial
  Description of Artificial Sequence Binding peptide
HAHLQRRSARLSAK

SEQ ID NO: 106 - protein, 18 aa, Artificial
  Description of Artificial Sequence Lytic peptide
KFAKFAKKFAKFAKKFAK

SEQ ID NO: 107 - protein, 32 aa, Artificial
  Description of Artificial Sequence Lytic fusion peptide
KFAKFAKKFAKFAKKFAKKDEPQRRSARLSAK

SEQ ID NO: 108 - protein, 32 aa, Artificial
  Description of Artificial Sequence Lytic fusion peptide
KFAKFAKKFAKFAKKFAKRARLQRRSARLSAK

SEQ ID NO: 109 - protein, 31 aa, Artificial; identical to SEQ ID NO: 96
  Description of Artificial Sequence Binding peptide

SEQ ID NO: 110 - protein, 14 aa, Artificial
  Description of Artificial Sequence Binding peptide
KDEPQRRSARLSAK

SEQ ID NO: 111 - protein, 14 aa, Artificial
  Description of Artificial Sequence Binding peptide
RARLQRRSARLSAK

SEQ ID NO: 112 - protein, 11 aa, Artificial
  Description of Artificial Sequence Binding peptide
RARLQRRSARL

SEQ ID NO: 113 - protein, 12 aa, Artificial
  Description of Artificial Sequence Binding peptide
RARLQRRSARLS

SEQ ID NO: 114 - protein, 14 aa, Artificial
  Description of Artificial Sequence Binding peptide
HARLQRRSARLSAK

SEQ ID NO: 115 - protein, 14 aa, Artificial
  Description of Artificial Sequence Binding peptide
RVRLQRRSARLSAK

SEQ ID NO: 116 - protein, 14 aa, Artificial
  Description of Artificial Sequence Binding peptide
HAHLQRRSARLSAK

SEQ ID NO: 117 - protein, 31 aa, Artificial; identical to SEQ ID NO: 96
  Description of Artificial Sequence Lytic fusion peptide

SEQ ID NO: 118 - protein, 14 aa, Artificial
  Description of Artificial Sequence Lytic fusion peptide
KDEPQRRSARLSAK

SEQ ID NO: 119 - protein, 5 aa, Artificial
  Description of Artificial Sequence Linker
GSGGS

SEQ ID NO: 120 - protein, 5 aa, Artificial
  Description of Artificial Sequence Linker
ASAAS

SEQ ID NO: 121 - protein, 6 aa, Artificial
  Description of Artificial Sequence Linker
GSGRSA

SEQ ID NO: 122 - protein, 6 aa, Artificial
  Description of Artificial Sequence Linker
RVRRSV

SEQ ID NO: 123 - protein, 32 aa, Artificial
  Description of Artificial Sequence Lytic fusion peptide
KFAKFAKKFAKFAKKFAKVARLQRRFARLFAK

SEQ ID NO: 124 - protein, 32 aa, Artificial Sequence; identical to SEQ ID NO: 123
  Description of Artificial Sequence Lytic fusion peptide

SEQ ID NO: 125 - protein, 27 aa, Artificial
  Description of Artificial Sequence Lytic fusion peptide
KFAKFAKKFAKFAKKFAKVARLQRRFA

SEQ ID NO: 126 - protein, 26 aa, Artificial
  Description of Artificial Sequence Lytic fusion peptide
KFAKFAKKFAKFAKKFAKRARLQRRF

SEQ ID NO: 127 - protein, 25 aa, Artificial
  Description of Artificial Sequence Lytic fusion peptide
KFAKFAKKFAKFAKKFAKRARLQRR

SEQ ID NO: 128 - protein, 30 aa, Artificial
  Description of Artificial Sequence Lytic fusion peptide
KFAKFAKKFAKFAKKFAKRLQRRFARLFAK

SEQ ID NO: 129 - protein, 30 aa, Artificial
  Description of Artificial Sequence Lytic fusion peptide
KFAKFAKKFAKFAKKFAKRLQRRSARLSAK

SEQ ID NO: 130 - protein, 25 aa, Artificial
  Description of Artificial Sequence Binding peptide
KFAKFAKKFAKFAKKFAKFARLFAK

SEQ ID NO: 131 - protein, 25 aa, Artificial
  Description of Artificial Sequence Lytic fusion peptide
KFAKFAKKFAKFAKKFAKSARLSAK

SEQ ID NO: 132 - protein, 23 aa, Artificial
  Description of Artificial Sequence Lytic fusion peptide
KFAKFAKKFAKFAKKFAKRARLQ

SEQ ID NO: 133 - protein, 6 aa, Artificial
  Description of Artificial Sequence Linker
NRVRRS

(19) United States
(12) Patent Application Publication
(10) Pub. No.: US 2011/0124564 A1
(43) Pub. Date: May 26, 2011

(54) NUCLEOLIN-BINDING PEPTIDES, NUCLEOLIN- BINDING LYTIC PEPTIDES, FUSION CONSTRUCTS AND METHODS OF MAKING AND USING SAME

(75) Inventors: Hector ALILA, Baton Rouge, LA (US); Carola LEUSCHNER, Baton Rouge, LA (US)

(73) Assignee: ESPERANCE PHARMACEUTICALS, INC., Baton Rouge, LA (US)

(21) Appl. No.: 12/868,353

(22) Filed: Aug. 25, 2010

Related U.S. Application Data
(60) Provisional application No. 61/236,754, filed on Aug. 25, 2009.
(60) Provisional application No. 61/352,555, filed on Jun. 8, 2010.

(51) Int. Cl. A61K 38/10; C07K 7/06; C07K 7/08; C07K 14/00; C07K 19/00; C07K 16/18; A61K 38/08; A61K 38/02; C12N 5/071; A61P 35/00

(52) U.S. Cl. 514/13.3; 530/328; 530/327; 530/326; 530/325; 530/324; 530/387.9; 530/387.3; 514/21.6; 514/21.5; 435/375; 514/19.2; 514/19.3

(57) ABSTRACT

The invention relates to nucleolin binding peptides, nucleolin binding peptides and anti-nucleolin antibody conjugates with cytotoxic activity, fusion constructs, methods of using nucleolin binding peptides and antibodies and fusion constructs thereof, and methods of treating various disorders, undesirable conditions and diseases treatable with nucleolin binding peptides and fusion constructs, such as undesirable or aberrant cell proliferation (hyperproliferation) or hyperproliferative disorders, including tumors, cancers, neoplasia and malignancies, angiogenesis related or dependent diseases, and inflammatory diseases and inflammation.

### RELATED APPLICATIONS

This application claims priority to application Ser. No. 61/352,555, filed Jun. 8, 2010, and application Ser. No. 61/236,754, filed Aug. 25, 2009, each of which applications are expressly incorporated herein by reference in their entirety.

### TECHNICAL FIELD

The invention relates to nucleolin targeting/binding peptides, nucleolin targeting/binding lytic peptides, nucleolin targeting/binding lytic peptide and antibody fusion constructs, and methods of using nucleolin targeting/binding peptides, nucleolin targeting/binding lytic peptides, and fusion constructs such as treating undesirable or aberrant cell proliferation or hyperproliferative disorders, including non-metastatic and metastatic neoplasias, cancers, tumors and malignancies.

### INTRODUCTION

The need to develop new therapeutics for treatment of primary tumors and metastases is clearly evident when the five year survival rate of cancer patients is considered: Only 10-40% for patients with lung, colorectal, breast and prostate cancer survive if diagnosed with distant metastatic disease.

Traditional treatment methods such as chemotherapy or radiation therapies can cause substantial damage to normal cells and tissues and cause undesirable side-effects to the patients. Thus, there is also a need for targeted killing of cancer cells.

Tumor growth is supported by the formation of new blood vessels, a process known as angiogenesis. Tumor angiogenesis is the proliferation of a network of blood vessels that penetrates into cancerous growths, supplying nutrients and oxygen and removing waste products. Angiogenesis is a critical event in cancer metastasis. Thus, in addition to kill cancer cells directly, inhibition of angiogenesis can be a powerful method to treat cancer.

Emerging evidence suggests that enhanced expression of nucleolin is implicated in growth of tumor cells and angiogenesis (Storck S, et al., Biochemistry 29:9708 (2007); Christian et al., J Cell Biol. 163:871 (2003)). Nucleolin is over-expressed in the nucleus, cytoplasm and cell surface of cancer and endothelial cells. In the nucleus, nucleolin is believed to control many aspects of DNA and RNA metabolism; and in the cytoplasm it shuttles proteins into the nucleus and ribosomes to regulate important post-transcriptional events. On the cell surface it serves as a binding protein for variety of ligands implicated in cell proliferation, differentiation, adhesion, mitogenesis and angiogenesis. Because nucleolin is expressed on the surface of cancer and endothelial cells, it is a potential target for developing new drugs to treat cancer.

Nucleolin protein contains three structural domains: N-terminal region containing acidic residues; central domain containing oligonucleotide (such as RNA) binding sites; and C-terminal domain containing nine repeats of arginine-glycine-glycine (RGG). Molecules that bind to central domain and C-terminal domains have been shown to have anti-cancer effects. For example, DNA oligonucleotides (also known as aptamers, Soundararajan et al., Cancer Research 68:2358 (2008)) which bind to the central domain of nucleolin and a pseudopeptide known as HB-19, which binds to the C-terminal domain of nucleolin have been reported to inhibit cancer growth in patients with renal cancer and acute myeloid leukemia and HB-19 inhibited tumor growth and angiogenesis in mouse models of cancer (Destouches et al., PLoS ONE 3:e2518 (2008)).

### SUMMARY

The invention is based, at least in part, on peptides that bind to nucleolin alone or in a fusion construct, such as conjugated to a cytotoxic domain (e.g., lytic domain). Such peptides that bind to nucleolin, or fusion constructs that include such a peptide and a cytotoxic domain (e.g., lytic domain), can selectively bind to cells that express nucleolin, including undesirable or aberrant proliferating cells or hyperproliferating cells, such as non-metastatic and metastatic neoplasias, cancers, tumors and malignancies. Other cells that express nucleolin can also be targeted, such as endothelial cells that express nucleolin. The invention therefore provides peptides that bind to nucleolin, that are optionally cytotoxic to cells expressing nucleolin, and that have cytotoxic activity alone, or in a combination with a cytotoxic moiety or domain (e.g., lytic peptide) such as in a fusion construct.

Peptides of the invention include sequences based upon or derived from a part of high mobility group nucleosomal-binding domain 2 (HMGN2) sequence, which binds to nucleolin. Peptides of the invention include peptides that cause disruption of the cell membrane which causes toxicity and results in cell death, or prevention, inhibition or reduction in cell growth or proliferation. The peptides include a nucleolin binding moiety that bind to cells expressing nucleolin and that can optionally target such cells for lysis or death when possessing cytotoxicity. Certain nucleolin binding peptides of the invention therefore have cell toxicity alone, and need not include or be linked to a cytotoxic moiety or domain (e.g., lytic domain sequence). In such embodiments, the nucleolin binding peptides need not have a separate or additional cytotoxic moiety or domain in order to be toxic towards cells, and to stimulate, promote or induce cell lysis or death, or prevent, inhibit or reduce cell growth or proliferation.

In other embodiments, such peptides bind to nucleolin but may have little or no cytotoxic activity without a separate or additional cytotoxic moiety or domain (e.g., lytic domain sequence) that is toxic towards cells, and that stimulates, promotes or induces cell lysis or death, or prevents, inhibits or reduces cell growth or proliferation. In either case, peptides that bind to nucleolin having or not having cytotoxicity can be linked to a second separate or additional cytotoxic moiety, domain or region (e.g., lytic domain sequence) that stimulates, promotes or induces cell lysis or death, or prevents, inhibits or reduces cell growth or proliferation.

Peptides of the invention are useful in detecting nucleolin, or nucleolin expressing cells, tissue or organs (e.g., in a sample). As a number of cells, such as hyperproliferating, non-metastatic and metastatic neoplastic, cancer, tumor and malignant, and endothelial cells, express nucleolin, such peptides can be used to detect, diagnose or screen for the presence of such cells, tissue or organs (e.g., in a sample). Nucleolin binding peptides of the invention that exhibit cell cytotoxicity (i.e. are “lytic”) are useful for stimulating, inducing or promoting cell lysis or death expressing nucleolin, or preventing, inhibiting or reducing growth or proliferation of cells that express nucleolin. As a number of cells including hyperproliferating cells, non-metastatic and metastatic neoplastic, cancer, tumor and malignant cells, and endothelial cells that participate in angiogenesis express nucleolin, such peptides can be used to target such cells for lysis or death, thereby leading to a reduction or decrease of target cells and consequent treatment of any disease, disorder or undesirable condition associated with or caused by the presence of such cells.

Invention peptides that bind to nucleolin include a continuous amino acid sequence with the general formula, X1-X2-X3-X4-X5-X6-X7-X8-X9-X10-X11-X12-X13-X14, wherein: X1 is R, H, G, K or V; X2 is A, G or V; X3 is R, K or H; X4 is L; X5 is Q; X6 is R; X7 is R; X8 is S, F or L; X9 is A; X10 is R; X11 is L or nothing (absent); X12 is S, F or L or nothing (absent); X13 is A or nothing (absent); X14 is K or nothing (absent). In particular embodiments, a peptide includes the sequence: RARLQRRSARLSAK, KARLQRRSARLSAK, VARLQRRSARLSAK, HARLQRRSARLSAK, GARLQRRSARLSAK, RAKLQRRSARLSAK, HAHLQRRSARLSAK, RARLQRRFARLFAK, KARLQRRFARLFAK, VARLQRRFARLFAK, GARLQRRFARLFAK, HARLQRRFARLFAK, HAHLQRRFARLFAK, RAKLQRRFARLFAK, RARLQRRLARLLAK, KARLQRRLARLLAK, VARLQRRLARLLAK, GARLQRRLARLLAK, HARLQRRLARLLAK, RAKLQRRLARLLAK, HAHLQRRLARLLAK, RARLQRRSARLSA, KARLQRRSARLSA, VARLQRRSARLSA, HARLQRRSARLSA, GARLQRRSARLSA, RAKLQRRSARLSA, HAHLQRRSARLSA, RARLQRRFARLFA, KARLQRRFARLFA, VARLQRRFARLFA, GARLQRRFARLFA, HARLQRRFARLFA, HAHLQRRFARLFA, RAKLQRRFARLFA, RARLQRRLARLLA, KARLQRRLARLLA, VARLQRRLARLLA, GARLQRRLARLLA, HARLQRRLARLLA, RAKLQRRLARLLA, HAHLQRRLARLLA, RARLQRRSAR, KARLQRRSAR, VARLQRRSAR, HARLQRRSARL, GARLQRRSAR, RAKLQRRSAR, HAHLQRRSAR, RARLQRRFAR, KARLQRRFAR, VARLQRRFAR, GARLQRRFARLFA, HARLQRRFAR, HAHLQRRFAR, RAKLQRRFAR, RARLQRRLAR, KARLQRRLAR, VARLQRRLAR, GARLQRRLAR, HARLQRRLAR, RAKLQRRLAR, HABLQRRLAR, RGRLQRRSARLSAK, RVRLQRRSARLSAK, RGRLQRRSARLSA, RVRLQRRSARLSA, RGRLQRRSARLS, RVRLQRRSARLS, RGRLQRRSARL, RVRLQRRSARL, RGRLQRRSAR, or RVRLQRRSAR. In further embodiments, a peptide includes the sequence: RARLQRRSARLSAK, KARLQRRSARLSAK, VARLQRRSARLSAK, HARLQRRSARLSAK, GARLQRRSARLSAK, RAKLQRRSARLSAK, HAHLQRRSARLSAK, RARLQRRFARLFAK, KARLQRRFARLFAK, VARLQRRFARLFAK, GARLQRRFARLFAK, HARLQRRFARLFAK, HAHLQRRFARLFAK, RAKLQRRFARLFAK, RARLQRRLARLLAK, KARLQRRLARLLAK, VARLQRRLARLLAK, GARLQRRLARLLAK, HARLQRRLARLLAK, RAKLQRRLARLLAK, HAHLQRRLARLLAK, RARLQRRSARLSA, KARLQRRSARLSA, VARLQRRSARLSA, HARLQRRSARLSA, GARLQRRSARLSA, RAKLQRRSARLSA, HARLQRRSARLSA, RARLQRRFARLFA, KARLQRRFARLFA, VARLQRRFARLFA, GARLQRRFARLFA, HARLQRRFARLFA, HAHLQRRFARLFA, RAKLQRRFARLFA, RARLQRRLARLLA, KARLQRRLARLLA, VARLQRRLARLLA, GARLQRRLARLLA, HARLQRRLARLLA, RAKLQRRLARLLA, HAHLQRRLARLLA, RARLQRRSAR, KARLQRRSAR, VARLQRRSAR, HARLQRRSARL, GARLQRRSAR, RAKLQRRSAR, HAHLQRRSAR, RARLQRRFAR, KARLQRRFAR, VARLQRRFAR, GARLQRRFARLFA, HARLQRRFAR, HAHLQRRFAR, RAKLQRRFAR, RARLQRRLAR, KARLQRRLAR, VARLQRRLAR, GARLQRRLAR, HARLQRRLAR, RAKLQRRLAR, HAHLQRRLAR, RGRLQRRSARLSAK, RVRLQRRSARLSAK, RGRLQRRSARLSA, RVRLQRRSARLSA, RGRLQRRSARLS, RVRLQRRSARLS, RGRLQRRSARL, RVRLQRRSARL, RGRLQRRSAR, or RVRLQRRSAR, with one or more amino acid substitutions (e.g., a conservative or non-conservative substitution), and the peptide binds to nucleolin.

Invention peptides that bind to nucleolin also include antibodies and fragments/subsequences of antibodies that bind to nucleolin (cell surface expressed nucleolin, etc.). Such antibodies and fragments/subsequences that bind to nucleolin can be conjugated or linked to one or more peptides that target such cells for lysis or death or cause disruption of the cell membrane, such as invention peptides and/or lytic peptide domains to form a fusion construct. Selective toxicity towards nucleolin expressing cells is therefore achieved.

Specific non-limiting classes of antibodies include monoclonal, polyclonal, mammalian, primatized, humanized and human antibodies, and chimeric antibodies. Specific non-limiting examples of antibody fragments include Fab, Fab′, F(ab′)₂, Fv, Fd, single-chain Fv (scFv), disulfide-linked Fvs (sdFv), V_{L}, V_{H}, Camel Ig, V-NAR, VHH, trispecific (Fab₃), bispecific (Fab₂), diabody ((V_{L}-V_{H})₂ or (V_{H}-V_{L})₂), triabody (trivalent), tetrabody (tetravalent), minibody ((scF_{V}-C_{H}3)₂), bispecific single-chain Fv (Bis-scFv), IgGdeltaCH2, scFv-Fc or (scFv)₂-Fc and CH domains engineered to bind to nucleolin, as well as affibody, aptamer, avimer and nanobodies.

Peptides of the invention include sequences in which all or a part of the sequence includes an alpha helical structure, which may or may not include a cytotoxic moiety or domain such as in a fusion construct. In various aspects, a nucleolin-binding peptide or fusion construct contains a continuous alpha helical structure that spans at least 30% of the length of the peptide or fusion construct. In more particular aspects, a nucleolin-binding peptide or fusion construct contains a alpha helical structure that spans 30-40%, 40-50%, 50-60%, 60-70%, 70-80%, 80-90%, or 90-100%, continuous residues in length of the peptide, which peptides may or may not include a cytotoxic moiety or domain such as in a fusion construct.

Peptides and fusion constructs of the invention also include sequences in which all or a part of the sequence is amphipathic. Peptides and fusion constructs of the invention further include sequences in which all or a part of the sequence is cationic. Such sequences may also form an alpha helical structure, at least over a portion of the full length sequence, which may or may not include a cytotoxic moiety or domain such as in a fusion construct.

As disclosed herein, nucleolin binding peptides can be linked or conjugated to one or more additional entities, domains or moieties. In one embodiment, a nucleolin binding peptide is linked or conjugated (fused) to a cytotoxic moiety or domain, such as a lytic peptide. In another embodiment, a nucleolin binding peptide is linked or conjugated (fused) to a detectable agent or a tag. Such “fusion constructs” can be used to target nucleolin expressing cells for lysis or death, or for detection or diagnosis of, or screening for, the presence of nucleolin expressing cells.

Exemplary cytotoxic moieties or domains joined to a nucleolin binding peptide include or consist of an amino acid sequence, chemotherapeutic drug, a radionuclide, plant or bacterial toxin, or plant or bacterial toxin fragment, a pore-forming peptide, or a toxin. Cytotoxic (lytic) peptides are typically positively charged, amphipathic sequences containing amino acids of various lengths, and encompass a broad array of different peptide sequences. Exemplary cytotoxic (lytic) domains joined to a nucleolin binding peptide include or consists of an amino acid sequence selected from KFAKFAKKFAKFAK (Phor14), KFAKFAKKFAKFAKK, KFAKFAKKFAKFAKKF, KFAKFAKKFAKFAKKFA, KFAKFAKKFAKFAKKFAK, KFAKFAKKFAKFAKKFAKF, KFAKFAKKFAKFAKKFAKFA and KFAKFAKKFAKFAKKFAKFAK (Phor21); or an amino acid sequence selected from KFAKFAKKFAKFAK (Phor14), KFAKFAKKFAKFAKK, KFAKFAKKFAKFAKKF, KFAKFAKKFAKFAKKFA, KFAKFAKKFAKFAKKFAK, KFAKFAKKFAKFAKKFAKF, KFAKFAKKFAKFAKKFAKFA and KFAKFAKKFAKFAKKFAKFAK (Phor21) having one or more of the K residues substituted with any of an F or L residue, one or more of the F residues substituted with any of a K, A or L residue, or one or more of the A residues substituted with any of a K, F or L residue; or include or consist of a 12 to 28 amino acid sequence that includes a peptide selected from KFAKFAKKFAKFAK (Phor14), KFAKFAKKFAKFAKK, KFAKFAKKFAKFAKKF, KFAKFAKKFAKFAKKFA, KFAKFAKKFAKFAKKFAK, KFAKFAKKFAKFAKKFAKF, KFAKFAKKFAKFAKKFAKFA and KFAKFAKKFAKFAKKFAKFAK (Phor21), or a 12 to 28 amino acid sequence that includes a peptide selected from KFAKFAKKFAKFAK (Phor14), KFAKFAKKFAKFAKK, KFAKFAKKFAKFAKKF, KFAKFAKKFAKFAKKFA, KFAKFAKKFAKFAKKFAK, KFAKFAKKFAKFAKKFAKF, KFAKFAKKFAKFAKKFAKFA and KFAKFAKKFAKFAKKFAKFAK (Phor21) having one or more of the K residues substituted with any of an F or L residue, one or more of the F residues substituted with any of a K, A or L residue, or one or more of the A residues substituted with any of a K, F or L residue.

Additional exemplary cytotoxic (lytic) domains that can be joined to a nucleolin binding peptide include or consist of an amino acid sequence of anti-microbial defense peptides and analogs and hybrids (chimeras) of such peptides. Such peptides include all or a portion of a sequence of a cecropin, defensin, melittin, sarcotoxin, or magainin peptide, and analogs and hybrids (chimeras) of such peptides.

An exemplary non-limiting example of cytotoxic (lytic) domains that can be joined to a nucleolin binding peptide include or consist of hecate, FALALKALKKALKKLKKALKKAL (hecate), or a subsequence thereof, which is an amphipathic lytic peptide that acts on cell membranes without being internalized, and is a synthetic peptide analog of melittin, the primary toxin in honeybee venom. Additional non-limiting examples of cytotoxic (lytic) domains that can be joined to a nucleolin binding peptide include or consist of Phe Ala Phe Ala Phe Lys Ala Phe Lys Lys Ala Phe Lys Lys Phe LysLys Ala Phe Lys Lys Ala Phe (D1A21); Phe Ala Lys Lys Phe Ala Lys Lys Phe Lys Lys Phe Ala Lys Lys Phe Ala Lys Phe Ala Phe Ala Phe (D2A21): Lys Arg Lys Arg Ala Val Lys Arg Val Gly Arg Arg Leu Lys Lys Leu Ala Arg Lys Ile Ala Arg Leu Gly Val Ala Phe (D5C); and Lys Arg Lys Arg Ala Val Lys Arg Val Gly Arg Arg Leu Lys Lys Lee Ala Arg Lys Be Ala Arg Leu Gly Val Ala Lys Leu Ala Gly Len Arg Ala Val Leu Lys Phe (D5C1), or subsequences thereof.

Further non-limiting examples of cytotoxic (lytic) domains that can be joined to a nucleolin binding peptide include or consist of lytic peptides as described (e.g., Boman et al., Curr. Top. Microbial. Immunol., 94/95:75-91 (1981); Boman et al., Annu. Rev. Microbiol., 41:103 (1987); Zasloff, Proc. Natl. Acad. Sci. USA, 84:3628 (1987); Ganz et al., J. Chin. Invest., 76:1427 (1985); and Lee et al., Proc. Natl. Acad. Sci. USA, 86:9159 (1989)). Lytic peptides and their sequences are also described in Yamada et al., Biochem. J., 272:633 (1990); Taniai et al., Biochimica Et Biophysica Acta, 1132: 203 (1992); Boman et al., Febs Lett., 259:103 (1989); Tessier et al., Gene, 98:177 (1991); Blondelle et al., Biochemistry, 30: 4671 (1991); Andreu et al., Febs Lett., 296:190 (1992); Macias et al., Can. J. Microbiol., 36:582 (1990); Rana et al., Biochemistry, 30:5858 (1991); Diamond et al., Proc. Natl. Acad. Sci. USA, 90:4596 ff (1993); Selsted et al., J. Biol. Chem., 268:6641 ff (1993); Tang et al., J. Biol. Chem., 268:6649 ff (1993); Lehrer et al., Blood 76:2169 (1990); Ganz et al., Sem, Resp. Infect. I, pp. 107-117 (1986); Kagan et al., Proc. Natl. Acad. Sci. USA, 877:210 (1990); Wade et al., Proc. Natl. Acad. Sci. USA, 87:4761 (1990); Romeo et al., J. Biol. Chem., 263:9573 (1988); Jaynes et al., WO 89/00199 (1989); Jaynes, WO 90/12866 (1990); and Berkowitz, WO 93/01723 (1993).

Cecropins, such as cecropin A, cecropin B, and cecropin D, are typically small, highly homologous, basic peptides. The amino-terminal half of various cecropins contains a sequence that will form an amphipathic alpha-helix, and the carboxy-terminal half of the peptide comprises a hydrophobic tail. A cecropin-like peptide has been isolated from porcine intestine (Lee et al., Proc. Natl. Acad. Sci. USA, 86:9159 (1989)). Cecropin peptides have been reported to kill a number of animal pathogens other than bacteria (Jaynes et al., FASEB J., 2878-2883 (1988); Arrowood et al., J. Protozool., 38, No. 6, 161S-163S (1991); and Arrowood et al., Antimicrob. Agents Chemother., 35: 224 (1991)). Normal mammalian cells do not appear to be adversely affected by cecropins, even at high concentrations (Jaynes et al., Peptide Research, 2, No. 2, pp. 1-5 (1989); and Reed et al., Mol. Reprod. Devel., 31, No. 2, pp. 106-113 (1992)). The synthetic lytic peptide known as S-1 (or Shiva 1) has been reported to destroy intracellular Brucella abortus-, Trypanosoma cruzi-, Cryptosporidium parvum-, and infectious bovine herpes virus I (IBR)-infected host cells, with little or no toxic effects on noninfected mammalian cells (Jaynes et al., Peptide Research, 2, No. 2, pp. 1-5 (1989); Wood et al., Proc. Ann. Amer. Soc. Anim. Sci., Utah State University, Logan, Utah J. Anim. Sci. (Suppl. 1), vol. 65, p. 380 (1987); Arrowood et al., J. Protozoa, 38, No. 6, pp. 161S-163S (1991); Arrowood et al., Antimicrob. Agents Chemother., 35:224 (1991); and Reed et al., Mol. Reprod. Devel., 31, No. 2, pp. 106-113 (1992)).

Defensins, originally found in mammals, are typically small peptides containing six to eight cysteine residues (Ganz et al., J. Clin. Invest., 76:1427 (1985)). Extracts of normal human neutrophils contain three defensin peptides: human neutrophil peptides HNP-1, HNP-2, and HNP-3. Defensin peptides have also been reported in insects and higher plants (Dimarcq et al., EMBO J., 9:2507 (1990); and Fisher et al., Proc. Natl. Acad. Sci. USA, 84:3628 (1987).

Sarcotoxins are typically slightly larger peptides, and have been purified from the fleshfly Sarcophaga peregrine (Okada et al., J. Biol. Chem., 260:7174 (1985). Although highly divergent from the cecropins and defensins, sarcotoxins appear to have a similar antibiotic function.

Other lytic peptides have been reported in amphibians. Two peptides from the African clawed frog, Xenopus laevis, named PGS and Gly¹⁰Lys²²PGS (Gibson et al., J. Biol. Chem., 261:5341 (1986); and Givannini et al., Biochem. J., 243:113 (1987)). Xenopus-derived peptides have been reported to have antimicrobial activity, which had been renamed magainins (Zasloff, Proc. Natl. Acad. Sci. USA, 84:3628 (1987)). Other reports the in vitro use of a magainin to selectively reduce the viability of the parasite Bonamia ostreae at doses that did not affect cells of the flat oyster Ostrea edulis (Morvan et al., Mol. Mar. Biol., 3:327 (1994)).

Additional cytotoxic moieties or domains joined to a nucleolin binding peptide include or consist of synthetic peptides disclosed in U.S. Pat. Nos. 6,656,334; 6, 635,740 and 5,789,542.

A nucleolin binding peptide is linked or conjugated to one or more additional entities, moieties or domains indirectly or directly, by a covalent or by a non-covalent bond. An indirect linkage includes an intermediary, such as a linker, or a peptide or non-peptide joins one or more additional entities, moieties or domains to a nucleolin binding peptide in a fusion. In particular aspects, a linker is a peptide sequence having from about 1 to 25 amino acid residues, or having a linear carbon chain. In additional particular aspects, a peptide is joined by a peptide sequence that includes or consist of one or more A, S or G amino acid residues. In further particular aspects, a peptide is joined by a sequence including or consisting of GSGGS, ASAAS, or an aliphatic carbon chain such as CX (where X is the number of carbons), e.g., C6, or CCCCCC. Other peptide linkers include but are not limited to GS, AF, FK, VK, FFK, FA, GSGRSA, RVRRSV, SS, Cit-V, F-Cit, at various length. Thioether, N-succinidyl-3-(2-pyridylothio)propionate, thio ether bonds such as SIAB [N-succinimidyl (4-iodoacetyl) aminobenzoate], SMCC [succinimidyl-4-(N-maleimidomethyl)cyclohexane-1-carboxylate], MBS (m-Maleimidobenzoyl-N-hydroxysuccinimide) and SMPB [succinimidyl-4-(p-maleimidophenyl)butyrate], maleimide and hydrazone linkers.

Peptides of the invention, and a nucleolin binding peptide or cytotoxic domain (e.g., lytic domain sequence), all or in part, can include or consist of an amino acid, or an amino acid sequence. In particular aspects, a peptide of the invention, or a nucleolin binding peptide or cytotoxic moiety or domain (e.g., lytic domain sequence) has about 5 to 10 (i.e., 5, 6, 7, 8, 9, 10 amino acids), 10 to 20 (i.e., 10, 11, 12, 13, 14, 15 16, 17, 18, 19 or 20 amino acids), 15 to 20 (i.e., 15, 16, 17, 18, 19 or 20 amino acids), 20 to 30, 30 to 40, 40 to 50, 60 to 70, 70 to 80, 80 to 90, 90 to 100 or more amino acid residues.

In a fusion construct, a nucleolin binding peptide or cytotoxic moiety or domain can be positioned at either the N-terminus or the C-terminus relative to the other or any other second, third, fourth, fifth or subsequent domain. In one embodiment, a nucleolin binding peptide is positioned at the N-terminus relative to the cytotoxic moiety or domain, and in another embodiment, a nucleolin binding peptide is positioned at the C-terminus relative to the cytotoxic moiety or domain.

Nucleolin binding peptides or cytotoxic moieties or domains can include or consist of one or more L- or D-amino acids. In particular aspects, a nucleolin binding peptide or cytotoxic domain has an L- or D-amino acid at any one, 1-5, 5-10, 10 to 20 (i.e., 10, 11, 12, 13, 14, 15 16, 17, 18, 19 or 20 amino acids), 15 to 20 (i.e., 15, 16, 17, 18, 19 or 20 amino acids), 20 to 30, 30 to 40, 40 to 50, 60 to 70, 70 to 80, 80 to 90, 90 to 100 or more amino acid residues.

Nucleolin binding peptides and fusion constructs include or consist of isolated and purified forms. Nucleolin binding peptides and fusion constructs also include or consist of a mixture. Such mixtures include compositions, such as a mixture or formulation of a nucleolin binding peptide or fusion construct, such as a pharmaceutically acceptable carrier or excipient appropriate for administration to or in vivo contact with a subject, or a mixture of a nucleolin binding peptide or fusion construct and an anti-cell proliferative, immune stimulating or anti-angiogenic or anti-inflammatory agent.

Nucleolin binding peptides and fusion constructs can be included in a unit dosage form. In one embodiment, a nucleolin binding peptide or fusion construct is in a unit dosage in an amount effective to treat a subject having undesirable cell proliferation or a hyperproliferative disorder or undesirable or aberrant angiogenesis or inflammation. In another embodiment, a nucleolin binding peptide or fusion construct is in a unit dosage in an amount effective to treat a subject having a neoplasia, tumor or cancer. In an additional embodiment, a nucleolin binding peptide or fusion construct is in a unit dosage in an amount effective to reduce, inhibit or decrease angiogenesis, an angiogenesis related or dependent disease, or an inflammatory disease.

Nucleolin binding peptides and fusion constructs can be included within kits, optionally with instructions for practicing a method. In one embodiment, a kit includes a nucleolin binding peptide or fusion construct and instructions for reducing or inhibiting proliferation of a cell, reducing or inhibiting proliferation of a hyperproliferating cell, reducing or inhibiting proliferation of a neoplastic, tumor or cancer cell, treating a subject having a hyperproliferative disorder, treating a subject having a neoplasia, tumor or cancer, or reducing, inhibiting or decreasing angiogenesis, an angiogenesis related or dependent disease, or an inflammatory disease or inflammation.

In accordance with the invention, there are also provided nucleic acids that encode nucleolin binding peptides and fusion constructs. Nucleic acids can be included in a vector, such as an expression vector that when expressed in a cell encodes a nucleolin binding peptide or fusion construct. Host cells can be transformed with a nucleic acid in a vector, such that the cell expresses a nucleolin binding peptide or fusion construct encoded by the nucleic acid.

Nucleolin binding peptides and fusion constructs are useful for, among other things, reducing or inhibiting proliferation of a nucleolin expressing cell, reducing or inhibiting proliferation of a hyperproliferating nucleolin expressing cell, reducing or inhibiting proliferation of a neoplastic, tumor, cancer or malignant cell, reducing or inhibiting proliferation of endothelial cells, and treating undesirable or aberrant cell proliferation, such as hyperproliferating cells, hyperproliferative disorders and angiogenesis related or dependent diseases and disorders, or an inflammatory disease or disorder. Non-limiting examples of hyperproliferative disorders include benign hyperplasia, non-metastatic and metastatic neoplasia, cancers tumors and malignancies (e.g., a solid or liquid tumor, myeloma, lymphoma, leukemia, carcinoma, sarcoma, melanoma, neural, reticuloendothelial and haematopoietic malignancies).

In accordance with the invention, there are provided methods of reducing or inhibiting proliferation of a nucleolin expressing cell; methods of reducing or inhibiting nucleolin expressing cell proliferation; methods of reducing or inhibiting proliferation of a nucleolin expressing hyperproliferating cell; methods of reducing or inhibiting proliferation of a neoplastic, tumor, cancer or malignant cell; and methods of reducing or inhibiting proliferation of endothelial cells. In various embodiments, a method includes contacting a cell with a nucleolin binding peptide or fusion construct in an amount sufficient to reduce or inhibit proliferation of the cell; contacting a cell with a nucleolin binding peptide or fusion construct in an amount sufficient to reduce or inhibit cell proliferation; contacting a cell with a nucleolin binding peptide or fusion construct in an amount sufficient to reduce or inhibit proliferation of the hyperproliferating cell; contacting a cell with a nucleolin binding peptide or fusion construct in an amount sufficient to reduce or inhibit proliferation of the neoplastic, tumor, cancer or malignant cell; and contacting a cell with a nucleolin binding peptide or fusion construct in an amount sufficient to reduce or inhibit proliferation of endothelial cells.

In accordance with the invention, there are moreover provided methods of selectively reducing or inhibiting proliferation of a cell that expresses nucleolin; selectively reducing or inhibiting proliferation of a hyperproliferating cell that expresses nucleolin; selectively reducing or inhibiting proliferation of a neoplastic, tumor, cancer or malignant cell that expresses nucleolin; and selectively reducing or inhibiting proliferation of cells that are associated with or participate in angiogenesis, such as endothelial cells. In various embodiments, a method includes contacting a cell with a nucleolin binding peptide or fusion construct in an amount sufficient to reduce or inhibit proliferation of the cell; contacting a cell with a nucleolin binding peptide or fusion construct in an amount sufficient to reduce or inhibit proliferation of the hyperproliferating cell; contacting a cell with a nucleolin binding peptide or fusion construct in an amount sufficient to reduce or inhibit proliferation of the neoplastic, tumor, cancer or malignant cell; and contacting a cell associated with or that participates in angiogenesis in an amount sufficient to reduce or inhibit proliferation of the cell.

Cells targeted in accordance with the invention methods include cells that express nucleolin. Non-limiting examples of nucleolin expressing cells include hyperproliferating cells; neoplastic, tumor, cancer and malignant cells; and cells associated with or that participate in angiogenesis, such as endothelial cells, as such cells comprise vasculature that provides blood supply to hyperproliferating cells and by targeting endothelial cells for cytotoxicity can in turn result in cell death and killing of hyperproliferating cells to which the vasculature provides blood even if the hyperprolferating cells do not express nucleolin.

Methods performed include, among others, contacting a subject in need of inhibiting, reducing or preventing proliferation, survival, differentiation, death, or activity of nucleolin expressing cells, such as a hyperprolifertive cell or an undesirably proliferating cell. Exemplary subjects include a subject having or at risk of having undesirable or aberrant cell proliferation; a subject having or at risk of having a benign hyperplasia; or a non-metastatic or metastatic neoplasia, cancer, tumor or malignancy (e.g., a solid or liquid tumor, myeloma, lymphoma, leukemia, carcinoma, sarcoma, melanoma, neural, reticuloendothelial and haematopoietic neoplasia); and a subject having or at risk of having an angiogenesis related or dependent disease or disorder, or an inflammatory disease or disorder.

In accordance with the invention, there are provided methods of treating a subject having a hyperproliferative disorder, methods of treating a subject having a neoplasia, tumor, cancer or malignancy (metastatic, non-metastatic or benign), and methods of treating a subject having an angiogenesis related or dependent disease or disorder, or an inflammatory disease or disorder. In various embodiments, a method includes, administering to a subject an amount of nucleolin binding peptide or fusion construct sufficient to treat the hyperproliferative disorder; and administering to a subject an amount of nucleolin binding peptide or fusion construct sufficient to reduce or inhibit proliferation of the hyperproliferating cells, the neoplasia, tumor, cancer or malignancy, or administering to a subject an amount of nucleolin binding peptide or fusion construct sufficient to reduce or inhibit proliferation of the cells comprising the angiogenesis related or dependent disease or disorder, or inflammatory disease or disorder.

Methods include treating a subject having or at risk of having a metastasis. For example, an amount of a nucleolin binding peptide or fusion construct effective to reduce or inhibit spread or dissemination of a tumor, cancer or neoplasia to other sites, locations or regions within the subject. In various embodiments, a method reduces or inhibits metastasis of a primary tumor or cancer to one or more other sites, formation or establishment of a metastasis at one or more other sites, locations or regions thereby reducing or inhibiting tumor or cancer relapse or tumor or cancer progression. In further embodiments, a method reduces or inhibits growth, proliferation, mobility or invasiveness of tumor or cancer cells that potentially or do develop metastases (e.g., disseminated tumor cells); reduces or inhibits formation or establishment of metastases arising from a primary tumor or cancer to one or more other sites, locations or regions distinct from the primary tumor or cancer; reduces or inhibits growth or proliferation of a metastasis at one or more other sites, locations or regions distinct from the primary tumor or cancer after the metastasis has formed or has been established; or reduces or inhibits formation or establishment of additional metastasis after the metastasis has been formed or established. In yet another embodiment, a method reduces or inhibits relapse or progression of the neoplasia, tumor, cancer or malignancy.

In accordance with the invention, there are still further provided methods of reducing or inhibiting metastasis of a neoplasia, tumor, cancer or malignancy to other sites, or formation or establishment of metastatic neoplasia, tumor, cancer or malignancy at other sites distal from a primary neoplasia, tumor, cancer or malignancy. In various embodiments, a method includes administering to a subject an amount of a nucleolin binding peptide or fusion construct sufficient to reduce or inhibit metastasis of the neoplasia, tumor, cancer or malignancy to other sites, or formation or establishment of metastatic neoplasia, tumor, cancer or malignancy at other sites distal from the primary neoplasia, tumor, cancer or malignancy.

Hyperproliferating cells, neoplasia, tumor, cancer, malignancy, an angiogenesis related or dependent disease or disorder, or an inflammatory disease or disorder treatable in accordance with the invention include solid cellular mass, hematopoietic cells, or a carcinoma, sarcoma (e.g. lymphosarcoma, liposarcoma, osteosarcoma, chondrosarcoma, leiomyosarcoma, rhabdomyosarcoma or fibrosarcoma), lymphoma, leukemia, adenoma, adenocarcinoma, melanoma, glioma, glioblastoma, meningioma, neuroblastoma, retinoblastoma, astrocytoma, oligodendrocytoma, mesothelioma, reticuloendothelial, lymphatic or haematopoietic (e.g., myeloma, lymphoma or leukemia) neoplasia, tumor, cancer or malignancy.

Hyperprolferating cells, neoplasia, tumor, cancer, malignancy, an angiogenesis related or dependent disease or disorder, or an inflammatory disease or disorder treatable in accordance with the invention can be present in or affect a lung (small cell lung or non-small cell lung cancer), thyroid, head or neck, nasopharynx, throat, nose or sinuses, brain, spine, breast, adrenal gland, pituitary gland, thyroid, lymph, gastrointestinal (mouth, esophagus, stomach, duodenum, ileum, jejunum (small intestine), colon, rectum), genito-urinary tract (uterus, ovary, cervix, endometrial, bladder, testicle, penis, prostate), kidney, pancreas, liver, bone, bone marrow, lymph, blood, muscle, skin or stem cell neoplasia, tumor, cancer, or malignancy.

Methods may be practiced with other treatments or therapies (e.g. surgical resection, radiotherapy, ionizing or chemical radiation therapy, chemotherapy, immunotherapy, local or regional thermal (hyperthermia) therapy, or vaccination). Such treatments or therapies can be administered prior to, substantially contemporaneously with (separately or in a mixture), or following administration of a nucleolin binding peptide or fusion construct. In one embodiment, a method includes administering an anti-cell proliferative, anti-neoplastic, anti-tumor, anti-cancer or immune-enhancing treatment or therapy, an anti-angiogenic treatment or therapy, or an inflammatory treatment or therapy. In further embodiments, a method includes administering an alkylating agent, anti-metabolite, plant extract, plant alkaloid, nitrosourea, hormone, nucleoside or nucleotide analog; cyclophosphamide, azathioprine, cyclosporin A, prednisolone, melphalan, chlorambucil, mechlorethamine, busulphan, methotrexate, 6-mercaptopurine, thioguanine, 5-fluorouracil, cytosine arabinoside, 5-azacytidine (5-AZC) and 5-azacytidine related compounds, bleomycin, actinomycin D, mithramycin, mitomycin C, carmustine, lomustine, semustine, streptozotocin, hydroxyurea, cisplatin, carboplatin, oxiplatin, mitotane, procarbazine, dacarbazine, taxol, vinblastine, vincristine, doxorubicin or dibromomannitol, topoisomerase inhibitors, (irinotecan, topotecan, etoposide, teniposide), gemcitabine, pemetrexed etc. Cell or immunotherapies include lymphocytes, plasma cells, macrophages, dendritic cells, T-cells, NK cells or B-cells; antibodies, vaccines, a cell growth factor, a cell survival factor, a cell differentiative factor, a cytokine or a chemokine(examples are interleukins IL-2, IL-1α, IL-1β, IL-3, IL-6, IL-7, granulocyte-macrophage-colony stimulating factor (GMCSF), IFN-γ, IL-12, TNF-α, TNFβ, MIP-1α, MIP-1β, RANTES, SDF-1, MCP-1, MCP-2, MCP-3, MCP-4, eotaxin, eotaxin-2, I-309/TCA3, ATAC, HCC-1, HCC-2, HCC-3, LARC/MIP-3α, PARC, TARC, CKβ, CKβ6, CKβ7, CKβ8, CKβ9, CKβ11, CKβ12, C10, IL-8, GROα, GROβ, ENA-78, GCP-2, PBP/CTAPIIIβ-TG/NAP-2, Mig, PBSF/SDF-1, or lymphotactin) etc.

Additional agents that are applicable with a composition that includes, or contact or administration of a nucleolin binding peptide or fusion construct, are targeted drugs or biologicals such as antibodies or small molecules. Non-limiting examples of monoclonal antibodies include rituximab (Rituxan®), trastuzumab (Herceptin®), bevacizumab (Avastin®), cetuximab (Erbitux®), alemtuzumab (Campath®), panitumumab (Vectibix®), ibritumomab tiuxetan (Zevalin®), tositumomab (Bexxar®) etc. which can be used in combination with, inter alia, a nucleolin binding peptide or fusion construct in accordance with the invention. Other targeted drugs that are applicable for use with a nucleolin binding peptide or fusion construct are imatinib (Gleevec®), gefitinib (Iressa®), bortzomib (Velcade®), lapatinib (Tykerb®), sunitinib (Sutent®), sorafenib (Nexavar®), nilotinib (Tasigna®), Erlotinib hydrochloride (Tarceva®) etc.

Methods of the invention include providing a subject with a benefit. In particular embodiments, a method of treatment results in partial or complete destruction of nucleolin expressing cells, such as hyperproliferating, neoplastic, tumor, cancer or malignant, or angiogenic (endothelial) cell mass, volume, size or numbers of cells; stimulating, inducing or increasing cell necrosis, lysis or apoptosis, reducing cell volume or size, cell mass; inhibiting or preventing progression or an increase in cell volume, mass, size or cell numbers, or prolonging lifespan; reducing or decreasing severity, duration or frequency of an adverse symptom or complication associated with or caused by nucleolin expressing cells; reducing or decreasing pain, discomfort, nausea, weakness or lethargy; or increased energy, appetite, improved mobility or psychological well being.

In accordance with the invention, there are still additionally provided methods of reducing or inhibiting angiogenesis. In various embodiments, a method includes contacting a cell (e.g., an endothelial cell) with an amount of nucleolin binding peptide or fusion construct sufficient to reduce or inhibit angiogenesis.

In accordance with the invention, there are still further provided methods of reducing or inhibiting migration of endothelial cells or capillary-tubule formation. In various embodiments, a method includes contacting endothelial cells with a nucleolin binding peptide or fusion construct sufficient in an amount sufficient to reduce or inhibit migration of endothelial cells or capillary-tubule formation.

In accordance with the invention, there are still moreover provided methods of treating a subject having an angiogenesis related or dependent disease. In various embodiments, a method includes administering to the subject an amount of a nucleolin binding peptide or fusion construct sufficient to reduce or inhibit angiogenesis thereby treating the angiogenesis related or dependent disease. An angiogenesis related or dependent disease treatable in accordance with the invention includes, for example, a vascular tumor, cancer, neoplasia or malformation; a reproductive disorder; a cardiovascular, pulmonary or central nervous system disease; a syndrome; an ocular disorder; a chronic inflammatory disease, aberrant wound repair or a metabolic disorder. Non-limiting specific examples of angiogenesis related or dependent diseases include a solid or liquid tumor, cancer, neoplasia or malformation; angiofibroma, arteriovenous malformation, hemangiomatosis; Endometriosis, Placental insufficiency, Pre-eclampsia, Fibroid, Atherosclerosis, Vascular adhesions, Vascular dementia, Restenosis/reperfusion injury, Pulmonary fibrosis, Alzheimer's disease, CADASIL (cerebral autosomally dominant arteriopathy with subcortical infarcts and leucoencephalopathy), Dyschondroplasia with vascular hematomas (Maffucci's Syndrome), Hereditary hemorrhagic telangiectasia (Rendu-Osler-Weber Syndrome), Von Hippel-Lindau Syndrome, Corneal graft neovascularization, Diabetic retinopathy, Ischemic retinopathy, Neovascular glaucoma, Retrolental fibroplasia, Retinopathy of prematurity, Trachoma, Macular degeneration, Inflammatory Bowel Disease, Crohn's disease, Ulcerative Colitis, Diabetes types I or II, Granulations-burns, Hemophiliac joints, Hypertrophic scars, Nonunion fractures, Obesity, Osteoradionecrosis, Psoriasis, Pyogenic granuloma, Periodontitis, Rheumatoid arthritis, Systemic sclerosis, etc.

Methods include those performed in vitro (with a sample or with cells), ex vivo and in vivo (i.e., in a subject). Subjects treatable in accordance with the methods include mammals, such as a human.

### BRIEF DESCRIPTION OF DRAWINGS

FIG. 1 shows the sequence of 31 amino acid F-3 fragment, the 14 amino acid F3 fragment and the new helical ligand peptide 302L. The arrow shows the helical span of the designed molecule. The 302L ligand is an alpha helical structure absent in the F3 sequence. The arrow shows the helical region, K and R are positively charged residues, and D and E are negatively charged residues.

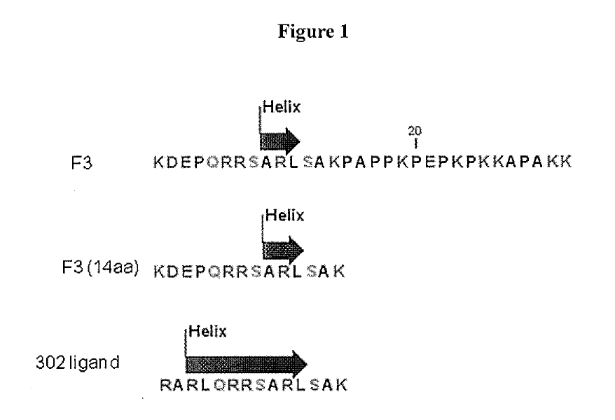

FIGS. 2A-2B show a sequence analysis of (A) EP-301 and (B) EP-302. EP-301 has a discontinuous alpha helical structure (bold arrows) from amino acids 19-22 (KDEP). EP-302 has RARL substituted for KDEP to produce a continuous helical structure (bold arrow).

FIGS. 3A-3B show a graph depicting: (A) Kinetics of cell killing of EP-301 and EP-302 incubated cells at concentrations of 0.0001, 0.001, 0.01, 0.1, 1, 5, 10, 50 and 100 μM; and (B) Hemolytic activity of of EP-301 and EP-302 towards human red blood cells incubated for 2 hours.

FIG. 4 is a bar graph showing competition of EP-302L with EP-302 for binding to the nucleolin-target. Increasing concentrations of EP-302L reduced cytotoxicity of EP-302 to MDA-MB-435S cells (passage #249). MDA-MB-435S cells express nucleolin on the cell surface, which is blocked by pre-incubation (1 h) with EP-302L prior to addition of EP-302 to cells. Total incubation EP-302 6 h. p<0.001 vs EP-302L alone.

FIGS. 5A-5B show a graph of data indicating that EP-302 caused tumor volume reduction in (A) PC-3 xenografts and in (B) MDA-MB-435S xenograft models. N=10 for MDA-MB-435, N=16 for PC-3. Treatments: Day 17, 21, 25 and 30.

FIGS. 6A-6B show cytotoxicity of Phor18 anti-nucleolin antibody conjugates to MDA-MB-435S, MiaPaCa, 3T3, HL60, U937 and Jurkat cells (nanomolar IC₅₀) (ADC) after 15 h.

### DETAILED DESCRIPTION

The invention provides peptides that bind to nucleolin, peptides that bind to nucleolin expressed by cells and peptides that bind to nucleolin and that are optionally cytotoxic (i.e., “lytic”) towards cells expressing nucleolin. The invention peptides that are cytotoxic towards cells have cytotoxic activity alone. The peptides that bind nucleolin exhibit cytotoxicity to the cells without addition or linkage of a cytotoxic moiety or domain (e.g., lytic peptide). Such peptides of the invention have cell cytotoxicity alone, and need not include or be linked to a cytotoxic moiety or domain (e.g., lytic domain sequence). Such peptides bind to cells expressing nucleolin and can target such cells for lysis or death. The invention peptides that bind to nucleolin, but do not have detectable cell cytotoxicity alone, can be made cytotoxic towards nucleolin expressing cells with the addition or linkage of a cytotoxic moiety or domain (e.g., lytic peptide), such as in a fusion construct. Such peptides bind to nucleolin but may or may not have cytotoxic activity without a separate or additional cytotoxic moiety or domain (e.g., lytic domain sequence) that stimulates, promotes or induces cell lysis or death. Peptides that bind to nucleolin having or not having cell cytotoxicity can be linked to a second separate or additional cytotoxic moiety or domain or region (e.g., lytic domain sequence) that is cytotoxic towards cells, and stimulates, promotes or induces cell lysis or death.

Peptides of the invention include sequences with varying sequence identity to a part of high mobility group nucleosomal binding domain 2 (HMGN2) sequence, which binds to nucleolin. For example, a 31 amino acid peptide sequence from the N-terminal of high mobility group nucleosomal binding domain 2 (HMGN2) has been reported to bind to nucleolin. A portion of the 31 amino acid sequence, and a modified peptide sequence that is less than 100% identical to a portion of the 31 amino acid peptide sequence, retains binding to nucleolin. The modified sequence and variants thereof disclosed herein bind to nucleolin as well as exhibit cytotoxicity towards nucleolin expressing cells. In addition, a modified peptide sequence that bears some identity to a portion of the 31 amino acid peptide sequence retains binding to nucleolin, when combined with a second separate or additional cytotoxic moiety or domain or region (a lytic domain sequence), was also cytotoxic towards nucleolin expressing cells.

Although not wishing to be bound by any theory, peptides of the invention that bind nucleolin and that are cytotoxic towards cells that express nucleolin are believed to disrupt the cell membrane, which results in cell lysis or death. Peptide cytotoxicity appears to depend in part on the presence of an alpha helical structure that spans at least a continuous portion of the peptide sequence. Since the alpha helix makes a complete turn for every 3.6 residues, the amino acid sequence of an amphipathic alpha helix alternates between hydrophilic and hydrophobic residues every 3 to 4 residues. A continuous alpha helical structure, that spans at least a portion of the peptide sequence, may therefore provide a structure that has cell cytotoxicity. An amphipathic alpha-helix contains mostly hydrophilic amino acids on one side of the alpha-helix and the other side contains mostly hydrophobic amino acids. A PNNPNNP repeat pattern or motif is predicted to form an amphipathic alpha-helix where P represents a positively charged amino acid residue and N a neutral amino acid residue. A PNNPNNP repeat pattern provides a cationic binding site for the lytic peptide to a negatively charged cell membrane and a hydrophobic site for membrane interaction/penetration. Such peptides that therefore have at least a portion of their sequence (e.g., 30% or more) with a continuous stretch of amino acids that form a helical structure are cytotoxic and can stimulate, increase, promote or induce cell lysis, death, killing or apoptosis, or cell cycle arrest.

In accordance with the invention, there are provided nucleolin binding peptides and fusion constructs. In one embodiment, a peptide or a fusion construct that binds to nucleolin includes a continuous amino acid sequence with the general formula, X1-X2-X3-X4-X5-X6-X7-X8-X9-X10-X11-X12-X13-X14, wherein: X1 is R, H, G, K or V; X2 is A, G or V; X3 is R, K or H; X4 is L; X5 is Q; X6 is R; X7 is R; X8 is S, F or L; X9 is A; X10 is R; X11 is L or nothing (absent); X12 is S, For L or nothing (absent); X13 is A or nothing (absent); X14 is K or nothing (absent). In more particular embodiments, a peptide or a fusion construct that binds to nucleolin includes a sequence: RARLQRRSARLSAK, KARLQRRSARLSAK, VARLQRRSARLSAK, HARLQRRSARLSAK, GARLQRRSARLSAK, RAKLQRRSARLSAK, HAHLQRRSARLSAK, RARLQRRFARLFAK, KARLQRRFARLFAK, VARLQRRFARLFAK, GARLQRRFARLFAK, HARLQRRFARLFAK, HAHLQRRFARLFAK, RAKLQRRFARLFAK, RARLQRRLARLLAK, KARLQRRLARLLAK, VARLQRRLARLLAK, GARLQRRLARLLAK, HARLQRRLARLLAK, RAKLQRRLARLLAK, HAHLQRRLARLLAK, RARLQRRSARLSA, KARLQRRSARLSA, VARLQRRSARLSA, HARLQRRSARLSA, GARLQRRSARLSA, RAKLQRRSARLSA, HAHLQRRSARLSA, RARLQRRFARLFA, KARLQRRFARLFA, VARLQRRFARLFA, GARLQRRFARLFA, HARLQRRFARLFA, HAHLQRRFARLFA, RAKLQRRFARLFA, RARLQRRLARLLA, KARLQRRLARLLA, VARLQRRLARLLA, GARLQRRLARLLA, HARLQRRLARLLA, RAKLQRRLARLLA, HAHLQRRLARLLA, RARLQRRSAR, KARLQRRSAR, VARLQRRSAR, HARLQRRSARL, GARLQRRSAR, RAKLQRRSAR, HAHLQRRSAR, RARLQRRSAR, KARLQRRFAR, VARLQRRFAR, GARLQRRFARLFA, HARLQRRFAR, HAHLQRRFAR, RAKLQRRFAR, RARLQRRLAR, KARLQRRLAR, VARLQRRLAR, GARLQRRLAR, HARLQRRLAR, RAKLQRRLAR, HAHLQRRLAR, RGRLQRRSARLSAK, RVRLQRRSARLSAK, RGRLQRRSARLSA, RVRLQRRSARLSA, RGRLQRRSARLS, RVRLQRRSARLS, RGRLQRRSARL, RVRLQRRSARL, RGRLQRRSAR, or RVRLQRRSAR; or a sequence: RARLQRRSARLSAK, KARLQRRSARLSAK, VARLQRRSARLSAK, HARLQRRSARLSAK, GARLQRRSARLSAK, RAKLQRRSARLSAK, HARLQRRSARLSAK, RARLQRRFARLFAK, KARLQRRFARLFAK, VARLQRRFARLFAK, GARLQRRFARLFAK, HARLQRRFARLFAK, HAHLQRRFARLFAK, RAKLQRRFARLFAK, RARLQRRLARLLAK, KARLQRRLARLLAK, VARLQRRLARLLAK, GARLQRRLARLLAK, HARLQRRLARLLAK, RAKLQRRLARLLAK, HAHLQRRLARLLAK, RARLQRRSARLSA, KARLQRRSARLSA, VARLQRRSARLSA, HARLQRRSARLSA, GARLQRRSARLSA, RAKLQRRSARLSA, HAHLQRRSARLSA, RARLQRRFARLFA, KARLQRRFARLFA, VARLQRRFARLFA, GARLQRRFARLFA, HARLQRRFARLFA, HAHLQRRFARLFA, RAKLQRRFARLFA, RARLQRRLARLLA, KARLQRRLARLLA, VARLQRRLARLLA, GARLQRRLARLLA, HARLQRRLARLLA, RAKLQRRLARLLA, HAHLQRRLARLLA, RARLQRRSAR, KARLQRRSAR, VARLQRRSAR, HARLQRRSARL, GARLQRRSAR, RAKLQRRSAR, HAHLQRRSAR, RARLQRRSAR, KARLQRRFAR, VARLQRRFAR, GARLQRRFARLFA, HARLQRRFAR, HAHLQRRFAR, RAKLQRRFAR, RARLQRRLAR, KARLQRRLAR, VARLQRRLAR, GARLQRRLAR, HARLQRRLAR, RAKLQRRLAR, HAHLQRRLAR, RGRLQRRSARLSAK, RVRLQRRSARLSAK, RGRLQRRSARLSA, RVRLQRRSARLSA, RGRLQRRSARLS, RVRLQRRSARLS, RGRLQRRSARL, RVRLQRRSARL, RGRLQRRSAR, or RVRLQRRSAR, with one or more amino acid substitutions (e.g., a conservative or non-conservative substitution).

Nucleolin binding moieties and peptides further include antibodies, and antibod_{y} fragments. Exemplary antibodies include anti-nucleolin antibody (NB600-241), epitope 284-709 of human nucleolin (NOVUS Biologicals, Liddleton Colo.), anti-nucleolin antibody (NB100-1920), epitope 1-15 of human nucleolin (NOVUS Biologicals, Liddleton Colo.), anti-nucleolin antibody (NB 100-1920), NB 100-2237, epitope 250 and 300 of human Nucleolin (NOVUS Biologicals, Liddleton Colo.), C23 (H-6): sc-55486 mouse monoclonal IgG₂ₐ, raised against amino acids 271-520 of C23 of human origin (Santa Cruz Biotechnologies, Santa Cruz, Calif.), C23 (D-6): sc-17826 mouse monoclonal IgG₂ₐ; raised against amino acids 271-520 of C23 of human origin, (Santa Cruz Biotechnologies, Santa Cruz, Calif.), C23 (MS-3): sc-8031 mouse monoclonal IgG₁; raised against amino acids 1-706 representing full length C23 of human origin (Santa Cruz Biotechnologies, Santa Cruz, Calif.), C23 (4E2): sc-56640 mouse monoclonal IgG₁, raised against human C23 (Santa Cruz Biotechnologies, Santa Cruz, Calif.), Nucleolin antibody (ab50279), IgG, polyclonal, N terminal domain—epitope amino acids 2-17 (Abeam, Cambridge Mass.), Nucleolin antibody (ab22758), IgG, polyclonal, N terminal domain—amino acids 1-100, Nucleolin antibody (ab14966) IgG1, monoclonal, whole nuclear protein (Abcam, Cambridge Mass.).

An “antibody” refers to any monoclonal or polyclonal immunoglobulin molecule. The term “monoclonal,” when used in reference to an antibody refers to an antibody that is based upon, obtained from or derived from a single clone, including any eukaryotic, prokaryotic, or phage clone. A “monoclonal” antibody is therefore defined herein structurally, and not the method by which it is produced.

Antibodies include polyclonal or monoclonal IgG, IgA, IgM, IgE, IgD, and any subclass thereof. Exemplary subclasses for IgG are IgG₁, IgG₂, IgG₃ and IgG₄. Antibodies include those produced by or expressed on cells, such as B cells.

Antibodies can have kappa or lambda light chain sequences, either full length as in naturally occurring antibodies, mixtures thereof (i.e., fusions of kappa and lambda chain sequences), and subsequences/fragments thereof. Naturally occurring antibody molecules contain two kappa or two lambda light chains. The primary difference between kappa and lambda light chains is in the sequences of the constant region.

An antibody fragment or subsequence refers to a portion of a full length antibody that retains at least partial antigen binding capability of a comparison full length antibody. Exemplary antibody fragments include Fab, Fab′, F(ab′)2, Fv, Fd, single-chain Fv (scFv), disulfide-linked Fvs (sdFv), VL, VH, Camel Ig, V-NAR, VHH, trispecific (Fab3), bispecific (Fab2), diabody ((VL-VH)2 or (VH-VL)2), triabody (trivalent), tetrabody (tetravalent), minibody ((scFV-CH3)2), bispecific single-chain Fv (Bis-scFv), IgGdeltaCH2, scFv-Fc, (scFv)2-Fc, or other antigen binding fragments, such as but not limited to affibodies, aptamers, avimers and nanobodies. Exemplary antibodies bind to epitopes present on nucleolin. Exemplary nucleolin epitopes include 284-709 of human nucleolin, and 1-15, 2-17, 1-100, 250-300, 271-520 or 1-706 of human nucleolin.

Antibodies and fragments or subsequences thereof also include those that compete for binding of a reference antibody as set forth herein. In particular embodiments, an antibody binds to cells expressing nucleolin and competes for binding of one or more antibodies set forth as anti-nucleolin antibody (NB600-241), an antibody that binds to epitope 284-709 of human nucleolin (NOVUS Biologicals, Liddleton Colo.), anti-nucleolin antibody (NB100-1920), an antibody that binds to epitope 1-15 of human nucleolin (NOVUS Biologicals, Liddleton Colo.), anti-nucleolin antibody (NB 100-1920), NB 100-2237, an antibody that binds to epitope 250 and 300 of human Nucleolin (NOVUS Biologicals, Liddleton Colo.), C23 (H-6), sc-55486 mouse monoclonal IgG2a, raised against amino acids 271-520 of C23 of human origin (Santa Cruz Biotechnologies, Santa Cruz, Calif.), C23 (D-6): sc-17826 mouse monoclonal IgG2a; raised against amino acids 271-520 of C23 of human origin, (Santa Cruz Biotechnologies, Santa Cruz, Calif.), C23 (MS-3)_{:} sc-8031 mouse monoclonal IgG1; raised against amino acids 1-706 representing full length C23 of human origin (Santa Cruz Biotechnologies, Santa Cruz, Calif.), C23 (4E2): sc-56640 mouse monoclonal IgG1, raised against human C23 (Santa Cruz Biotechnologies, Santa Cruz, Calif.), nucleolin antibody (ab50279), IgG, polyclonal, an antibody that binds to N terminal domain—epitope amino acids 2-17 (Abeam, Cambridge Mass.), Nucleolin antibody (ab22758), IgG, polyclonal, an antibody that binds to N terminal domain-amino acids 1-100, Nucleolin antibody (ab14966) IgG1, monoclonal, or antibody that binds to whole nuclear protein (Abeam, Cambridge Mass.).

Antibodies and fragments or subsequences thereof also include those that have about the binding affinity of a reference antibody as set forth herein, for example, an affinity within about 1-5,000-fold, greater or less than the binding affinity of a reference antibody. In particular embodiments, the antibody has a binding affinity, within about 1-5,000-fold, greater or less than the binding affinity, of antibody (NB600-241), an antibody that binds to epitope 284-709 of human nucleolin (NOVUS Biologicals, Liddleton CO), anti-nucleolin antibody (NB100-1920), an antibody that binds to epitope 1-15 of human nucleolin (NOVUS Biologicals, Liddleton Colo.), anti-nucleolin antibody (NB100-1920), NB100-2237, an antibody that binds to epitope 250 and 300 of human Nucleolin (NOVUS Biologicals, Liddleton Colo.), C23 (H-6), sc-55486 mouse monoclonal IgG₂ₐ, raised against amino acids 271-520 of C23 of human origin (Santa Cruz Biotechnologies, Santa Cruz, Calif.), C23 (D-6): sc-17826 mouse monoclonal IgG₂ₐ; raised against amino acids 271-520 of C23 of human origin, (Santa Cruz Biotechnologies, Santa Cruz, Calif.), C23 (MS-3): sc-8031 mouse monoclonal IgG₁; raised against amino acids 1-706 representing full length C23 of human origin (Santa Cruz Biotechnologies, Santa Cruz, Calif.), C23 (4E2): sc-56640 mouse monoclonal IgG₁, raised against human C23 (Santa Cruz Biotechnologies, Santa Cruz, Calif.), nucleolin antibody (ab50279), IgG, polyclonal, an antibody that binds to N terminal domain-epitope amino acids 2-17 (Abeam, Cambridge Mass.), Nucleolin antibody (ab22758), IgG, polyclonal, an antibody that binds to N terminal domain-amino acids 1-100, Nucleolin antibody (ab14966) IgG1, monoclonal, or antibody that binds to whole nuclear protein (Abeam, Cambridge Mass.).

Additional specific non-limiting antibodies and fragments thereof have a binding affinity for nucleolin within about K_{d} 10⁻² M to about K_{d} 10⁻¹⁵ M, or within about K_{d} 10⁻⁵ M to about K_{d} 10⁻¹² M. In more particular embodiments, an antibody has binding affinity for nucleolin with a dissociation constant (KD) less than 5×10⁻² M, 10⁻² M, 5×10⁻³ M, 10⁻³ M 5×10⁻⁴ M, 10⁻⁴ M 5×10⁻⁵ M, 10⁻⁵ M 5'10⁻⁶ M, 10⁻⁶ M 5×10⁻⁷ M, 10⁻⁷ M 5×10⁻⁸ M, 10⁻⁸ M 5×10⁻⁹ M, 10⁻⁹ M 5×10⁻¹⁰ M, 10⁻¹⁰ M 5×10⁻¹¹ M, 10⁻¹¹ M 5×10⁻¹² M, 10⁻¹² M 5×10⁻¹³ M, 10⁻¹³ M 5×10⁻¹⁴ M, 10⁻¹⁴ M 5×10⁻¹⁵ M, and 10⁻¹⁵ M.

Binding affinity can be determined by association (Kₐ) and dissociation (K_{d}) rate. Equilibrium affinity constant, KD, is the ratio of Kₐ/K_{d}. Association (Kₐ) and dissociation (K_{d}) rates can be measured using surface plasmon resonance (SPR) (Rich and Myszka, Curr. Opin. Biotechnol. 11:54 (2000); Englebienne, Analyst. 123:1599 (1998)). Instrumentation and methods for real time detection and monitoring of binding rates are known and are commercially available (BiaCore 2000, Biacore AB, Upsala, Sweden; and Malmqvist, Biochem. Soc. Trans. 27:335 (1999)). KD values can be defined as the antibody concentration required to saturate one half (50%) of the binding sites on nucleolin.

Methods of producing polyclonal and monoclonal antibodies are known in the art. For example, nucleolin or a subsequence thereof, or an immunogenic fragment thereof, optionally conjugated to a carrier such as keyhole limpet hemocyanin (KLH) or ovalbumin (e.g., BSA), or mixed with an adjuvant such as Freund's complete or incomplete adjuvant, and used to immunize an animal. Using conventional hybridoma technology, spleenocytes from immunized animals that respond to nucleolin can be isolated and fused with myeloma cells. Monoclonal antibodies produced by the hybridomas can be screened for reactivity with nucleolin or an immunogenic fragment thereof.

Animals that may be immunized include mice, rats, rabbits, goats, sheep, cows or steer, guinea pigs or primates. Initial and any optional subsequent immunization may be through intravenous, intraperitoneal, intramuscular, or subcutaneous routes. Subsequent immunizations may be at the same or at different concentrations of nucleolin, or a subsequence thereof, and may be at regular or irregular intervals.

Animals include those genetically modified to include human Ig (e.g., IgG) gene loci, which can therefore be used to produce human antibodies. Transgenic animals with one or more human immunoglobulin genes that do not express endogenous immunoglobulins are described, for example in, U.S. Pat. No. 5,939,598. Additional methods for producing human polyclonal antibodies and human monoclonal antibodies are described (see, e.g., Kuroiwa et al., Nat. Biotechnol. 20:889 (2002); WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598). An overview of the technology for producing human antibodies is described in Lonberg and Huszar (Int. Rev. Immunol. 13:65 (1995)).

Antibodies can also be generated using other techniques including hybridoma, recombinant, and phage display technologies, or a combination thereof (see U.S. Pat. Nos. 4,902,614, 4,543,439, and 4,411,993; see, also Monoclonal Antibodies, Hybridomas: A New Dimension in Biological Analyses, Plenum Press, Kennett, McKearn, and Bechtol (eds.), 1980, and Harlow et al., Antibodies: A Laboratory Manual, Cold Spring Harbor Laboratory Press, 2nd ed. 1988).

Antibodies can be humanized using a variety of techniques known in the art including, for example, CDR-grafting (EP 239,400; WO91/09967; U.S. Pat. Nos. 5,225,539; 5,530,101; and 5,585,089), veneering or resurfacing (EP 592,106; EP 519,596; Padlan, Molecular Immunol. 28:489 (1991); Studnicka et al., Protein Engineering 7:805 (1994); Roguska. et al., Proc. Nat'l. Acad. Sci. USA 91:969 (1994)), and chain shuffling (U.S. Pat. No. 5,565,332). Human consensus sequences (Padlan, Mol. Immunol. 31:169 (1994); and Padlan, Mol. Immunol. 28:489 (1991)) have previously used to produce humanized antibodies (Carter et al., Proc. Natl. Acad. Sci. USA 89:4285 (1992); and Presta et al., J. Immunol. 151:2623 (1993)).

Methods for producing chimeric antibodies are known in the art (e.g., Morrison, Science 229:1202 (1985); Oi et al., BioTechniques 4:214 (1986); Gillies et al., J. Immunol. Methods 125:191 (1989); and U.S. Pat. Nos. 5,807,715; 4,816,567; and 4,816,397). Chimeric antibodies in which a variable domain from an antibody of one species is substituted for the variable domain of another species are described, for example, in Munro, Nature 312:597 (1984); Neuberger et al., Nature 312:604 (1984); Sharon et al., Nature 309:364 (1984); Morrison et al., Proc. Nat'l. Acad. Sci. USA 81:6851 (1984); Boulianne et al., Nature 312:643 (1984); Capon et al., Nature 337:525 (1989); and Traunecker et al., Nature 339:68 (1989).

Antibody subsequences and fragments can be prepared by proteolytic hydrolysis of the antibody, for example, by pepsin or papain digestion of whole antibodies. Antibody subsequences and fragments produced by enzymatic cleavage with pepsin provide a 5S fragment denoted F(ab′)₂. This fragment can be further cleaved using a thiol reducing agent to produce 3.5S Fab' monovalent fragments. Alternatively, an enzymatic cleavage using pepsin produces two monovalent Fab' fragments and the Fe fragment directly (see, e.g., U.S. Pat. Nos. 4,036,945 and 4,331,647; and Edelman et al., Methods Enymol. 1:422 (1967)). Other methods of cleaving antibodies, such as separation of heavy chains to form monovalent light-heavy chain fragments, further cleavage of fragments, or other enzymatic or chemical may also be used.

Suitable purification techniques that additionally may be employed in antibody methods include affinity purification such as purification of protein A or G column, non-denaturing gel purification, HPLC or RP-HPLC, size exclusion,or any combination of these techniques.

Nucleolin binding peptides and fusion constructs (peptide/lytic domain constructs, antibody/peptide constructs and antibody/lytic domain constructs) include peptides and fusion constructs that are cytotoxic to cells. As used herein, the term “cytotoxicity” and grammatical variations thereof, means measurable or detectable toxicity towards or against a cell (typically a eukaryotic cell). Toxicity is such that cell proliferation or viability is reduced, inhibited, decreased or prevented. Toxicity can, but need not, result in cell death, lysis, or apoptosis. Thus, the term “cytotoxicity” does not require that the cells be killed, lysed or die. Rather, the term means a detrimental effect on the health of a cell, which can be measured by various indicators of cell health, such as cell proliferation rate or frequency, cell viability or survivability, morphological features characteristic of poor cell health, increased sensitivity of a cell to environmental changes or physical stimuli, etc.

Nucleolin binding peptides and fusion constructs include sequences in which all or a part of the sequence includes an alpha helical structure. A nucleolin-binding peptide or fusion construct can contain a continuous alpha helical structure that spans at least 30% of the length of the peptide. Thus, if a sequence is 20 amino acid residues in length, at least 6 continuous amino acid residues within the sequence are in an alpha helical structure. A nucleolin binding peptide or fusion construct can contain a continuous alpha helical structure that spans 30-40%, 40-50%, 50-60%, 60-70%, 70-80%, 80-90%, of 90-100% residues of the length of the peptide, such as an invention peptide or a lytic domain.

Nucleolin binding peptides and fusion constructs of the invention also include sequences in which all or a part of the sequence is amphipathic. Nucleolin binding peptides and fusion constructs of the invention further include sequences in which all or a part of the sequence is cationic. Such sequences may also form an alpha helical structure, at least over a portion of the full length sequence, which may or may not include a nucleolin binding peptide or a cytotoxic moiety or domain such as in a fusion construct.

Peptides of the invention do not require actively dividing or proliferating cells in order to stimulate, induce or promote lysis or death of target cells. Furthermore, the peptides are not likely to be immunogenic as they can be made to be relatively small in size. In addition, the peptides can kill multi-drug resistant cells.

Moreover, the peptides of the invention can have low hemolytic activity (HA₅₀). For example, hemolytic activity against human red blood cells can be more than about 500 μM, or more than about 250 μM, or more than about 100 μM, or more than about 75 μM, or more than about 50 μM, or more than about 25 μ, or more than about 10 μM, or more than about 5 μM, or not be detectable.

Nucleolin binding peptides and antibodies can be linked or conjugated to one or more additional entities, domains or moieties. Such sequences are referred to as fusion or chimeric constructs or sequences, since they are covalently or non-covalently associated or bound to each other.

As used herein, the term “fusion” or “chimeric” and grammatical variations thereof, when used in reference to a construct or sequence, means that the construct contains portions or sections that are derived from, obtained or isolated from, or are based upon or modeled after two different molecular entities that are distinct from each other and do not typically exist combined together in nature. That is, for example, one portion of the fusion construct includes or consists of a peptide or antibody that binds to nucleolin and a second portion of the fusion construct includes or consists of another entity, domain or moiety, such as a moiety that has cytotoxicity, each of first and second portions are distinct and do not typically exist combined together in nature. A fusion construct can also be referred to as a “conjugate,” wherein the conjugate includes or consists of a first nucleolin binding portion and a second domain or another moiety.

Non-limiting examples of fusion constructs include a nucleolin binding peptide or antibody linked or conjugated (fused) to a detectable agent or a tag. Such fusion constructs can be used to target for detection or screening of nucleolin expressing cells. Another non-limiting example of fusion constructs include a nucleolin binding peptide or antibody linked or conjugated (fused) to a cytotoxic domain (e.g., a lytic peptide). Such fusion constructs can be used to target nucleolin expressing cells for lysis or death.

Non-limiting examples of cytotoxic domains include an amino acid sequence, chemotherapeutic drug, a radionuclide, bacterial toxin or bacterial toxin fragment, a pore-forming peptide, or a toxin. Exemplary cytotoxic (lytic) domains joined to a nucleolin binding domain include or consists of an amino acid sequence selected from KFAKFAKKFAKFAKK, KFAKFAKKFAKFAKKF, KFAKFAKKFAKFAKKFA, KFAKFAKKFAKFAKKFAK, KFAKFAKKFAKFAKKFAKF and KFAKFAKKFAKFAKKFAKFA; or an amino acid sequence selected from KFAKFAKKFAKFAKK, KFAKFAKKFAKFAKKF, KFAKFAKKFAKFAKKFA, KFAKFAKKFAKFAKKFAK, KFAKFAKKFAKFAKKFAKF and KFAKFAKKFAKFAKKFAKFA having one or more of the K residues substituted with any of an F or L residue, one or more of the F residues substituted with any of a K, A or L residue, or one or more of the A residues substituted with any of a K, F or L residue; or include or consist of a 12 to 28 amino acid sequence that includes a peptide selected from KFAKFAKKFAKFAKK, KFAKFAKKFAKFAKKF, KFAKFAKKFAKFAKKFA, KFAKFAKKFAKFAKKFAK, KFAKFAKKFAKFAKKFAKF and KFAKFAKKFAKFAKKFAKFA, or a 12 to 28 amino acid sequence that includes a peptide selected from KFAKFAKKFAKFAKK, KFAKFAKKFAKFAKKF, KFAKFAKKFAKFAKKFA, KFAKFAKKFAKFAKKFAK, KFAKFAKKFAKFAKKFAKF and KFAKFAKKFAKFAKKFAKFA having one or more of the K residues substituted with any of an F or L residue, one or more of the F residues substituted with any of a K, A or L residue, or one or more of the A residues substituted with any of a K, F or L residue. Additional exemplary non-limiting examples of lytic domains are described, for example, in U.S. Pat. No. 6,635,740, which is incorporated herein by reference.

An additional non-limiting example of cytotoxic (lytic) domains that can be joined to a nucleolin binding peptide or antibody include or consists of amino acid sequences of anti-microbial defense peptides, including those of insects, amphibians, and mammals. Such peptides include all or a portion of a sequence of a cecropin (e.g., cecropin A, cecropin B, cecropin D), defensin, melittin, sarcotoxin, or magainin peptide, and analogs and hybrids (chimeras) of such peptides. Such peptides can be 23-39 amino acids, or larger or smaller, and may form an amphipathic alpha-helix. A specific example, of a peptide includes, for example, a sequence including or consisting of a FALALKALKKALKKLKKALKKAL, or a subsequence thereof.

Additional examples of these and other peptides are described, for example, in U.S. Pat. Nos. 6,635,740 and 5,789,542; WO 98/12866; WO 93/01723; WO 89/00199; and WO 90/12866; Boman et al., Curr. Top. Microbiol. Immunol. 94/95:75 (1981); Boman et al., Annu. Rev. Microbiol. 41:103 (1987); Zasloff, Proc. Natl. Acad. Sci. USA, 84:3628 (1987); Ganz et al., J. Chin. Invest. 76: 1427 (1985); and Lee et al., Proc. Natl. Acad. Sci. USA, 86:9159 (1989). Still additional lytic peptide sequences are described in Yamada et al., Biochem. J., 272:633 (1990); Taniai et al., Biochimica Et Biophysica Acta. 1132: 203 (1992); Boman et al., Febs Letters 259:103 (1989); Tessier et al., Gene 98:177 (1991); Blondelle et al., Biochemistry 30:4671(1991); Andreu et al., Febs Letters 296:190 (1992); Macias et al., Can. J. Microbiol. 36:582 (1990); Rana et al., Biochemistry 30:5858 (1991); Diamond et al., Proc. Natl. Acad. Sci. USA 90:4596 ff (1993); Selsted et al., J. Biol. Chem. 268:6641 ff (1993); Tang et al., J. Biol. Chem. 268:6649 ff (1993); Lehrer et al., Blood 76:2169 (1990); Ganz et al., Sem. Resp. Infect. 1:107 (1986); Kagan et al., Proc. Natl. Acad. Sci. USA 87:210 (1990); Wade et al., Proc. Natl. Acad. Sci. USA 87:4761 (1990); Romeo et al., J. Biol. Chem. 263:9573 (1988); Hultmark et al., Eur. J. Biochem. 106:7 (1980); Hultmark et al., Eur. J. Biochem. 127:207 (1982); Andrequ et al., Biochem. 24:1683 (1985). Boman et al., Eur. J. Biochem. 201:23 (1991); Lee et al., Proc. Natl. Acad. Sci. USA. 86:9159 (1989); Jaynes et al., FASEB. 2878 (1988); Arrowood et al., J. Protozool., 38:161S (1991); Arrowood et al., Antimicrob. Agents Chemother., 35:224 (1991); Jaynes et al., Peptide Research, 2:1-5 (1989); Reed et al., Mol. Reprod. Devel., 31:106 (1992).

Defensins are typically small peptides containing six to eight cysteine residues. Examples of such peptides are described, for example, in Ganz et al., J. Clin. Invest., 76:1427 (1985), human neutrophil peptides HNP-1, and HNP-3, Dimarcq et al., EMBO J., 9:2507 (1990); and Fisher et al., Proc. Natl. Acad. Sci. USA, 84:3628 (1987).

Sarcotoxins are typically slightly larger, and have been purified from the fleshfly Sarcophaga peregrina. Examples of these peptides are described, for example, in Okada et al., J. Biol. Chem., 260:7174 (1985).

Other lytic peptides have been found in amphibians. Examples of these peptides are described, for example, in Gibson et al., J. Biol. Chem., 261:5341(1986); and Givannini et al., Biochem. J., 243:113 (1987), which describe two peptides from Xenopus laevis, peptides named PGS and Gly¹⁰ Lys²² PGS. Xenopus-derived peptides have been reported to have antimicrobial activity, and are also referred to as magainins (Zasloff, Proc. Natl. Acad. Sci. USA, 84:3628 (1987)). Morvan et al. (Mol. Mar. Biol., 3:327 (1994)) report in vitro use of a magainin to selectively reduce viability of the parasite Bonamia ostreae at doses that did not affect cells of the flat oyster Ostrea edulis.

Cecropins target pathogens or compromised cells. A synthetic lytic peptide known as S-1 (or Shiva 1) has been reported to destroy intracellular Brucella abortus-, Trypanosoma cruzi-, Cryptosporidium parvum-, and infectious bovine herpes virus I (IBR)-infected host cells, with little or no toxic effects on noninfected mammalian cells (Jaynes et al., Peptide Research 2:1 (1989); Wood et al., Proc. Ann. Amer. Soc. Anim. Sci., Utah State University, Logan, Utah J. Anim. Sci., 65:380S (1987); Arrowood et al., J. Protozoal. 38:161S (1991); Arrowood et al., Antimicrob. Agents Chemother. 35:224 (1991); and Reed et al., Mol. Reprod. Devel., 31:106 (1992)).

Different classes of lytic peptides have a positively charged, amphipathic sequence containing at least 20 amino acids, and may contribute to lytic activity in some peptides. A report of structure-activity relationship of Lepidopteran, a self-defense peptide of Bombyx is in Shiba et al. (Tetrahedron 44:787 (1988)).

Examples of such peptides are those designated D1A21(FAFAFKAFKKAFKKFKKAFKKAF), D2A21(FAKKFAKKFKKFAKKFAKFAFAF), D5C (KRKRAVKRVGRRLKKLARKIARLGVAF), and D5C1 (KRKRAVKRVGRRLKKLARKIARLGVAKLAGLRAVLKF). These and other lytic peptides are suitable to join to a nucleolin binding peptide.

Fusion constructs that include a nucleolin binding domain, region or portion at the amino-terminus include a second moiety or domain, such as a cytotxic domain, at the carboxyl-terminus. Fusion constructs that include a nucleolin binding peptide, region or portion at the carboxyl-terminus include a second moiety or domain, such as a cytotoxic domain at the amino-terminus. Where additional domains are present (e.g., third, fourth, fifth, sixth, seventh, etc. domains), such domains can be positioned at the NH₂-terminus or carboxyl terminus of a nucleolin binding peptide, region or portion or the second domain, such as the cytotoxic domain.

Nucleolin binding peptides and fusion constructs include or consist of amino acid sequences (peptides, polypeptides, proteins, lectins), nucleic acids (DNA, RNA) and carbohydrates (saccharides, sialic acid, galactose, mannose, fucose, acetylneuraminic acid, etc.). The terms “amino acid sequence,” “protein,” “polypeptide” and “peptide” are used interchangeably herein to refer to two or more amino acids, or “residues,” covalently linked by an amide bond or equivalent Amino acid sequences can be linked by non-natural and non-amide chemical bonds including, for example, those formed with glutaraldehyde, N-hydroxysuccinimide esters, bifunctional maleimides, or N,N′-dicyclohexylcarbodiimide (DCC). Non-amide bonds include, for example, ketomethylene, aminomethylene, olefin, ether, thioether and the like (see, e.g., Spatola in: Chemistry and Biochemistry of Amino Acids, Peptides and Proteins, Vol. 7, pp 267-357 (1983), “Peptide and Backbone Modifications,” eds. Marcel Decker, NY). Nucleolin binding peptides and fusion constructs include L-amino acid sequences, D-amino acid sequences and amino acid sequences with mixtures of L-amino acids and D-amino acids. Amino acid sequences of nucleolin binding peptides and fusion constructs can be a linear or a cyclic structure, conjugated to a distinct entity or moiety (e.g., third, fourth, fifth, sixth, seventh, etc. domains), form intra or intermolecular disulfide bonds, and also form higher order multimers or oligomers with the same or different amino acid sequence, or other molecules.

Exemplary lengths of nucleolin binding peptides and fusion constructs are from about 5 to 15, 20 to 25, 25 to 50, 50 to 100, 100 to 150, 150 to 200, or 200 to 300 or more amino acid residues in length (or any length within such ranges or a combination of such ranges). In various particular embodiments, a nucleolin binding peptide or fusion construct includes or consists of an amino acid sequence of about 1 to 10, 10 to 20, 15 to 20, 20 to 30, 30 to 40, 40 to 50, 60 to 70, 70 to 80, 80 to 90, 90 to 100 or more residues (or any length within such ranges or a combination of such ranges). In more particular embodiments, a nucleolin binding peptide or fusion construct consists of a 10 to 31 residue amino acid sequence.

An amphipathic alpha-helix contains mostly hydrophilic amino acids on one side of the alpha-helix and the other side contains mostly hydrophobic amino acids. Since the alpha helix makes a complete turn for every 3.6 residues, the amino acid sequence of an amphipathic alpha helix alternates between hydrophilic and hydrophobic residues every 3 to 4 residues. A PNNPNNP repeat pattern or motif is predicted to form an amphipathic alpha-helix where P represents a positively charged amino acid residue and N a neutral amino acid residue. A PNNPNNP repeat pattern provides a cationic binding site for the lytic peptide to a negatively charged cell membrane and a hydrophobic site for membrane interaction/penetration. Peptides and fusion constructs therefore include those with one or more uninterrupted PNNPNNP repeat patterns or motifs, or one or more interrupted PNNPNNP repeat patterns or motifs, which can form an amphipathic alpha-helix.

As used herein, the term “subsequence” or “fragment” means a portion of the full length molecule. A subsequence of an invention peptide, nucleolin, a lytic domain or antibody has one or more fewer amino acids than a full length invention peptide, nucleolin, a lytic domain or antibody (e.g., heavy or light chain sequence) (e.g. one or more internal or terminal amino acid deletions from either amino or carboxy-termini). Subsequences therefore can be any length up to the full length native molecule.

Subsequences and amino acid substitutions of the various nucleolin binding peptides, antibodies and fusion constructs set forth herein, are also included. In particular embodiments, a subsequence of a nucleolin binding peptide, antibody or fusion constructs has at least 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100 or more amino acid residues (or any length within such ranges or a combination of such ranges.

The invention therefore includes modifications or variations, such as substitutions, additions or deletions of nucleolin binding peptides and fusion constructs. Thus, a nucleolin binding peptide or fusion construct that includes a peptide sequence can incorporate any number of conservative or non-conservative amino acid substitutions, as long as such substitutions do not destroy activity (e.g., nucleolin binding or cytotoxic activity). Thus, for example, a modified nucleolin binding peptide can retain at least partial binding activity, and a cytotoxic domain can retail at least partial cell lysis, killing or apoptosis. Particular non-limiting examples are amino acid substitutions to produce or increase the length of a helical region in a peptide or fusion construct.

Modified proteins include one or more D-amino acids substituted for L-amino acids (and mixtures thereof), structural and functional analogues, for example, peptidomimetics having synthetic or non-natural amino acids or amino acid analogues and derivatized forms. Modifications include cyclic structures such as an end-to-end amide bond between the amino and carboxy-terminus of the molecule or intra- or inter-molecular disulfide bond.

Modified proteins further include amino acid substitutions. In particular embodiments, a modified protein has one or a few conservative or non-conservative substitutions. Such proteins that include amino acid substitutions can be encoded by a nucleic acid. Consequently, nucleic acid sequences encoding proteins that include amino acid substitutions are also provided.

A “conservative substitution” is a replacement of one amino acid by a biologically, chemically or structurally similar residue. Biologically similar means that the substitution is compatible with a biological activity, e.g., lytic activity. Structurally similar means that the amino acids have side chains with similar length, such as alanine, glycine and serine, or having similar size, or the structure of a first, second or additional domain is maintained, such as an amphipathic alpha helix. Chemical similarity means that the residues have the same charge or are both hydrophilic or hydrophobic. Particular examples include the substitution of one hydrophobic residue, such as isoleucine, valine, leucine or methionine for another, or the substitution of one polar residue for another, such as the substitution of arginine for lysine, glutamic for aspartic acids, or glutamine for asparagine, serine for threonine, etc. Routine assays can be used to determine whether a nucleolin binding peptide or fusion construct variant has activity, e.g., nucleolin binding activity or cytotoxic activity.

Specific examples include a substitution or deletion of one or more amino acid (e.g., 1-3, 3-5, 5-10, 10-20, or more) residues of a nucleolin binding peptide or fusion construct. A modified nucleolin binding peptide or fusion construct can have a peptide sequence with 50%, 60%, 70%, 75%, 80%, 85%, 90%, 95%, 96%, 97%, 98%, or more identity to a reference sequence (e.g., a nucleolin binding peptide or fusion construct as set forth herein).

The term “identity” and “homology” and grammatical variations thereof mean that two or more referenced entities are the same. Thus, where two amino acid sequences are identical, they have the same amino acid sequence, or are 100% identical or homologus. “Areas, regions or domains of identity” mean that a portion of two or more referenced entities are the same. Thus, where two amino acid sequences are identical or homologous over one or more sequence regions, they share identity in these regions. The term “complementary,” when used in reference to a nucleic acid sequence means the referenced regions are 100% complementary, i.e., exhibit 100% base pairing with no mismatches.

Due to variation in the amount of sequence conservation between structurally and functionally related proteins, the amount of sequence identity required to retain a function or activity (e.g., nucleolin binding or cytotoxicity) depends upon the protein, the region and the function or activity of that region. As disclosed herein a variety of nucleolin binding peptides and fusion constructs that retain at least partial nucleolin binding or cytotoxic activity or function are provided and variations thereof are known to one of skill in the art in view of the guidance herein.

The extent of identity between two sequences can be ascertained using a computer program and mathematical algorithm known in the art. Such algorithms that calculate percent sequence identity (homology) generally account for sequence gaps and mismatches over the comparison region. For example, a BLAST (e.g., BLAST 2.0) search algorithm (see, e.g., Altschul et al., J. Mol. Biol. 215:403 (1990), publicly available through NCBI) has exemplary search parameters as follows: Mismatch −2; gap open 5; gap extension 2. For polypeptide sequence comparisons, a BLASTP algorithm is typically used in combination with a scoring matrix, such as PAM100, PAM 250, BLOSUM 62 or BLOSUM 50. FASTA (e.g., FASTA2 and FASTA3) and SSEARCH sequence comparison programs are also used to quantitate the extent of identity (Pearson et al., Proc. Natl. Acad. Sci. USA 85:2444 (1988); Pearson, Methods Mol Biol. 132:185 (2000); and Smith et al. J. Mol. Biol. 147:195 (1981)). Programs for quantitating protein structural similarity using Delaunay-based topological mapping have also been developed (Bostick et al., Biochem Biophys Res Commun. 304:320 (2003)).

Individual residues of nucleolin binding peptides and fusion constructs and additional domains can be joined by a covalent or a non-covalent bond. Non-limiting examples of covalent bonds are amide bonds, non-natural and non-amide chemical bonds, which include, for example, glutaraldehyde, N-hydroxysuccinimide esters, bifunctional maleimides, N,N′-dicyclohexylcarbodiimide (DCC) or N,N′-diisopropylcarbodiimide (DIC). Linking groups alternative to amide bonds include, for example, ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola in:Chemistry and Biochemistry of Amino Acids, Peptides and Proteins, Vol. 7, pp 267-357 (1983), “Peptide and Backbone Modifications,” eds, Marcel Decker, NY).

As disclosed herein, nucleolin binding peptides can be fused or joined immediately adjacent to another domain by a covalent or a non-covalent bond. Nucleolin binding peptides can be separated by an intervening region, such as a hinge, spacer or linker positioned between the peptide and a second domain (e.g., cytotoxic domain). In one embodiment, a nucleolin binding peptide and a second domain are joined by a carbon chain. Multi-carbon chains include carboxylic acids (e.g., dicarboxylic acids) such as glutaric acid, succinic acid and adipic acid.

In another embodiment, a nucleolin binding peptide and second domain are joined by an amino acid, peptide or a non-peptide hinge, spacer or linker positioned between the nucleolin binding and second domains. Peptide hinge, spacer or linker sequences can be any length, but typically range from about 1-10, 10-20, 20-30, 30-40, or 40-50 amino acid residues. In particular embodiments, a peptide hinge, spacer or linker positioned between a first and second domain is from 1 to 25 L- or D-amino acid residues, or 1 to 6 L- or D-amino acid residues. Particular amino acid residues that are included in sequences positioned between the nucleolin binding and second domains include one or more of or C, A, S or G amino acid residues. Specific non-limiting examples of peptides positioned between the nucleolin binding and second domains include a sequence within or set forth as: GSGGS, ASAAS, or aliphatic carbon chain (e.g., CCCCC). Derivatives of amino acids and peptides can be positioned between the nucleolin binding and second domain. A specific non-limiting example of an amino acid derivative is a lysine derivative, or a 6 carbon linker such as a-amino-caproic acid.

Nucleolin binding peptides and fusion constructs with or without a hinge, spacer or linker, or a third, fourth, fifth, sixth, seventh, etc. domain can be entirely composed of natural amino acids or synthetic, non-natural amino acids or amino acid analogues, or can include derivatized forms. Nucleolin binding and fusion constructs can include one or more D-amino acids substituted for L-amino acids, mixtures of D-amino acids and L-amino acids, or a sequence composed entirely of D-amino acid residues.

Nucleolin binding peptides and fusion construct in vivo half-life can be increased using one or more non-naturally occurring amino acids or derivatives, for example, D-amino acids (e.g., up to 30% or more of all residues are D-enantiomers) are resistant to serum proteolysis and therefore can be active for longer times thereby increasing in vivo potency. Furthermore, constructing nucleolin binding peptides or fusion constructs with one or more non-naturally occurring amino acids or derivatives can reduce hemolytic activity. Such nucleolin binding peptides and fusion constructs with D-enantiomers also have a greater tendency to be monomeric in solution—they do not significantly aggregate.

Nucleolin binding peptides and fusion constructs can contain any combination of non-natural structural components, which are typically from three structural groups: a) residue linkage groups other than the natural amide bond (“peptide bond”) linkages; b) non-natural residues in place of naturally occurring amino acid residues; or e) residues which induce secondary structural mimicry, i.e., induce or stabilize a secondary structure, e.g., an alpha helix conformation. Nucleolin binding peptides and fusion constructs include cyclic structures such as an end-to-end amide bond between the amino and carboxy-terminus of the molecule or intra- or inter-molecular disulfide bond(s). Nucleolin binding peptides and fusion constructs may be modified in vitro or in vivo, e.g., post-translationally modified to include, for example, sugar or carbohydrate residues, phosphate groups, fatty acids, lipids, etc.

Specific examples of an addition include a third, fourth, fifth, sixth or seventh domain. Nucleolin binding peptides and fusion constructs with another domain therefore include one or more additional domains (third, fourth, fifth, sixth, seventh, etc.) covalently linked thereto to impart a distinct or complementary function or activity. Exemplary additional domains include domains facilitating detection or isolation, which include, for example, metal chelating peptides such as polyhistidine tracts and histidine-tryptophan modules that allow purification on immobilized metals; protein A domains that allow purification on immobilized immunoglobulin; and domain utilized in the FLAGS extension/affinity purification system (Immunex Corp, Seattle Wash.). Optional inclusion of a cleavable sequence such as Factor Xa or enterokinase between a purification domain and the nucleolin binding peptides and fusion constructs can be used to facilitate purification. For example, an expression vector can include a fusion construct-encoding nucleic acid sequence linked to six histidine residues followed by a thioredoxin and an enterokinase cleavage site. The histidine residues facilitate detection and purification of the fusion construct while the enterokinase cleavage site provides a means for purifying the construct from the remainder of the protein (see e.g., Kroll, DNA Cell. Biol. 12:441 (1993)).

Nucleolin binding peptide and fusion construct activity can be affected by various factors and therefore fusion constructs can be designed or optimized by taking into consideration one or more of these factors. Such factors include, for example, length of a fusion construct, which can affect toxicity to cells. In particular, increased length of a continuous helical structure will likely increase cytotoxicity. Cell killing activity of alpha helix forming peptide sequences can also depend on the stability of the helix. Hinge and spacers can affect membrane interaction of a binding domain and helical structure of a peptide lytic domain. For example, shorter fusion constructs, such as constructs less than 21 amino acids that optionally include a spacer or hinge, can exhibit increased cytotoxicity due to increased helix length or stability. The charge of peptides, which is determined in part by the particular amino acid residues present in the domain, also affects cell killing potency. The positioning of the nucleolin binding peptide relative to a cytotoxic domain (N- or C-terminus) also can affect cell killing activity of fusion constructs.

Peptides and peptidomimetics can be produced and isolated using methods known in the art. Peptides can be synthesized, whole or in part, using chemical methods known in the art (see, e.g., Caruthers Nucleic Acids Res. Symp. Ser. 215 (1980); and Banga, A. K., Therapeutic Peptides and Proteins, Formulation, Processing and Delivery Systems (1995), eds., Technomic Publishing Co., Lancaster, Pa.). Peptide synthesis can be performed using various solid-phase techniques (see, e.g., Roberge, Science 269:202 (1995); Merrifield, Methods Enzymol. 289:3(1997)) and automated synthesis may be achieved, e.g., using the ABI 431A Peptide Synthesizer (Perkin Elmer) in accordance with the manufacturer's instructions. Peptides and peptidomimetics can also be synthesized using combinatorial methodologies. Synthetic residues and polypeptides incorporating mimetics can be synthesized using a variety of procedures and methodologies known in the art (see, e.g., Organic Syntheses Collective Volumes, Gilman, et al., eds., John Wiley & Sons, Inc., NY). Modified peptides can be produced by chemical modification methods (see, for example, Belousov, Nucleic Acids Res. 25:3440 (1997); Frenkel, Free Radic. Biol. Med. 19:373 (1995); and Blommers, Biochemistry 33:7886 (1994).

The invention further provides nucleic acids encoding the nucleolin binding peptides and fusion constructs of the invention, and vectors that include nucleic acid that encodes the nucleolin binding peptides. In a particular embodiment, a nucleic acid encodes a continuous amino acid sequence with the general formula, X1-X2-X3-X4-X5-X6-X7-X8-X9-X10-X11-X12-X13-X14, wherein: X1 is R, H, G, K or V; X2 is A, G or V; X3 is R, K or H; X4 is L; X5 is Q; X6 is R; X7 is R; X8 is S, F or L; X9 is A; X10 is R; X11 is L or nothing (absent); X12 is S, F or L or nothing (absent); X13 is A or nothing (absent); X14 is K or nothing (absent); and the peptide binds to nucleolin. In a further embodiment, a nucleic acid encodes a peptide including a sequence set forth as: RARLQRRSARLSAK, KARLQRRSARLSAK, VARLQRRSARLSAK, HARLQRRSARLSAK, GARLQRRSARLSAK, RAKLQRRSARLSAK, HAHLQRRSARLSAK, RARLQRRFARLFAK, KARLQRRFARLFAK, VARLQRRFARLFAK, GARLQRRFARLFAK, HARLQRRFARLFAK, HARLQRRFARLFAK, RAKLQRRFARLFAK, RARLQRRLARLLAK, KARLQRRLARLLAK, VARLQRRLARLLAK, GARLQRRLARLLAK, HARLQRRLARLLAK, RAKLQRRLARLLAK, HAHLQRRLARLLAK, RARLQRRSARLSA, KARLQRRSARLSA, VARLQRRSARLSA, HARLQRRSARLSA, GARLQRRSARLSA, RAKLQRRSARLSA, HAHLQRRSARLSA, RARLQRRFARLFA, KARLQRRFARLFA, VARLQRRFARLFA, GARLQRRFARLFA, HARLQRRFARLFA, HAHLQRRFARLFA, RAKLQRRFARLFA, RARLQRRLARLLA, KARLQRRLARLLA, VARLQRRLARLLA, GARLQRRLARLLA, HARLQRRLARLLA, RAKLQRRLARLLA, HAHLQRRLARLLA, RARLQRRSAR, KARLQRRSAR, VARLQRRSAR, HARLQRRSARL, GARLQRRSAR, RAKLQRRSAR, HAHLQRRSAR, RARLQRRFAR, KARLQRRFAR, VARLQRRFAR, GARLQRRFARLFA, HARLQRRFAR, HAHLQRRFAR, RAKLQRRFAR, RARLQRRLAR, KARLQRRLAR, VARLQRRLAR, GARLQRRLAR, HARLQRRLAR, RAKLQRRLAR, HAHLQRRLAR, RGRLQRRSARLSAK, RARLQRRSARLSAK, RGRLQRRSARLSA, RVRLQRRSARLSA, RGRLQRRSARLS, RVRLQRRSARLS, RGRLQRRSARL, RVRLQRRSARL, RGRLQRRSAR, or RVRLQRRSAR,), and the peptide binds to nucleolin. In further embodiments, nucleic acid encodes a sequence set forth as: RARLQRRSARLSAK, KARLQRRSARLSAK, VARLQRRSARLSAK, HARLQRRSARLSAK, GARLQRRSARLSAK, RAKLQRRSARLSAK, HAHLQRRSARLSAK, RARLQRRFARLFAK, KARLQRRFARLFAK, VARLQRRFARLFAK, GARLQRRFARLFAK, HARLQRRFARLFAK, HAHLQRRFARLFAK, RAKLQRRFARLFAK, RARLQRRLARLLAK, KARLQRRLARLLAK, VARLQRRLARLLAK, GARLQRRLARLLAK, HARLQRRLARLLAK, RAKLQRRLARLLAK, HAHLQRRLARLLAK, RARLQRRSARLSA, KARLQRRSARLSA, VARLQRRSARLSA, HARLQRRSARLSA, GARLQRRSARLSA, RAKLQRRSARLSA, HAHLQRRSARLSA, RARLQRRFARLFA, KARLQRRFARLFA, VARLQRRFARLFA, GARLQRRFARLFA, HARLQRRFARLFA, HAHLQRRFARLFA, RAKLQRRFARLFA, RARLQRRLARLLA, KARLQRRLARLLA, VARLQRRLARLLA, GARLQRRLARLLA, HARLQRRLARLLA, RAKLQRRLARLLA, HAHLQRRLARLLA, RARLQRRSAR, KARLQRRSAR, VARLQRRSAR, HARLQRRSARL, GARLQRRSAR, RAKLQRRSAR, HAHLQRRSAR, RARLQRRFAR, KARLQRRFAR, VARLQRRFAR, GARLQRRFARLFA, HARLQRRFAR, HAHLQRRFAR, RAKLQRRFAR, RARLQRRLAR, KARLQRRLAR, VARLQRRLAR, GARLQRRLAR, HARLQRRLAR, RAKLQRRLAR, HAHLQRRLAR, RGRLQRRSARLSAK, RVRLQRRSARLSAK, RGRLQRRSARLSA, RVRLQRRSARLSA, RGRLQRRSARLS, RVRLQRRSARLS, RGRLQRRSARL, RVRLQRRSARL, RGRLQRRSAR, or RVRLQRRSAR, with one or more amino acid substitutions (e.g., a conservative or non-conservative substitution), and the peptide binds to nucleolin.

Nucleic acid, which can also be referred to herein as a gene, polynucleotide, nucleotide sequence, primer, oligonucleotide or probe, means natural or modified purine- and pyrimidine-containing polymers of any length, either polyribonucleotides or polydeoxyribonucleotides or mixed polyribo-polydeoxyribo nucleotides and α-anomeric forms thereof. The two or more purine- and pyrimidine-containing polymers are typically linked by a phosphoester bond or analog thereof. The terms can be used interchangeably to refer to all forms of nucleic acid, including deoxyribonucleic acid (DNA) and ribonucleic acid (RNA). The nucleic acids can be single strand, double, or triplex, linear or circular. Nucleic acids include genomic DNA, cDNA, and antisense. RNA nucleic acid can be spliced or unspliced mRNA, rRNA, tRNA or antisense. Nucleic acids include naturally occurring, synthetic, as well as nucleotide analogues and derivatives.

As a result of the degeneracy of the genetic code, nucleic acids include sequences degenerate with respect to sequences encoding fusion constructs of the invention. Thus, degenerate nucleic acid sequences encoding nucleolin binding peptides and fusion constructs of the invention are provided.

Nucleic acid can be produced using any of a variety of known standard cloning and chemical synthesis methods, and can be altered intentionally by site-directed mutagenesis or other recombinant techniques known to one skilled in the art. Purity of polynucleotides can be determined through sequencing, gel electrophoresis, UV spectrometry.

Nucleic acids may be inserted into a nucleic acid construct in which expression of the nucleic acid is influenced or regulated by an “expression control element,” referred to herein as an “expression cassette.” The term “expression control element” refers to one or more nucleic acid sequence elements that regulate or influence expression of a nucleic acid sequence to which it is operatively linked. An expression control element can include, as appropriate, promoters, enhancers, transcription terminators, gene silencers, a start codon (e.g., ATG) in front of a protein-encoding gene, etc.

An expression control element operatively linked to a nucleic acid sequence controls transcription and, as appropriate, translation of the nucleic acid sequence. The term “operatively linked” refers to a juxtaposition wherein the referenced components are in a relationship permitting them to function in their intended manner. Typically expression control elements are juxtaposed at the 5′ or the 3′ ends of the genes but can also be intronic.

Expression control elements include elements that activate transcription constitutively, that are inducible (i.e., require an external signal for activation), or derepressible (i.e., require a signal to turn transcription off; when the signal is no longer present, transcription is activated or “derepressed”). Also included in the expression cassettes of the invention are control elements sufficient to render gene expression controllable for specific cell-types or tissues (i.e., tissue-specific control elements). Typically, such elements are located upstream or downstream (i.e., 5′ and 3′) of the coding sequence. Promoters are generally positioned 5′ of the coding sequence. Promoters, produced by recombinant DNA or synthetic techniques, can be used to provide for transcription of the polynucleotides of the invention. A “promoter” is meant a minimal sequence element sufficient to direct transcription.

Nucleic acids may be inserted into a plasmid for propagation into a host cell and for subsequent genetic manipulation if desired. A plasmid is a nucleic acid that can be stably propagated in a host cell; plasmids may optionally contain expression control elements in order to drive expression of the nucleic acid. A vector is used herein synonymously with a plasmid and may also include an expression control element for expression in a host cell. Plasmids and vectors generally contain at least an origin of replication for propagation in a cell and a promoter. Plasmids and vectors are therefore useful for genetic manipulation of nucleolin binding peptide and fusion construct encoding nucleic acids, producing nucleolin binding peptides and fusion constructs or antisense nucleic acid, and expressing nucleolin binding peptides and fusion constructs in host cells and organisms, for example.

Bacterial system promoters include T7 and inducible promoters such as pL of bacteriophage λ, plac, ptrp, ptac (ptrp-lac hybrid promoter) and tetracycline responsive promoters. Trisect cell system promoters include constitutive or inducible promoters (e.g., ecdysone). Mammalian cell constitutive promoters include SV40, RSV, bovine papilloma virus (BPV) and other virus promoters, or inducible promoters derived from the genome of mammalian cells (e.g., metallothionein IIA promoter; heat shock promoter) or from mammalian viruses (e.g., the adenovirus late promoter; the inducible mouse mammary tumor virus long terminal repeat). Alternatively, a retroviral genome can be genetically modified for introducing and directing expression of nucleolin binding peptides and fusion constructs in appropriate host cells.

Expression systems further include vectors designed for in vivo use. Particular non-limiting examples include adenoviral vectors (U.S. Pat. Nos. 5,700,470 and 5,731,172), adeno-associated vectors (U.S. Pat. No. 5,604,090), herpes simplex virus vectors (U.S. Pat. No. 5,501,979), retroviral vectors (U.S. Pat. Nos. 5,624,820, 5,693,508 and 5,674,703), BPV vectors (U.S. Pat. No. 5,719,054) and CMV vectors (U.S. Pat. No. 5,561,063).

Yeast vectors include constitutive and inducible promoters (see, e.g., Ausubel et al., In: Current Protocols in Molecular Biology, Vol. 2, Ch. 13 (1988), eds., Greene Publish. Assoc. & Wiley Interscience; Grant et al. Methods in Enzymology, 153:516 (1987), eds., Wu & Grossman; Bitter Methods in Enzymology, 152:673 (1987), eds., Berger & Kimmel, Acad. Press, N.Y.; and, Strathern et al., The Molecular Biology of the Yeast Saccharomyces (1982), eds., Cold Spring Harbor Press, Vols. I and II). A constitutive yeast promoter such as ADH or LEU2 or an inducible promoter such as GAL may be used (R. Rothstein In: DNA Cloning, A Practical Approach, Vol. 11, Ch. 3 (1986), eds., D. M. Glover, IRL Press, Wash., D.C.). Vectors that facilitate integration of foreign nucleic acid sequences into a yeast chromosome, via homologous recombination for example, are known in the art. Yeast artificial chromosomes (YAC) are typically used when the inserted polynucleotides are too large for more conventional vectors (e.g., greater than about 12 Kb).

Expression vectors also can contain a selectable marker conferring resistance to a selective pressure or identifiable marker (e.g., beta-galactosidase), thereby allowing cells having the vector to be selected for, grown and expanded. Alternatively, a selectable marker can be on a second vector that is cotransfected into a host cell with a first vector containing a nucleic acid encoding a nucleolin binding peptide or fusion construct of the invention.

Selection systems include but are not limited to herpes simplex virus thymidine kinase gene (Wigler et al., Cell 11:223 (1977)), hypoxanthine-guanine phosphoribosyltransferase gene (Szybalska et al., Proc. Natl. Acad. Sci. USA 48:2026 (1962)), and adenine phosphoribosyltransferase (Lowy et al., Cell 22:817 (1980)) genes which can be employed in tk-, hgprt- or aprt-cells, respectively. Additionally, antimetabolite resistance can be used as the basis of selection for dhfr, which confers resistance to methotrexate (O'Hare et al., Proc. Natl. Acad. Sci. USA 78:1527 (1981)); the gpt gene, which confers resistance to mycophenolic acid (Mulligan et al., Proc. Natl. Acad. Sci. USA 78:2072 (1981)); neomycin gene, which confers resistance to aminoglycoside G-418 (Colberre-Garapin et al., J. Mol. Biol. 150:1(1981)); puromycin; and hygromycin gene, which confers resistance to hygromycin (Santerre et al., Gene 30:147 (1984)). Additional selectable genes include trpB, which allows cells to utilize indole in place of tryptophan; hisD, which allows cells to utilize histinol in place of histidine (Hartman et al., Proc. Natl. Acad. Sci. USA 85:8047 (1988)); and ODC (ornithine decarboxylase), which confers resistance to the ornithine decarboxylase inhibitor, 2-(difluoromethyl)-DL-ornithine, DFMO (McConlogue in: Current Communications in Molecular Biology (1987), Cold Spring Harbor Laboratory).

Host cells that express nucleolin binding peptides and fusion constructs, and host cells transformed with nucleic acids encoding nucleolin binding peptides and fusion constructs and vectors including a nucleic acid that encodes nucleolin binding peptide or fusion construct are also provided. In one embodiment, a host cell is a prokaryotic cell. In another embodiment, a host cell is a eukaryotic cell. In various aspects, the eukaryotic cell is a yeast or mammalian (e.g., human, primate, etc.) cell.

As used herein, a “host cell” is a cell into which a nucleic acid is introduced that can be propagated, transcribed, or encoded fusion construct expressed. The term also includes any progeny or subclones of the host cell. Host cells include cells that express a nucleolin binding peptide or fusion construct and cells that do not express a nucleolin binding peptides or fusion construct. Host cells that do not express a nucleolin binding peptide or fusion construct can be used to propagate nucleic acid or vector which includes a nucleic acid encoding a nucleolin binding peptide, a fusion construct, or an antisense.

Host cells include but are not limited to microorganisms such as bacteria and yeast; and plant, insect and mammalian cells. For example, bacteria transformed with recombinant bacteriophage nucleic acid, plasmid nucleic acid or cosmid nucleic acid expression vectors; yeast transformed with recombinant yeast expression vectors; plant cell systems infected with recombinant virus expression vectors (e.g., cauliflower mosaic virus, CaMV; tobacco mosaic virus, TMV) or transformed with recombinant plasmid expression vectors (e.g., Ti plasmid); insect cell systems infected with recombinant virus expression vectors (e.g., baculovirus); and animal cell systems infected with recombinant virus expression vectors (e.g., retroviruses, adenovirus, vaccinia virus), or transformed animal cell systems engineered for transient or stable propagation or expression.

Nucleolin binding peptides and fusion constructs, nucleic acids encoding nucleolin binding peptides and fusion constructs, vectors and host cells expressing nucleolin binding peptides and fusion constructs or transformed with nucleic acids encoding nucleolin binding peptides and fusion constructs or antisense include isolated and purified forms. The term “isolated,” when used as a modifier of an invention composition, means that the composition is made by the hand of man or is separated, substantially completely or at least in part, from the naturally occurring in vivo environment. Generally, an isolated composition is substantially free of one or more materials with which it normally associates with in nature, for example, one or more protein, nucleic acid, lipid, carbohydrate, cell membrane. The term “isolated” does not exclude alternative physical forms of the composition, such as multimers/oligomers, variants, modifications or derivatized forms, or forms produced synthetically or expressed in host cells produced by the hand of man. The term “isolated” also does not exclude forms (e.g., pharmaceutical formulations and combination compositions) in which there are combinations therein, any one of which is produced by the hand of man, and as such combinations do not typically exist in nature.

An “isolated” composition can also be “purified” when free of some, a substantial number of, most or all of the materials with which it typically associates with in nature. Thus, an isolated nucleolin binding peptide or fusion construct that also is substantially pure does not include polypeptides or polynucleotides present among millions of other sequences, such as proteins of a protein library or nucleic acids in a genomic or cDNA library, for example. A “purified” composition that includes a nucleolin binding peptide or fusion construct can be combined with one or more other molecules.

In accordance with the invention, there are provided nucleolin binding peptide and fusion construct compositions and combination compositions. In one embodiment, a composition includes one or more nucleolin binding peptides or fusion constructs and a pharmaceutically acceptable carrier or excipient. In another embodiment, a composition includes one or more nucleolin binding peptides or fusion constructs and an anti-cell proliferative, anti-tumor, anti-cancer, anti-neoplastic, anti-angiogenic or anti-inflammatory treatment or agent. In a further embodiment, a composition includes one or more nucleolin binding peptides or fusion constructs and an immune enhancing agent. Combinations, such as one or more nucleolin binding peptides or fusion constructs in a pharmaceutically acceptable carrier or excipient, with one or more of an anti-cell proliferative, anti-tumor, anti-cancer, anti-neoplastic, anti-angiogenic or anti-inflammatory treatment or agent, and an immune enhancing treatment or agent, are also provided.

Nucleolin binding peptides and fusion constructs of the invention can be used to target cells for lysis, cell death or apoptosis. Such cells can be selectively targeted. For example, a cell that expresses nucleolion can be targeted by a nucleolin binding peptide or fusion construct and thereby be preferentially killed compared to cells that express less nucleolin.

In accordance with the invention, there are provided methods of reducing or inhibiting proliferation of a cell, and methods of reducing or inhibiting cell proliferation. In one embodiment, a method includes contacting a cell with a nucleolin binding peptide or fusion construct in an amount sufficient to reduce or inhibit proliferation of the cell. In another embodiment, a method includes contacting a cell with a nucleolin binding peptide or fusion construct in an amount sufficient to reduce or inhibit cell proliferation.

Also provided are methods of reducing or inhibiting proliferation of a hyperproliferative cell, and methods of reducing or inhibiting proliferation of hyperproliferating cells. In one embodiment, a method includes contacting a hyperproliferative cell or hyperproliferating cells with a nucleolin binding peptide or fusion construct in an amount sufficient to reduce or inhibit proliferation.

Further provided are methods of reducing or inhibiting proliferation of a non-metastatic or metastatic neoplastic, cancer, tumor and malignant cell. In one embodiment, a method includes contacting a neoplastic, cancer, tumor or malignant cell with a nucleolin binding peptide or fusion construct in an amount sufficient to reduce or inhibit proliferation of the cell.

Still further provided are methods of reducing or inhibiting proliferation of a dormant or non-dividing non-metastatic or metastatic neoplastic, cancer, tumor and malignant cell. In one embodiment, a method includes contacting a dormant or non-dividing neoplastic, cancer, tumor or malignant cell with a nucleolin binding peptide or fusion construct in an amount sufficient to reduce or inhibit proliferation of the dormant or non-dividing cell.

Additionally provided are methods of selectively reducing or inhibiting proliferation of a cell (e.g., a hyperproliferating cell) that expresses nucleolin. In one embodiment, a method includes contacting the cell with a nucleolin binding peptide or fusion construct in an amount sufficient to reduce or inhibit proliferation of the cell (e.g., hyperproliferating cell), wherein the nucleolin binding moiety of said peptide binds to the nucleolin expressed by the cell.

Yet additionally provided are methods of selectively reducing or inhibiting proliferation of a neoplastic, tumor, cancer or malignant cell that expresses nucleolin. In one embodiment, a method includes contacting the cell with a nucleolin binding peptide or fusion construct in an amount sufficient to reduce or inhibit proliferation of the neoplastic, tumor, cancer or malignant cell, wherein the nucleolin binding moiety of the nucleolin binding peptide or fusion construct binds to the nucleolin expressed by the cell.

The term “contacting” means direct or indirect binding or interaction between two or more entities (e.g., between a nucleolin binding peptide or fusion construct and nucleolin or a nucleolin expressing cell). Contacting as used herein includes in solution, in solid phase, in vitro, ex vivo, in a cell and in vivo. Contacting in vivo can be referred to as administering, or administration, or in vivo delivery.

Cells to target for cytotoxicity, for example, in order to reduce, decrease or inhibit cell proliferation or survival, non-selectively or selectively, in vivo, in vitro and ex vivo, include cells that express nucleolin. Such cells include hyperproliferative cells, or cells undergoing undesirable proliferation or cell survival, as nucleolin is characterized as being expressed on a hyperproliferative cell, such as a neoplastic, cancer or tumor cell. Exemplary cells include benign and malignant hyperplasias, and non-metastatic and metastatic neoplasias, cancers, tumors and malignancies. Non-limiting examples of neoplastic, cancer and tumor cells in which nucleolin has been detected include, but are not limited to breast, lung, brain, lymphoid, gastrointestinal (mouth, esophagus, stomach, duodenum, ileum, jejunum (small intestine), colon, rectum), genito-urinary tract (uterus, ovary, vagina cervix, endometrium, fallopian tube, bladder, testicle, penis, prostate), kidney, pancreas, liver, bone marrow, lymph, blood, or skin.

Exemplary cells also include cells that are associated with or participate in angiogenesis, or cell migration or capillary tubule formation, such as endothelial cells. Endothelial cells are typically associated with all neoplasias, cancers, tumors and malignancies due to neovascularization of the neoplastic, cancer, tumor and malignant areas. Targeting endothelial cells can in turn result in cell cytotoxicity of such cells and vasculature, for example, thereby resulting in decreased blood supply, oxygen, energy and nutrient supplied to neoplasias, cancers, tumors and malignancies thereby resulting in cytotoxicity towards neoplasias, cancers, tumors and malignancies, even if such neoplasias, cancers, tumors and malignancies themselves do not express nucleolin.

Thus, nucleolin binding peptides, fusion constructs and the methods of the invention are applicable to treating undesirable or aberrant cell proliferation and hyperproliferative disorders, wherein the cells of such disorders do or do not express nucleolin. In accordance with the invention, methods of treating undesirable or aberrant cell proliferation and hyperproliferative disorders and diseases are provided. In one embodiment, a method includes administering to a subject (in need of treatment) an amount of a nucleolin binding peptide or fusion construct sufficient to treat the undesirable or aberrant cell proliferation or the hyperproliferative disorder or disease.

The term “hyperproliferative disorder” or “disease” refers to any undesirable or aberrant cell survival (e.g., failure to undergo programmed cell death or apoptosis), growth or proliferation. Such disorders include benign hyperplasias, non-metastatic and metastatic neoplasias, cancers, tumors and malignancies. Undesirable or aberrant cell proliferation and hyperproliferative disorders can affect any cell, tissue, organ in a subject. Undesirable or aberrant cell proliferation and hyperproliferative disorders can be present in a subject, locally, regionally or systemically. A hyperproliferative disorder can arise from a multitude of tissues and organs, including but not limited to breast, lung (e.g., small cell or non-small cell), thyroid, head and neck, brain, nasopharynx, throat, nose or sinuses, lymphoid, adrenal gland, pituitary gland, thyroid, lymph, gastrointestinal (mouth, esophagus, stomach, duodenum, ileum, jejunum (small intestine), colon, rectum), genito-urinary tract (uterus, ovary, vagina cervix, endometrium, fallopian tube, bladder, testicle, penis, prostate), kidney, pancreas, liver, bone, bone marrow, lymph, blood, muscle, skin, and stem cells, which may or may not metastasize to other secondary sites, regions or locations.

Nucleolin binding peptides, fusion constructs and methods of the invention are also applicable to metastatic or non-metastatic tumor, cancer, malignancy or neoplasia of any cell, organ or tissue origin. Such disorders can affect virtually any cell or tissue type, e.g., carcinoma, sarcoma, melanoma, neural, and reticuloendothelial or haematopoietic neoplastic disorders (e.g., myeloma, lymphoma or leukemia).

As used herein, the terms “neoplasia” and “tumor” refer to a cell or population of cells whose growth, proliferation or survival is greater than growth, proliferation or survival of a normal counterpart cell, e.g. a cell proliferative or differentiative disorder. A tumor is a neoplasia that has formed a distinct mass or growth. A “cancer” or “malignancy” refers to a neoplasia or tumor that can invade adjacent spaces, tissues or organs. A “metastasis” refers to a neoplasia, tumor, cancer or malignancy that has disseminated or spread from its primary site to one or more secondary sites, locations or regions within the subject, in which the sites, locations or regions are distinct from the primary tumor or cancer.

Neoplastic, tumor, cancer and malignant cells (metastatic or non-metastatic) include dormant or residual neoplastic, tumor, cancer and malignant cells. Such cells typically consist of remnant tumor cells that are not dividing (G0-G1 arrest). These cells can persist in a primary site or as disseminated neoplastic, tumor, cancer or malignant cells as a minimal residual disease. These dormant neoplastic, tumor, cancer or malignant cells remain unsymptomatic, but can develop severe symptoms and death once these dormant cells proliferate. Invention nucleolin binding peptides, fusion constructs and methods can be used to reduce or inhibit proliferation of dormant neoplastic, tumor, cancer or malignant cells, which can in turn inhibit or reduce tumor or cancer relapse, or tumor or cancer metastasis or progression.

In accordance with the invention, methods of treating a subject having a metastatic or non-metastatic tumor, cancer, malignancy or neoplasia are provided. In one embodiment, a method includes administering to a subject (in need of treatment) an amount of a nucleolin binding peptide or fusion construct sufficient to treat (e.g., reduce or inhibit proliferation) the metastatic or non-metastatic tumor, cancer, malignancy or neoplasia.

The metastatic or non-metastatic tumor, cancer, malignancy or neoplasia may be in any stage, e.g., early or advanced, such as a stage I, II, III, IV or V tumor. The metastatic or non-metastatic tumor, cancer, malignancy or neoplasia may have been subject to a prior treatment or be stabilized (non-progressing) or in remission.

In terms of metastasis, invention nucleolin binding peptides, fusion constructs and methods can be used to reduce or inhibit metastasis of a primary tumor or cancer to other sites, or the formation or establishment of metastatic tumors or cancers at other sites distal from the primary tumor or cancer thereby inhibiting or reducing tumor or cancer relapse or tumor or cancer progression. Thus, methods of the invention include, among other things, 1) reducing or inhibiting growth, proliferation, mobility or invasiveness of tumor or cancer cells that potentially or do develop metastases (e.g., disseminated tumor cells, DTC); 2) reducing or inhibiting formation or establishment of metastases arising from a primary tumor or cancer to one or more other sites, locations or regions distinct from the primary tumor or cancer; 3) reducing or inhibiting growth or proliferation of a metastasis at one or more other sites, locations or regions distinct from the primary tumor or cancer after a metastasis has formed or has been established; and 4) reducing or inhibiting formation or establishment of additional metastasis after the metastasis has been formed or established.

Cells of a benign or malignant, metastatic or non-metastatic tumor, cancer, malignancy or neoplasia may be aggregated in a “solid” cell mass or be dispersed or diffused. A “solid” tumor refers to cancer, neoplasia or metastasis that typically aggregates together and forms a mass. Specific non-limiting examples include visceral tumors such as melanomas, breast, pancreatic, uterine and ovarian cancers, testicular cancer, including seminomas, gastric or colon cancer, hepatomas, adrenal, renal and bladder carcinomas, lung, head and neck cancers and brain tumors/cancers.

Carcinomas, which refer to malignancies of epithelial or endocrine tissue, include respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from the uterus, cervix, lung, prostate, breast, head and neck, colon, pancreas, testes, adrenal, kidney, esophagus, stomach, liver and ovary. The term also includes carcinosarcomas, e.g., which include malignant tumors composed of carcinomatous and sarcomatous tissues. Adenocarcinoma includes a carcinoma of a glandular tissue, or in which the tumor forms a gland like structure.

Sarcomas refer to malignant tumors of mesenchymal cell origin. Exemplary sarcomas include for example, lymphosarcoma, liposarcoma, osteosarcoma, chondrosarcoma, leiomyosarcoma, rhabdomyosarcoma and fibrosarcoma.

Neural neoplasias include glioma, glioblastoma, meningioma, neuroblastoma, retinoblastoma, astrocytoma and oligodendrocytoma.

A “liquid tumor,” which refers to neoplasia that is dispersed or is diffuse in nature, as they do not typically form a solid mass. Particular examples include neoplasia of the reticuloendothelial or hematopoietic system, such as lymphomas, myelomas and leukemias. Non-limiting examples of leukemias include acute and chronic lymphoblastic, myeolblastic and multiple myeloma. Typically, such diseases arise from poorly differentiated acute leukemias, e.g., erythroblastic leukemia and acute megakaryoblastic leukemia. Specific myeloid disorders include, but are not limited to, acute promyeloid leukemia (APML), acute myelogenous leukemia (AML) and chronic myelogenous leukemia (CML). Lymphoid malignancies include, but are not limited to, acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Specific malignant lymphomas include, non-Hodgkin lymphoma and variants, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

As disclosed herein, undesirable or aberrant cell proliferation or hyperproliferative disorders can occur in uterus, breast, vagina, cervix and fallopian tube. Endometriosis occurs when cells of the uterus grow outside of the uterus and into other areas, such as ovaries, bladder or bowel. Fibroids and polyps can affect uterus, breast, vagina, cervix and fallopian tube.

Thus, in accordance with the invention, there are provided methods of treating benign hyperplasias, non-metastatic and metastatic neoplasias, cancers, tumors and malignancies in in uterus, breast, vagina, cervix, fallopian tube, ovary, bladder or bowel. In various embodiments, a method includes administering to a subject an amount of a nucleolin binding peptide or fusion construct sufficient to treat a benign hyperplasia, non-metastatic or metastatic neoplasia, cancer, tumor or malignancy in uterus, breast, vagina, cervix, fallopian tube, ovary, bladder or bowel.

As also disclosed herein, undesirable or aberrant cell proliferation or hyperproliferative disorders occur in prostate. Thus, in accordance with the invention, there are provided methods of treating benign prostate hyperplasia or metastatic prostate neoplasia. In one embodiment, a method includes administering to a subject an amount of a nucleolin binding peptide or fusion construct sufficient to treat benign prostate hyperplasia or metastatic prostate neoplasia.

Target cells also include cells that are associated with, participate in or are required for angiogenesis. Thus, in accordance with the invention, there are provided methods of decreasing, reducing or inhibiting angiogenesis. In one embodiment, a method includes administering to a subject an amount of a nucleolin binding peptide or fusion construct sufficient to decrease, reduce or inhibit angiogenesis in the subject.

Target cells further include cells that are associated with, participate in or are required for migration of endothelial cells or capillary-tubule formation. Thus, in accordance with the invention, there are provided methods of decreasing, reducing or inhibiting migration of endothelial cells or capillary-tubule formation. In one embodiment, a method includes administering to a subject an amount of a nucleolin binding peptide or fusion construct sufficient to decrease, reduce or inhibit migration of endothelial cells or capillary-tubule formation in the subject.

Target cells additionally include cells that are associated with, participate in or mediate an angiogenesis related or dependent disease or disorder. Thus, in accordance with the invention, there are provided methods of treating angiogenesis related or dependent diseases and disorders. In one embodiment, a method includes administering to a subject an amount of a nucleolin binding peptide or fusion construct sufficient to treat the angiogenesis related or dependent disease or disorder in the subject. Non-limiting exemplary angiogenesis related or dependent diseases and disorders include vascular tumors (angiofibromas, hemangiomatosis, hemangiosarcomas), cancers, neoplasias and malformations (arteriovenous malformations); reproductive disorders (endometriosis, placental insufficiency, pre-eclampsia etc); cardiovascular (atherosclerosis, coronary heart disease, vascular adhesions, vascular dementia, restenosis/re-perfusion injury), pulmonary (pulmonary fibrosis) or central nervous system diseases (Alzheimer's disease, cerebral autosomally dominant arteriopathy with subcortical infarcts and leucoencephalopathy (CADASIL), dementia etc.); syndromes such as Marfucci's syndrome, Rendu-Osler-Weber syndrome, von Hippel-Lindau syndrome; Hereditary hemorrhagic telangiectasia; ocular disorders such as macular degeneration, diabetic retinopathy, neovascular glaucoma, corneal graft neovascularization, ischemic retinopathy, retrolental fibroplasias, trachoma etc; a chronic or acute inflammatory disease such as rheumatoid arthritis, osteoarthritis, Crohn's disease, psoriasis, periodontitis, granulations-burns etc; aberrant wound repair such as hypertrophic scars, non-union fractures, pyrogenic granuloma etc; and metabolic disorders such as diabetes or obesity.

Any composition, treatment, protocol, therapy or regimen having an anti-cell proliferative activity or effect can be combined with a nucleolin binding peptide or fusion construct or used in combination in a method of the invention. Nucleolin binding peptides, fusion constructs and methods of the invention therefore include anti-proliferative, anti-tumor, anti-cancer, anti-neoplastic and anti-metastatic treatments, protocols and therapies, which include any other composition, treatment, protocol or therapeutic regimen that inhibits, decreases, retards, slows, reduces or prevents a hyperproliferative disorder, such as tumor, cancer, malignant or neoplastic growth, progression, metastasis, proliferation or survival, or worsening in vitro or in vivo. Particular non-limiting examples of an anti-proliferative (e.g., tumor) therapy include chemotherapy, immunotherapy, radiotherapy (ionizing or chemical), local thermal (hyperthermia) therapy, surgical resection and vaccination. A nucleolin binding peptide or fusion construct can be administered prior to, substantially contemporaneously with or following administration of the anti-cell proliferative, anti-neoplastic, anti-tumor, anti-cancer, anti-metastatic or immune-enhancing treatment or therapy. A nucleolin binding peptide or fusion construct can be administered as combination compositions with the anti-cell proliferative, anti-neoplastic, anti-tumor, anti-cancer, anti-metastatic or immune-enhancing treatment or therapy, metastatic or non-metastatic tumor, cancer, malignancy or neoplasia.

Anti-proliferative, anti-neoplastic, anti-tumor, anti-cancer and anti-metastatic compositions, methods, therapies, protocols or treatments include those that prevent, disrupt, interrupt, inhibit or delay cell cycle progression or cell proliferation; stimulate or enhance apoptosis or cell death, inhibit nucleic acid or protein synthesis or metabolism, inhibit cell division, or decrease, reduce or inhibit cell survival, or production or utilization of a necessary cell survival factor, growth factor or signaling pathway (extracellular or intracellular). Non-limiting examples of chemical agent classes having anti-cell proliferative, anti-neoplastic, anti-tumor, anti-cancer and anti-metastatic activities include alkylating agents, anti-metabolites, plant extracts, plant alkaloids, nitrosoureas, hormones, nucleoside and nucleotide analogues. Specific examples of drugs having anti-cell proliferative, anti-neoplastic, anti-tumor, anti-cancer and anti-metastatic activities include cyclophosphamide, azathioprine, cyclosporin A, prednisolone, melphalan, chlorambucil, mechlorethamine, busulphan, methotrexate, 6-mercaptopurine, thioguanine, 5-fluorouracil, cytosine arabinoside, AZT, 5-azacytidine (5-AZC) and 5-azacytidine related compounds such as decitabine (5-aza-2′deoxycytidine), cytarabine, 1-beta-D-arabinofuranosyl-5-azacytosine and dihydro-5-azacytidine, bleomycin, actinomycin D, mithramycin, mitomycin C, carmustine, lomustine, semustine, streptozotocin, hydroxyurea, cisplatin, mitotane, procarbazine, dacarbazine, taxol, vinblastine, vincristine, doxorubicin, dibromomannitol, etc.

Additional agents that are applicable with fusion constructs and methods are known in the art and can be employed. For example, biologicals such as antibodies, cell growth factors, cell survival factors, cell differentiative factors, cytokines and chemokines can be administered. Non-limiting examples of monoclonal antibodies include rituximab (Rituxan®), trastuzumab (Herceptin®), bevacizumab (Avastin®), cetuximab (Erbitux®), alemtuzumab (Campath®), panitumumab (Vectibix®), ibritumomab tiuxetan (Zevalin®), tositumomab (Bexxar®) etc., which can be used in combination with, inter alia, a fusion construct in accordance with the invention. Other targeted drugs that are applicable for use with the fusion constructs are imatinib (Gleevec®), gefitinib (Iressa®), bortzomib (Velcade®), lapatinib (Tykerb®), sunitinib (Sutent®), sorafenib (Nexavar®), nilotinib (Tasigna®), Erlotinib hydrochloride (Tarceva®) etc.

etc. Non-limiting examples of cell growth factors, cell survival factors, cell differentiative factors, cytokines and chemokines include IL-2, IL-1α, IL-1β, IL-3, IL-6, IL-7, granulocyte-macrophage-colony stimulating factor (GMCSF), IFN-γ, IL-12, TNF-α, TNFβ, MIP-1β, RANTES, SDF-1, MCP-1, MCP-2, MCP-3, MCP-4, eotaxin, eotaxin-2, I-309/TCA3, ATAC, HCC-1, HCC-2, HCC-3, LARC/MIP-3α, PARC, TARC, CKβ, CKβ6, CKβ7, CKβ9, CKβ11, CKη12, C10, IL-8, GROα, GROβ, ENA-78, GCP-2, PBP/CTAPIIIβ-TG/NAP-2, Mig, PBSF/SDF-1 and lymphotactin.

Additional non-limiting examples include immune-enhancing treatments and therapies, which include cell based therapies. In particular, immune-enhancing treatments and therapies include administering lymphocytes, plasma cells, macrophages, dendritic cells, NK cells and B-cells.

Methods of treating a metastatic or non-metastatic tumor, cancer, malignancy or neoplasia, methods of treating a subject in need of treatment due to having or at risk of having a metastatic or non-metastatic tumor, cancer, malignancy or neoplasia, and methods of increasing effectiveness or improving an anti-proliferative, anti-tumor, anti-cancer, anti-neoplasia, anti-malignancy, anti-angiogenic or anti-inflammatory or therapy are provided. In respective embodiments, a method includes administering to a subject with or at risk of a metastatic or non-metastatic tumor, cancer, malignancy or neoplasia, an amount of a nucleolin binding peptide or fusion construct sufficient to treat the metastatic or non-metastatic tumor, cancer, malignancy or neoplasia; administering to the subject an amount of a fusion construct sufficient to treat the subject; and administering to a subject that is undergoing or has undergone metastatic or non-metastatic tumor, cancer, malignancy or neoplasia therapy, an amount of a nucleolin binding peptide or fusion construct sufficient to increase effectiveness of the anti-proliferative, anti-tumor, anti-cancer, anti-neoplasia or anti-malignancy therapy.

Methods of the invention may be practiced prior to (i.e. prophylaxis), concurrently with or after evidence of the presence of undesirable or aberrant cell proliferation or a hyperproliferative disorder, disease or condition begins (e.g., one or more symptoms). Administering a nucleolin binding peptide or fusion construct prior to, concurrently with or immediately following development of a symptom of undesirable or aberrant cell proliferation or a hyperproliferative disorder may decrease the occurrence, frequency, severity, progression, or duration of one or more symptoms of the undesirable or aberrant cell proliferation or a hyperproliferative disorder, disease or condition in the subject. In addition, administering a nucleolin binding peptide or fusion construct prior to, concurrently with or immediately following development of one or more symptoms of the undesirable or aberrant cell proliferation or a hyperproliferative disorder, disease or condition may inhibit, decrease or prevent the spread or dissemination of hyperproliferating cells (e.g., metastasis) to other sites, regions, tissues or organs in a subject, or establishment of hyperproliferating cells (e.g., metastasis) at other sites, regions, tissues or organs in a subject.

Nucleolin binding peptides, fusion constructs and the methods of the invention, such as treatment methods, can provide a detectable or measurable therapeutic benefit or improvement to a subject. A therapeutic benefit or improvement is any measurable or detectable, objective or subjective, transient, temporary, or longer-term benefit to the subject or improvement in the condition, disorder or disease, an adverse symptom, consequence or underlying cause, of any degree, in a tissue, organ, cell or cell population of the subject. Therapeutic benefits and improvements include, but are not limited to, reducing or decreasing occurrence, frequency, severity, progression, or duration of one or more symptoms or complications associated with a disorder, disease or condition, or an underlying cause or consequential effect of the disorder, disease or condition. Nucleolin binding peptides, fusion constructs and methods of the invention therefore include providing a therapeutic benefit or improvement to a subject.

In a method of the invention in which a therapeutic benefit or improvement is a desired outcome, a nucleolin binding peptide or fusion construct can be administered in a sufficient or effective amount to a subject in need thereof. An “amount sufficient” or “amount effective” refers to an amount that likely provides, in single or multiple doses, alone or in combination, with one or more other compositions (therapeutic agents such as a chemotherapeutic or immune stimulating drug), treatments, protocols, or therapeutic regimens agents, a detectable response of any duration of time (long or short term), a desired outcome in or a benefit to a subject of any measurable or detectable degree or for any duration of time (e.g., for hours, days, months, years, or cured). The doses or “sufficient amount” or “effective amount” for treatment (e.g., to provide a therapeutic benefit or improvement) typically are effective to ameliorate a disorder, disease or condition, or one, multiple or all adverse symptoms, consequences or complications of the disorder, disease or condition, to a measurable extent, although reducing or inhibiting a progression or worsening of the disorder, disease or condition or a symptom, is considered a satisfactory outcome.

The term “ameliorate” means a detectable objective or subjective improvement in a subject's condition. A detectable improvement includes a subjective or objective reduction in the occurrence, frequency, severity, progression, or duration of a symptom caused by or associated with a disorder, disease or condition, an improvement in an underlying cause or a consequence of the disorder, disease or condition, or a reversal of the disorder, disease or condition.

Treatment can therefore result in inhibiting, reducing or preventing a disorder, disease or condition, or an associated symptom or consequence, or underlying cause; inhibiting, reducing or preventing a progression or worsening of a disorder, disease, condition, symptom or consequence, or underlying cause; or further deterioration or occurrence of one or more additional symptoms of the disorder, disease condition, or symptom. Thus, a successful treatment outcome leads to a “therapeutic effect,” or “benefit” or inhibiting, reducing or preventing the occurrence, frequency, severity, progression, or duration of one or more symptoms or underlying causes or consequences of a condition, disorder, disease or symptom in the subject. Treatment methods affecting one or more underlying causes of the condition, disorder, disease or symptom are therefore considered to be beneficial. Stabilizing or inhibiting progression or worsening of a disorder or condition is also a successful treatment outcome.

A therapeutic benefit or improvement therefore need not be complete ablation of any one, most or all symptoms, complications, consequences or underlying causes associated with the condition, disorder or disease. Thus, a satisfactory endpoint is achieved when there is an incremental improvement in a subject's condition, or a partial reduction in the occurrence, frequency, severity, progression, or duration, or inhibition or reversal, of one or more associated adverse symptoms or complications or consequences or underlying causes, worsening or progression (e.g., stabilizing one or more symptoms or complications of the condition, disorder or disease), of one or more of the physiological, biochemical or cellular manifestations or characteristics of the disorder or disease, over a short or long duration of time (hours, days, weeks, months, etc.).

In particular embodiments, a method of treatment results in partial or complete destruction of a metastatic or non-metastatic tumor, cancer, malignant or neoplastic cell mass, volume, size or numbers of cells; results in stimulating, inducing or increasing metastatic or non-metastatic tumor, cancer, malignant or neoplastic cell necrosis, lysis or apoptosis; results in reducing metastatic or non-metastatic tumor, cancer, malignant or neoplastic volume, size, cell mass; results in inhibiting or preventing progression or an increase in metastatic or non-metastatic tumor, cancer, malignant or neoplastic volume, mass, size or cell numbers; results in inhibiting or decreasing the spread or dissemination of hyperproliferating cells (e.g., metastasis) to other (secondary) sites, regions, tissues or organs in a subject, or establishment of hyperproliferating cells (e.g., metastasis) at other (secondary) sites, regions, tissues or organs in a subject; reduces, inhibits or decreases angiogenesis or inflammation, or results in prolonging lifespan of the subject. In additional particular embodiments, a method of treatment results in reducing or decreasing severity, duration or frequency of an adverse symptom or complication associated with or caused by the metastatic or non-metastatic tumor, cancer, malignancy or neoplasia.

An amount sufficient or an amount effective can but need not be provided in a single administration and, can but need not be, administered alone or in combination with another composition (e.g., chemotherapeutic or immune enhancing or stimulating agent), treatment, protocol or therapeutic regimen. For example, the amount may be proportionally increased as indicated by the need of the subject, status of the disorder, disease or condition treated or the side effects of treatment. In addition, an amount sufficient or an amount effective need not be sufficient or effective if given in single or multiple doses without a second composition (e.g., chemotherapeutic or immune stimulating agent), treatment, protocol or therapeutic regimen, since additional doses, amounts or duration above and beyond such doses, or additional compositions (e.g., chemotherapeutic or immune stimulating agents), treatments, protocols or therapeutic regimens may be included in order to be considered effective or sufficient in a given subject. Amounts considered sufficient also include amounts that result in a reduction of the use of another treatment, therapeutic regimen or protocol.

An amount sufficient or an amount effective need not be effective in each and every subject treated, prophylactically or therapeutically, nor a majority of treated subjects in a given group or population. As is typical for treatment or therapeutic methods, some subjects will exhibit greater or less response to a given treatment, therapeutic regimen or protocol. An amount sufficient or an amount effective refers to sufficiency or effectiveness in a particular subject, not a group or the general population. Such amounts will depend in part upon the condition treated, such as the type or stage of undesirable or aberrant cell proliferation or hyperproliferative disorder (e.g., a metastatic or non-metastatic tumor, cancer, malignancy or neoplasia), angiogenesis, inflammation, the therapeutic effect desired, as well as the individual subject (e.g., the bioavailability within the subject, gender, age, etc.).

Particular non-limiting examples of therapeutic benefit or improvement for undesirable or aberrant cell proliferation, such as a hyperproliferative disorder (e.g., a metastatic or non-metastatic tumor, cancer, malignancy or neoplasia) include a reduction in cell size, mass or volume, endothelial cells, inhibiting an increase in cell size, mass or volume, a slowing or inhibition of worsening or progression, stimulating cell necrosis, lysis or apoptosis, reducing or inhibiting neoplastic or tumor malignancy or metastasis, reducing mortality, angiogenesis or inflammation, and prolonging lifespan of a subject. Thus, inhibiting or delaying an increase in cell size, mass, volume or metastasis (stabilization) can increase lifespan (reduce mortality) even if only for a few days, weeks or months, even though complete ablation of the metastatic or non-metastatic tumor, cancer, malignancy or neoplasia has not occurred. Adverse symptoms and complications associated with a hyperproliferative disorder (e.g., a metastatic or non-metastatic tumor, cancer, malignancy or neoplasia), angiogenesis or inflammation that can be reduced or decreased include, for example, pain, nausea, discomfort, lack of appetite, lethargy and weakness. A reduction in the occurrence, frequency, severity, progression, or duration of a symptom of undesirable or aberrant cell proliferation, such as a hyperproliferative disorder (e.g., a metastatic or non-metastatic tumor, cancer, malignancy or neoplasia), angiogenesis or inflammation, such as an improvement in subjective feeling (e.g., increased energy, appetite, reduced nausea, improved mobility or psychological well being, etc.), are therefore all examples of therapeutic benefit or improvement.

For example, a sufficient or effective amount of a nucleolin binding peptide or fusion construct is considered as having a therapeutic effect if administration results in less chemotherapeutic drug, radiation or immunotherapy being required for treatment of undesirable or aberrant cell proliferation, such as a hyperproliferative disorder (e.g., a metastatic or non-metastatic tumor, cancer, malignancy or neoplasia), angiogenesis or inflammation.

The term “subject” refers to animals, typically mammalian animals, such as humans, non human primates (apes, gibbons, chimpanzees, orangutans, macaques), domestic animals (dogs and cats), farm animals (horses, cows, goats, sheep, pigs) and experimental animal (mouse, rat, rabbit, guinea pig). Subjects include animal disease models, for example, animal models of undesirable or aberrant cell proliferation, such as a hyperproliferative disorder (e.g., a metastatic or non-metastatic tumor, cancer, malignancy or neoplasia), angiogenesis or inflammation for analysis of nucleolin binding peptides and fusion constructs in vivo.

Subjects appropriate for treatment include those having or at risk of having a metastatic or non-metastatic tumor, cancer, malignant or neoplastic cell, angiogenesis or inflammation, those undergoing as well as those who are undergoing or have undergone anti-proliferative (e.g., metastatic or non-metastatic tumor, cancer, malignancy or neoplasia), anti-angiogenesis or anti-inflammation therapy, including subjects where the tumor is in remission. “At risk” subjects typically have risk factors associated with undesirable or aberrant cell proliferation, development of hyperplasia (e.g., a tumor), angiogenesis or inflammation.

At risk subjects also include those that are candidates for and those that have undergone surgical resection, chemotherapy, immunotherapy, ionizing or chemical radiotherapy, local or regional thermal (hyperthermia) therapy, or treatment for angiogenesis or inflammation. The invention is therefore applicable to treating a subject who is at risk of a metastatic or non-metastatic tumor, cancer, malignancy, neoplasia angiogenesis or inflammation, or a complication associated with a metastatic or non-metastatic tumor, cancer, malignancy, neoplasia, angiogenesis or inflammation, for example, due to metastatic or non-metastatic tumor, cancer, malignancy or neoplasia reappearance or regrowth following a period of stability or remission.

Risk factors include gender, lifestyle (diet, smoking), occupation (medical and clinical personnel, agricultural and livestock workers), environmental factors (carcinogen exposure), family history (autoimmune disorders, diabetes, etc.), genetic predisposition, etc. For example, subjects at risk for developing melanoma include excess sun exposure (ultraviolet radiation), fair skin, high numbers of naevi (dysplastic nevus), patient phenotype, family history, or a history of a previous melanoma. Subjects at risk for developing cancer can therefore be identified by lifestyle, occupation, environmental factors, family history, and genetic screens for tumor associated genes, gene deletions or gene mutations. Subjects at risk for developing breast cancer lack Brcal, for example. Subjects at risk for developing colon cancer have early age or high frequency polyp formation, or deleted or mutated tumor suppressor genes, such as adenomatous polyposis coli (APC), for example.

Subjects also include those precluded from other treatments. For example, certain subjects may not be good candidates for surgical resection, chemotherapy, immunotherapy, ionizing or chemical radiotherapy, local or regional thermal (hyperthermia) therapy, or vaccination. Thus, candidate subjects for treatment in accordance with the invention include those that are not a candidate for surgical resection, chemotherapy, immunotherapy, ionizing or chemical radiotherapy, local or regional thermal (hyperthermia) therapy, or vaccination.

Nucleolin binding peptides and fusion constructs can be formulated in a unit dose or unit dosage form. In a particular embodiment, a nucleolin binding peptide or fusion construct is in an amount effective to treat a subject having undesirable or aberrant cell proliferation or a hyperproliferative disorder. In an additional particular embodiment, a nucleolin binding peptide or fusion construct is in an amount effective to treat a subject having a metastatic or non-metastatic tumor, cancer, malignancy, neoplasia, angiogenesis or inflammation. In a further particular embodiment, a nucleolin binding peptide or fusion construct is in an amount effective to reduce fertility of a subject. Exemplary unit doses range from about 25-250, 250-500, 500-1000, 1000-2500 or 2500-5000, 5000-25,000, 5000-50,000 ng; and from about 25-250, 250-500, 500-1000, 1000-2500 or 2500-5000, 5000-25,000, 5000-50,000 μg.

Compositions and methods of the invention may be contacted or provided in vitro, ex vivo or in vivo. Compositions can be administered to provide the intended effect as a single or multiple dosages, for example, in an effective or sufficient amount. Exemplary doses range from about 25-250, 250-500, 500-1000, 1000-2500 or 2500-5000, 5000-25,000, 5000-50,000 pg/kg; from about 50-500, 500-5000, 5000-25,000 or 25,000-50,000 ng/kg; and from about 25-250, 250-500, 500-1000, 1000-2500 or 2500-5000, 5000-25,000, 5000-50,000 μg/kg, on consecutive days, or alternating days or intermittently. Single or multiple doses can be administered on consecutive days, alternating days or intermittently.

Compositions can be administered and methods may be practiced via systemic, regional or local administration, by any route. For example, a nucleolin binding peptide or fusion construct can be administered systemically, regionally or locally, intravenously, orally (e.g., ingestion or inhalation), intramuscularly, intraperitoneally, intradermally, subcutaneously, intracavity, intracranially, transdermally (topical), parenterally, e.g. transmucosally or rectally. Compositions and methods of the invention including pharmaceutical formulations can be administered via a (micro)encapsulated delivery system or packaged into an implant for administration.

The invention further provides nucleolin binding peptides, fusion constructs and methods wherein the nucleolin binding peptides or fusion constructs are included in pharmaceutical compositions. A pharmaceutical composition refers to “pharmaceutically acceptable” and “physiologically acceptable” carriers, diluents or excipients. As used herein, the term “pharmaceutically acceptable” and “physiologically acceptable,” when referring to carriers, diluents or excipients includes solvents (aqueous or non-aqueous), detergents, solutions, emulsions, dispersion media, coatings, isotonic and absorption promoting or delaying agents, compatible with pharmaceutical administration and with the other components of the formulation. Such formulations can be contained in a tablet (coated or uncoated), capsule (hard or soft), microbead, emulsion, powder, granule, crystal, suspension, syrup or elixir.

Pharmaceutical compositions can be formulated to be compatible with a particular route of administration. Compositions for parenteral, intradermal, or subcutaneous administration can include a sterile diluent, such as water, saline solution, fixed oils, polyethylene glycols, glycerine, propylene glycol or other synthetic solvents. The preparation may contain one or more preservatives to prevent microorganism growth (e.g., antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose).

Pharmaceutical compositions for injection include sterile aqueous solutions (where water soluble) or dispersions and sterile powders for the extemporaneous preparation of sterile injectable solutions or dispersion. For intravenous administration, suitable carriers include physiological saline, bacteriostatic water, an organic solvent such as Cremophor EL™ (BASF, Parsippany, N.J.) or phosphate buffered saline (PBS). The carrier can be a solvent or dispersion medium containing, for example, water, ethanol, polyol (e.g., glycerol, propylene glycol, and polyetheylene glycol), polysorbate (Tween-20 or Tween-80) and suitable mixtures thereof. Fluidity can be maintained, for example, by the use of a coating such as lecithin, or by the use of surfactants. Antibacterial and antifungal agents include, for example, parabens, chlorobutanol, phenol, ascorbic acid and thimerosal. Including an agent that delays absorption, for example, aluminum monostearate and gelatin can prolonged absorption of injectable compositions.

Additional pharmaceutical formulations and delivery systems are known in the art and are applicable in the methods of the invention (see, e.g., Remington's Pharmaceutical Sciences (1990) 18th ed., Mack Publishing Co., Easton, Pa.; The Merck Index (1996) 12th ed., Merck Publishing Group, Whitehouse, N.J.; Pharmaceutical Principles of Solid Dosage Forms, Technonic Publishing Co., Inc., Lancaster, Pa., (1993); and Poznansky, et at., Drug Delivery Systems, R. L. Juliano, ed., Oxford, N.Y. (1980), pp. 253-315).

The invention provides kits including nucleolin binding peptides or fusion constructs of the invention, combination compositions and pharmaceutical formulations thereof, packaged into suitable packaging material. A kit optionally includes a label or packaging insert including a description of the components or instructions for use in vitro, in vivo, or ex vivo, of the components therein. Exemplary instructions include instructions for reducing or inhibiting proliferation of a cell, reducing or inhibiting proliferation of undesirable or aberrant cells, such as a hyperproliferating cell, reducing or inhibiting proliferation of a metastatic or non-metastatic tumor, cancer, malignant or neoplastic cell, treating a subject having a hyperproliferative disorder, treating a subject having a metastatic or non-metastatic tumor, cancer, malignancy or neoplasia, or reducing fertility of an animal.

A kit can contain a collection of such components, e.g., two or more fusion constructs alone, or in combination with another therapeutically useful composition (e.g., an anti-proliferative or immune-enhancing drug).

The term “packaging material” refers to a physical structure housing the components of the kit. The packaging material can maintain the components sterilely, and can be made of material commonly used for such purposes (e.g., paper, corrugated fiber, glass, plastic, foil, ampules, vials, tubes, etc.).

Kits of the invention can include labels or inserts. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a disk (e.g., hard disk), optical disk such as CD- or DVD-ROM/RAM, DVD, MP3, magnetic tape, or an electrical storage media such as RAM and ROM or hybrids of these such as magnetic/optical storage media, FLASH media or memory type cards.

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer information, lot numbers, manufacturer location and date.

Labels or inserts can include information on a condition, disorder, disease or symptom for which a kit component may be used. Labels or inserts can include instructions for the clinician or for a subject for using one or more of the kit components in a method, treatment protocol or therapeutic regimen. Instructions can include dosage amounts, frequency or duration, and instructions for practicing any of the methods, treatment protocols or therapeutic regimes set forth herein. Exemplary instructions include, instructions for treating an undesirable or aberrant cell proliferation, hyperproliferating cells and disorders (e.g., metastatic or non-metastatic tumor, cancer, malignancy or neoplasia). Kits of the invention therefore can additionally include labels or instructions for practicing any of the methods of the invention described herein including treatment methods.

Labels or inserts can include information on any benefit that a component may provide, such as a prophylactic or therapeutic benefit. Labels or inserts can include information on potential adverse side effects, such as warnings to the subject or clinician regarding situations where it would not be appropriate to use a particular composition. Adverse side effects could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Invention kits can additionally include other components. Each component of the kit can be enclosed within an individual container and all of the various containers can be within a single package. Invention kits can be designed for cold storage. Invention kits can further be designed to contain host cells expressing a nucleolin binding peptide or fusion construct of the invention, or that contain nucleic acids encoding a nucleolin binding peptide or fusion construct. The cells in the kit can be maintained under appropriate storage conditions until the cells are ready to be used. For example, a kit including one or more cells can contain appropriate cell storage medium so that the cells can be thawed and grown.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All patents, applications, publications, other references, GenBank citations and ATCC citations cited herein are expressly incorporated by reference herein in their entirety. In case of conflict, the specification, including definitions, will control.

As used herein, the singular forms “a”, “and,” and “the” include plural referents unless the context clearly indicates otherwise. Thus, for example, reference to “a nucleolin binding peptide or fusion construct” includes a plurality of such nucleolin binding peptides or fusion constructs, and so forth.

As used herein, all numerical values or numerical ranges include integers within such ranges and fractions of the values or the integers within ranges unless the context clearly indicates otherwise. Thus, for example, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. Reference to a range of 1-5,000 fold includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth. A reference to a range includes a reference to subranges within that range. Reference to a series of ranges, for example, reference to a range of 30-40%, 40-50%, 50-60%, 60-70%, 70-80%, or 90-100%, include combinations of combined ranges, such as 30-50%, 50-70%, 70-100%, etc. A series of ranges include both lower and upper ends of those ranges combined into ranges. Thus, for example, reference to a series of ranges such as 50-100 100-200, and 200-300, includes a range of 50-200, 50-300, 100-300, etc.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include aspects that are not expressly included in the invention are nevertheless disclosed herein.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

### Example 1

This example includes a description of designed peptides.

A computer simulation was used to design a 14-amino acid helical peptide from a nucleolin binding sequence of HMGN2-derived molecule (F3 peptide). F3 is a 31 amino acid protein with the sequence KDEPQRRSARLSAKPAPPKPEPKPKKAPAKK, which has been reported to bind to nucleolin (Porkka K et al., Proc. Natl. Acad. Sci. USA. 99:7444 (2002)). The first 14 amino acids of F3, KDEPQRRSARLSAK, show a short helical structure from residues 9 to 11. A substituted peptide sequence was made by replacing the KDEP sequence with RARL to RARLQRRSARLSAK and to increase helicity. Various length of the nucleolin binding analogues were synthesized with the primary sequence of: R₁ARLQRRSARL₁₁, R₁ARLQRRSARLS₁₂, H₁ARLQRRSARLSAK₁₄, R₁VRLQRRSARLSAK₁₄, and H₁AHLQRRSARLSAK₁₄.

A fusion construct was subsequently designed by conjugating the first 14 amino acids of F3 via a peptide bond at the C terminus of an 18 amino acid lytic peptide, KFAKFAKKFAKFAKKFAK, to form a 32 amino acid fusion construct K₁FAKFAKKFAKFAKKFAKKD₂₀EPQRRSARLSAK₃₂(subscripts refer to the position of amino acid in the sequence). This fusion construct is hereafter referred to as EP-301.

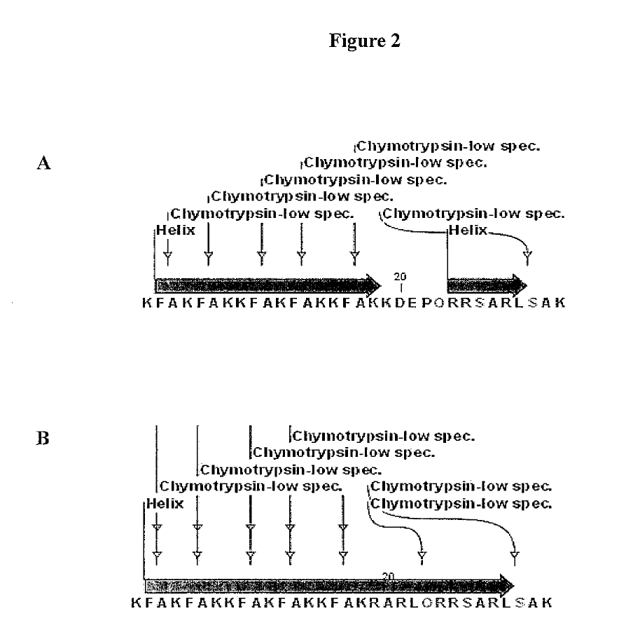

Analysis of the fusion construct showed absence of helicity from amino acids 19-22 (KDEP) of the molecule. Substitution of KDEP with RARL sequence resulted in a continuous alpha helical fusion construct from amino acids 1 to 29 of the fusion construct. The full sequence of this fusion construct is KFAKFAKKFAKFAKK₁FAKR₁₉ARLQRRSARLSAK₃₂, which is hereafter called EP-302. Computer representations of EP-301 and EP-302 are shown in FIG. 2.

### Example 2

This example includes studies of the anticancer activity of nucleolin binding peptides.

Nucleolin binding peptides were analyzed for anti-cancer activity in vitro against MDA-MB-435S breast cancer cells. KDEPQRRSARLSAKPAPPKPEPKPKKAPAKK (F3 peptide), KDEPQRRSARLSAK (F3-14 mer fragment), RARLQRRSARLSAK, R₁ARLQRRSARL₁₁, R₁ARLQRRSARLS₁₂, H₁ARLQRRSARLSAK₁₄, R₁VRLQRRSARLSAK₁₄, and H₁AHLQRRSARLSAK₁₄were freshly dissolved in saline and added into cancer cell seeded multi-well plates at increasing concentrations of 0, 0.001, 0.01, 0.1, 1, 2, 5, 10 and 100 μM. Incubations were conducted for 120 h at 37° C. Cell viability was determined using formazan conversion assays (MTT assays). Controls were treated with USP saline or 0.1% TritonX-100™ as reference for 0 and 100% cell death, respectively. Cytotoxicity was evaluated as an IC₅₀ values.

Results (Table 1) showed that the newly designed ligand had superior anti-cancer activity compared to F3 and the 14 mer F3 peptide. The highest activity was found for R1VRLQRRSARLSAK14 and R₁ARLQRRSARLSAK₁₄ that had high cell killing activity (IC₅₀=1.9-2.3 μM) compared to K₁DEPQRRSARLSAK₁₄PAPPKPEPKPKKAPAKK₃₁ at 17.3 μM or K₁DEPQRRSARLSAK₁₄11.1 μM.

TABLE 1
Anti-cancer activity of nucleolin
binding ligands.

| | IC₅₀ [μM] |
|---|---|
| K₁DEPQRRSARLSAK₁₄PAPPKPEPKPK | 17.31 ± 1.25 |
| KAPAKK₃₁ | |
| K₁DEPQRRSARLSAK₁₄ | 11.1 ± 0.3 |
| R₁ARLQRRSARLSAK₁₄ | 2.4 ± 0.4 |
| R₁ARLQRRSARL₁₁ | 5.4 ± 1.2 |
| R₁ARLQRRSARLS₁₂ | 6.5 ± 0.7 |
| H₁ARLQRRSARLSAK₁₄ | 3.2 ± 2.2 |
| R₁VRLQRRSARLSAK₁₄ | 1.9 ± 0.9 |
| H₁AHLQRRSARLSAK₁₄ | 4.7 ± 0.14 |

Nucleolin binding ligands kill cancer cells at low micromolar concentrations.

### Example 3

This example includes a description of designed fusion constructs.

A fusion construct was subsequently designed by conjugating the first 14 amino acids of F3 via a peptide bond at the C terminus of an18 amino acid lytic peptide, KFAKFAKKFAKFAKKFAK, to form a 32 amino acid fusion construct K₁FAKFAKKFAKFAKKFAKKD₂₀EPQRRSARLSAK₃₂(subscripts refer to the position of amino acid in the sequence). This fusion construct is hereafter referred to as EP-301.

Analysis of the fusion construct showed absence of helicity from amino acids 19-22 (KDEP) of the molecule. Substitution of KDEP with RARL sequence resulted in a continuous alpha helical fusion construct from amino acids 1 to 29 of the fusion construct. The full sequence of this fusion construct is K₁FAKFAKKFAKFAKKFAKR₁₉ARLQRRSARLSAK₃₂, which is hereafter called EP-302. Computer representations of EP-301 and EP-302 are shown in FIG. 2.

### Example 4

This example includes studies of the anticancer activity of peptide fusion constructs.

EP-301 and EP-302 were analyzed for anti-cancer activity in vitro in a time course experiment against MDA-MB-435S breast cancer cells. EP-301 and EP-302 were freshly dissolved in saline and added into cancer cell seeded multi-well plates at increasing concentrations of 0, 0.001, 0.01, 0.1, 1, 2, 5, 10 and 100 μM. Incubations were conducted for 0.25, 0.5, 1, 2, 4, 6 h or 24 h at 37° C. Cell viability was determined using formazan conversion assays (MTT assays). Controls were treated with USP saline or 0.1% TritonX-100™ as reference for 0 and 100% cell death, respectively. Cytotoxicity was evaluated as an IC₅₀ value derived for EP-301 and EP-302 at each time point.

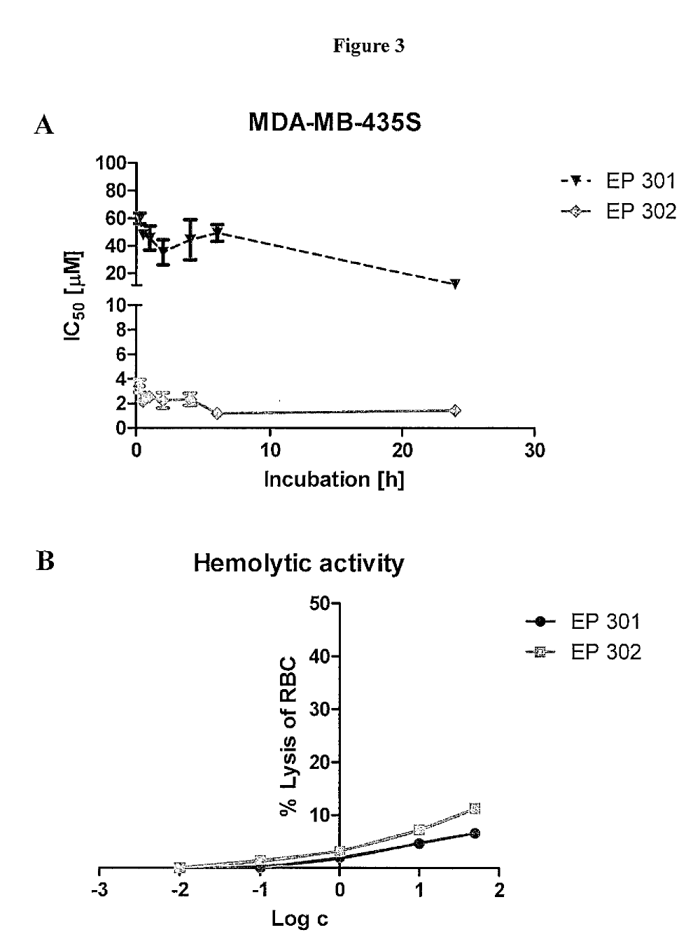

Results (FIG. 3) showed that EP-302 had high cell killing activity within 30 minutes (IC₅₀=2.3 μM) compared to EP-301, which was inactive (IC₅₀=48 μM). EP-301 shows activity of >40 μM after 6 h and reached maximal activity of 12 μM after 24 h.

### Example 5

This example includes studies of the anticancer activity and hemolytic activity of different peptide fusion constructs.

Twelve different fusion constructs were analyzed for anti-cancer activity in vitro against MDA-MB-435S breast cancer cells and for hemolytic activity in vitro to human red blood cells. Fusion constructs of the following sequence were tested:

K₁FAKFAKKFAKFAKKFAKKD₂₀EPQRRSARLSAK₃₂,
KFAKFAKKFAKFAKKFAKR₁₉ARLQRRSARLSAK₃₂,
KFAKFAKKFAKFAKKFAKV₁₉ARLQRRFARLFAK₃₂,
KFAKFAKKFAKFAKKFAKK₁₉RAPQRRSARLSAK₃₂,
KFAKFAKKFAKFAKKFAKV₁₉ARLQRRFA₂₇,
KFAKFAKKFAKFAKKFAKR₁₉ARLQRRF₂₆,
KFAKFAKKFAKFAKKFAKR₁₉ARLQRR₂₅,
KFAKFAKKFAKFAKKFAKR₁₉LQRRFARLFAK₃₀,
KFAKFAKKFAKFAKKFAKR₁₉LQRRSARLSAK₃₀,
KFAKFAKKFAKFAKKFAKF₁₉ARLFAK₂₅,
KFAKFAKKFAKFAKKFAKS₁₉ARLSAK₂₅,
KFAKFAKKFAKFAKKFAKR₁₉ARLQ₂₃

The peptide conjugates were freshly dissolved in saline and added into cancer cell seeded multi-well plates at increasing concentrations of 0, 0.001, 0.01, 0.1, 1, 2, 5, 10 and 100 μM. Incubations were conducted for 4 h at 37° C. Cell viability was determined using formazan conversion assays (MTT assays). Controls were treated with USP saline or 0.1% TritonX-100™ as reference for 0 and 100% cell death, respectively. Cytotoxicity was evaluated as an IC₅₀ value.

Hemolytic activity was studied by incubating the fusion constructs with human red blood cells at concentrations of 0.01, 0.1, 1, 10 and 50 μM for 2 hours. Saline and 0.1% Triton served as controls.

Results (Table 2) showed that eight out of 10 nucleolin targeting peptide conjugates had high cell killing activity in the low micromolar range (IC₅₀=2.3 μM) compared to EP-301 (K₁FAKFAKKFAKFAKKFAKKD₂₀EPQRRSARLSAK₃₂) or KFAKFAKKFAKFAKKFAKK₁₉RAPQRRSARLSAK₃₂, which had the lowest acitivty (IC₅₀=64.8 and 7.3 μM). The shortest peptide conjugates KFAKFAKKFAKFAKKFAKS₁₉ARLSAK₂₅ and KFAKFAKKFAKFAKKFAKR₁₉ARLQ₂₃ had lowest activities (IC₅₀ 4.1 and 3.6 μM).

Six out of 12 nucleolin targeting peptide conjugates were not hemolytic. KFAKFAKKFAKFAKKFAKV₁₉ARLQRRFARLFAK₃₂, KFAKFAKKFAKFAKKFAKR19LQRRFARLFAK₃₀ and KFAKFAKKFAKFAKKFAKR₁₉ARLQ₂₃ had hemolytic activities (HA₅₀) greater than 100 μM. Three fusion constructs had hemolytic activities (HA₅₀) below 100 μM: KFAKFAKKFAKFAKKFAKS₁₉ARLSAK₂₅ (69 μM), KFAKFAKKFAKFAKKFAKR₁₉LQRRSARLSAK₃₀ (56 μM), and KFAKFAKKFAKFAKKFAKF₁₉ARLFAK₂₅ (14.3 μM).

TABLE 2
In vitro activities of nucleolin targeting lytic peptide
conjugates to MDA-MB-435S breast cancer cells
and hemolytic activity (HA50) to human red blood cells.

| Nucleolin targeting lytic peptide conjugates | IC₅₀ [μM] | HA₅₀ [μM] |
|---|---|---|
| K₁FAKFAKKFAKFAKKFAKKD₂₀EPQRRSARLSAK₃₂ | 64.8 ± 14 | Not hemolytic |
| KFAKFAKKFAKFAKKFAKR₁₉ARLQRRSARLSAK₃₂ | 0.9 ± 0.05 | Not hemolytic |
| KFAKFAKKFAKFAKKFAKV₁₉ARLQRRFARLFAK₃₂ | 0.4 ± 0.002 | 205 ± 13.1 |
| KFAKFAKKFAKFAKKFAKK₁₉RAPQRRSARLSAK₃₂ | 7.3 ± 0.42 | Not hemolytic |
| KFAKFAKKFAKFAKKFAKV₁₉ARLQRRFA₂₇ | 0.8 ± 0.16 | Not hemolytic |
| KFAKFAKKFAKFAKKFAKR₁₉ARLQRRF₂₆ | 0.87 ± 0.003 | Not hemolytic |
| KFAKFAKKFAKFAKKFAKR₁₉ARLQRR₂₅ | 1.6 ± 0.05 | Not hemolytic |
| KFAKFAKKFAKFAKKFAKR₁₉LQRRFARLFAK₃₀ | 0.74 ± 0.12 | 123 ± 11.2 |
| KFAKFAKKFAKFAICKFAKR₁₉LQRRSARLSAK₃₀ | 2.2 ± 0.3 | 56 ± 8.5 |
| KFAKFAKKFAKFAKKFAKF₁₉ARLFAK₂₅ | 1.23 ± 0.7 | 14.3 ± 2.1 |
| KFAKFAKKFAKFAKKFAKS₁₉ARLSAK₂₅ | 4.1 ± 0.2 | 69 ± 6.9 |
| KFAKFAKKFAKFAKKFAKR₁₉ARLQ₂₃ | 3.6 ± 0.09 | 107 ± 8.5 |

### Example 6

This example includes cytotoxicity studies of a peptide construct in various cancer cell lines.

EP-302 was freshly dissolved in saline and added into cancer cell seeded multi-well plates at increasing concentrations of 0, 0.001, 0.01, 0.1, 1, 2, 5, 10 and 100 μM. Incubations were conducted for 6 h or 24 h at 37° C. Cell viability was determined using formazan conversion assays (MTT assays). Controls were treated with USP saline or 0.1% TritonX-100™ as reference for 0 and 100% cell death, respectively. Cytotoxicity was evaluated as an IC₅₀ value derived for EP-302 in each cell line.

As shown in Table 3, most of the cancer cell lines were highly sensitive to EP-302. Some colon cancer cell lines (HT 29 and LoVo) were less sensitive with IC₅₀ values of 26 and 12 μM, respectively. The colon cancer cell line HT 29 was insensitive to EP-302. The nucleolin surface negative cell lines NIH 3T3 or normal breast epithelial cells (MCF-10A were insensitive to EP-302 with IC₅₀ values >100 (6 H) and 53 μM after 24 h incubation. These results show that EP-302 has activity in cancer cell lines that express cell surface nucleolin.

TABLE 3
Screening of various cancer cell lines and a normal mouse
fibroblast cell line (NIH-3T3) and normal breast
epithelial (MCF-10A) cell lines for cytotoxicity of EP-302.

| | EP-302 | EP-302 |
|---|---|---|
| | (IC₅₀ [μM]) | (IC₅₀ [μM]) |
| Cell Line | 6 h | 24 h |
| MDA-MB-435S (Breast) | 1.4 ± 0.09 | 1.7 ± 0.2 |
| MDA-MB-231 (Breast) | 4.6 ± 0.9 | 3.6 ± 0.4 |
| MCF-7 (Breast) | 5.5 ± 0.4 | 6.4 ± 0.9 |
| OVCAR-3 (Ovary) | 5.2 ± 0.3 | ND |
| SKOV-3 (Ovary) | 5.1 ± 0.2 | 5.2 ± 0.9 |
| CHO (Hamster Ovary) | 3.1 ± 0.9 | 3.7 ± 0.2 |
| PC-3 (Prostate) | 3.3 ± 0.3 | 3.2 ± 0.8 |
| DU145 (Prostate) | 3.1 ± 0.02 | 5.1 ± 0.1 |
| MiaPaCa (Pancreas) | 2.1 ± 0.2 | 1.9 ± 0.1 |
| LoVo (Colon) | 14.1 ± 0.5 | 12.5 ± 0.3 |
| HT 29 (Colon) | 45.8 ± 0.4 | 26.2 ± 2.2 |
| HT 116(Colon) | 6.7 ± 0.9 | 4.8 ± 1.1 |
| A549 (Lung) | 4.3 ± 0.6 | 2.4 ± 0.08 |
| HL60 (Acute Myeloid Leukemia | ND | 0.5 |
| MV4-11(Acute Myeloid Leukemia) | ND | 0.5 |
| K562 (Chronic Myeloid Leukemia) | ND | 2.8 |
| Daudi (NHL) | 3.2 ± 0.4 | 2.5 ± 0.05 |
| MCF-10A (normal breast cells) | ND | 68 |
| NIH-3T3 (mouse fibroblast cells) | 257 ± 130 | 53 ± 9.1 |

ND = Not Determined

### Example 7

This example provides data showing the binding specificity of peptide constructs.

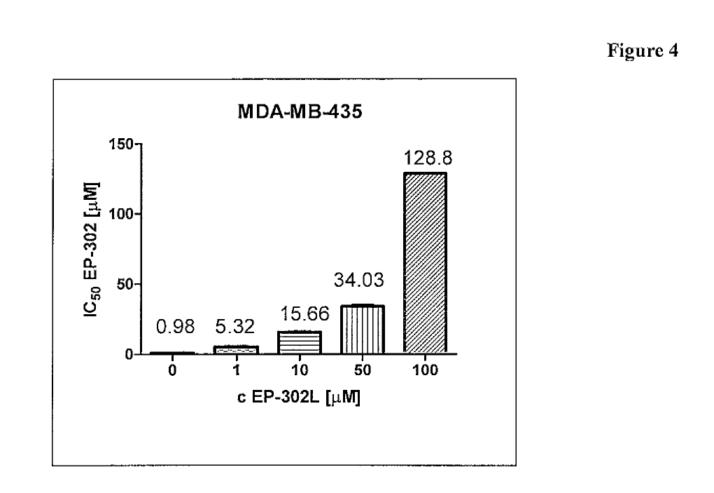

In vitro specificity was studied by pre-treatment of human breast cancer cell line MDA-MB-435S by adding increasing concentrations (0, 1, 10, 50 and 10 μM) of D-enantiomer of the nucleolin-binding peptide (R₁ARLQRRSARLSAK) to the cell culture medium, followed by addition of EP-302 (0.0001, 0.01, 0.1, 1 5, 10, 50, 100 μM). Results (FIG. 4) showed that increasing concentrations of the peptide decreased cytotoxicity of EP-302 in a concentration dependent manner indicating that EP-302 targets nucleolin as the binding site for its cytotoxic activity.

### Example 8

This example provides data measuring hemolytic activity of peptide constructs.

Hemolytic activity of the compounds was studied by incubating EP-301 and EP-302 with human red blood cells at concentrations of 0.01, 0.1, 1, 10 and 50 μM for 2 hours. Results (FIG. 3B) show that both EP-301 and EP-302 were not hemolytic.

### Example 9

This example includes studies showing in viva tolerability of peptide constructs.

In vivo tolerability studies were conducted in female nude mice (5 mice per group) that received for 5 consecutive days 5 and 10 mg/kg doses of intravenously injected EP-302 (dissolved in saline at dose concentrations of 1.25 and 2.5 mg/ml). Body weight, blood chemistry and blood cell counts were measured. All mice survived the repeated injections (Table 4) with no signs of toxicity indicating that EP-302 is well tolerated. No adverse observations were made during necropsy, body weight remained stable, no necrosis on tails observed. Blood parameters for CBC and serum chemistry were normal in treated mice of both groups.

TABLE 4
Summary of Survival Study BalbC/nude mice, repeat dose

| | | | | | Survival | Survival |
|---|---|---|---|---|---|---|
| | Mice per | Survival | Survival | Survival | [%] | [%] |
| EP-302 | Group | [%] Day 1 | [%] Day 2* | [%] Day 3 | Day 4 | Day 5 |
| 5 mg/kg | 5 | 100 (5/5) | 100 (5/5) | 100 (5/5) | 100 (5/5) | 100 (5/5) |
| 10 mg/kg | 5 | 100 (5/5) | 100 (5/5) | 100 (5/5) | 100 (5/5) | 100 (5/5) |

### Example 10

This example includes in-vivo studies showing the effect of peptides and peptide constructs on tumor size and animal survival rates.

The effect of multiple doses of the fusion construct EP-302 or unconjugated peptide (D-enantiomer of RARLQRRSARLSAK) were studied in male nude mice bearing human PC-3 prostate cancer and female nude mice implanted with human MDA-MB-435S breast cancer cells. Tumors were induced in nude mice by subcutaneous injections of the human cancer cells. The tumors were allowed to grow before injecting the mice with the peptides. EP-302 was dissolved in saline and administered intravenously, slowly through the lateral tail vein of mice (N=16 per group) bearing PC-3 xenografts at 0.02, 0.2 and 1mg/kg on Days 17, 21, 25 and 30 after inoculation of the cells. The unconjugated peptide D-amino acid RARLQRRSARLSAK was given at 1 mg/kg. Control animals received saline. MDA-MB-435S breast cancer bearing mice (10 mice per group) were treated intravenously with saline (controls), EP-302 (0.2 mg/kg) or RARLQRRSARLSAK (1 mg/kg). Three-dimensional measurements of tumors were made by caliper and were used to calculate tumor volume.

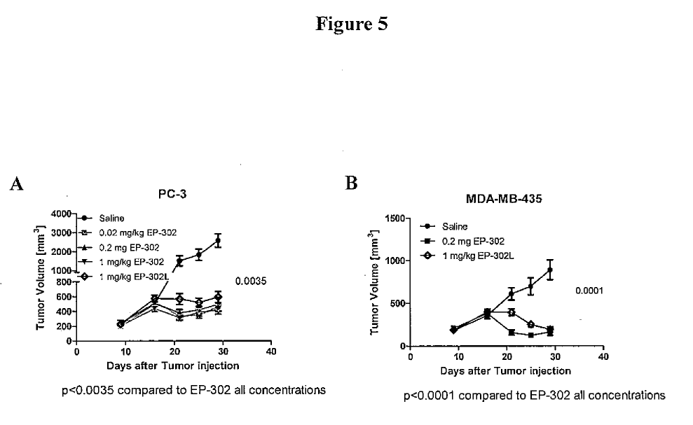

Results (FIG. 5) show that EP-302 reduced tumor volume in PC-3 xenografts and in the MDA-MB-435S xenograft models. EP-302 increased survival in the PC-3 xenograft model (75% death in saline control). Injection of D-amino acid RARLQRRSARLSAK peptide caused tumor volume arrest in the PC-3 xenograft and a reduction of tumor volume after the 3^{rd} injection in the MDA-MB-435s xenograft. Again, the D-amino acid RARLQRRSARLSAK peptide increased the survival of PC-3 xenograft bearing mice compared to saline controls, indicating that this peptide had anti-tumor activity in vivo.

### Example 11

This example includes studies showing the effect of lytic peptide conjugated to an anti-nucleolin antibody.

Cytotoxicity of an antibody to nucleolin conjugated to lytic peptide, Phor-18 (KFAKFAK KFAKFAK KFAK) was measured in various human cancer cell lines. The cell lines expressed surface nucleolin, and were human pancreatic cancer cell line, MiaPaCa, breast cancer cell line, MDA-MB-435S, acute leukemia cell line, HL60,T-cell leukemia, Jurkat. A mouse fibroblast cell line (3T3) that does not express surface nucleolin, was used as a negative control.

Chemical conjugation to lytic peptide: Purified mouse anti-nucleolin monoclonal antibody (MS-3, IgG₁) was purchased from Santa Cruz Biotechnology, Santa Cruz, Calif. reconstituted in phosphate buffered saline (PBS) to a concentration of approximately 2 mg/ml. A 20 mM solution of N-succinidyl-3-(2-pyridylothio)propionate (SPDP) was freshly prepared in dimethyl sulfoxide, and added to the antibody solution in 20-fold molar excess. The mixture was incubated at room temperature for about 30 minutes to produce the antibody-linker intermediate. Excess unreacted SPDP was removed by size exclusion chromatography. The cytotoxic molecule containing cysteine was thoroughly reduced by reaction with a 10-fold molar excess of reductaryl reagent before mixing in 10-fold excess with the antibody-linker construct. The reaction was allowed to incubate at room temperature for 18 hours and desalted to remove unreacted molecules. The solution was sterilized before storage.

In vitro cytotoxicity studies: In vitro toxicity studies were conducted using surface nucleolin positive cells MiaPaCa (human pancreatic cancer cell line), MDA-MB-435S (human breast cancer cell line), HL60 (human AML cell line), Jurkat (human T cell leukemia cell line) and a surface nucleolin negative mouse fibroblast cell line (3T3) to determine the toxicity of the anti-nucleolin antibody-Phor18 (KFAKFAKKFAKFAKKFAK) conjugate (ADC). Cell cultures from the nucleolin positive cell line MDA-MB-435S, MiaPaCa, HL60, U897, and Jurkat and the surface nucleolin negative cell line 3T3 were prepared in 96 well plates using 5,000 for adherent cultures (MDA-MB-435S, MiaPaCa and 3T3) and 15,000 cells/well for suspension cells (HL60, U937 and Jurkat). Adherent cells were allowed to attach for 48 hours.

The ADC was diluted in saline and added to cells at increasing concentrations of 0, 0.0015, 0.015, 0.15, 1.5, 15, 150, 300 and 500 nM. Incubations were conducted for 15 h at 37° C. Cell viability was determined using a formazan conversion assay (MTT assay). Controls contained USP saline or 0.1% TritonX-100™ as reference for 0 and 100% cell death, respectively. Data were processed and analyzed using Graph Pad Prizm 4™ software (Graph Pad Prizm, Inc).

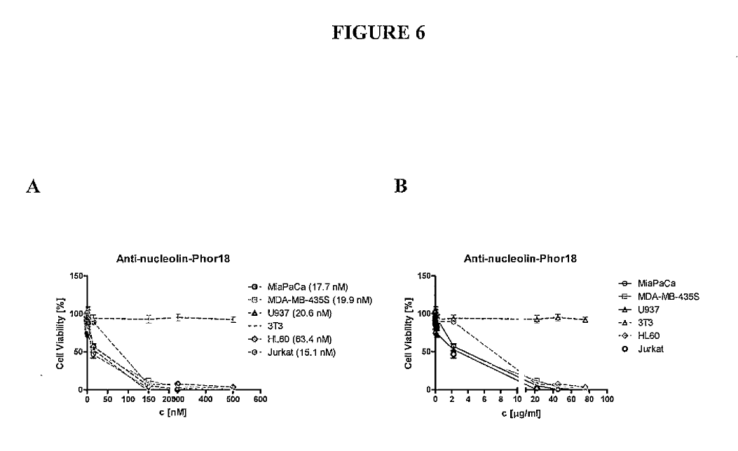

As shown in Table 5 and FIG. 6, the anti-nucleolin antibody-Phor 18 conjugate (ADC) was strongly cytotoxic towards cells that express nucleolin on their surfaces: the human breast cancer cell line MDA-MB-435S, MiaPaCa, HL60, U937 and Jurkat cells with IC₅₀ values [nM] for ADC were 17.7±5.9, 19.9±4.8, 63.42±3.8, 20.67±3.8, 15.06±4.2 nM, respectively, after 15 h of incubation. The 3T3 cell line was not killed (IC₅₀=60,860±15,160 nM) indicating that the effects of ADC were mediated via surface nucleolin since only surface nucleolin positive cells responded.

TABLE 5
Cytotoxicity of Phor18-anti-nucleolin antibody drug conjugates (ADC)
to Cells (nanomolar IC₅₀) after 15 hr incubation.

| | ADC |
|---|---|
| Cell Line | [IC₅₀ nM] |
| MDA-MB-435S | 17.71 ± 5.9 |
| MiaPaCa | 19.95 ± 4.8 |
| U937 | 20.67 ± 3.8 |
| HL60 | 63.4 ± 3.7 |
| Jurkat | 15.08 ± 4.3 |
| 3T3 | 60,860 ± 15,160 |

The results indicate that anti nucleolin antibody-Phor-18 conjugates linked through chemical synthesis are active in the nanomolar range against surface nucleolin presenting cell lines.

### Example 12

This example includes a description of additional in vivo studies.

Chemically linked anti-nucleolin antibody (mouse anti-nucleolin monoclonal antibody) is chemically conjugated to Phor18 (KFAKFAKKFAKFAKKFAK) as described previously. Recombinant antibody or antibody fragment conjugated to Phor18 is produced in mammalian, yeast or bacterial systems and purified by standard procedures (such as hydrophobic interaction chromatography followed by ion exchange or affinity chromatography). Recombinant antibodies or antibody fragments include but are not limited to scFv-Phor18, scFv-C_{H}2-C_{H}3-Phor18, scFv-C_{H}3-Phor18, scFv-GS-Phor18, scFv-C_{H}2-CH_{H}3-GS-Phor18, scFv-C_{H}3-GS-Phor18, scFv-NRVRRS-Phor18, scFv-C_{H}3-NRVRRS-Phor18, scFv-C_{H}2-C_{H}3NRVRRS-Phor18, scFv-(Phor18)₂, scFv-C_{H}2-C_{H}3-(Phor18)₂, scFv-C_{H}3-(Phor18)₂. The Phor18 moiety can be linked to the N or C terminal or to both terminals.

Chemically linked whole antibody Phor18 conjugates are linked through various linkers, such as peptide linkers (e.g., GS, AF, FK, VK, FFK, FA, GSGRSA, RVRRSV, SS, Citruline-Valine, F-Citrulline), at various lengths. Thioether, N-succinidyl-3-(2-pyridylothio)propionate, thioether bonds such as SLAB [N-succinimidyl (4-iodoacetyl) aminobenzoate], SMCC [succinimidyl-4-(N-maleimidomethyl)cyclohexane-1-carboxylate], MBS (m-Maleimidobenzoyl-N-hydroxysuccinimide) and SMPB [succinimidyl-4-(p-maleimidophenyl)butyrate], maleimide or hydrazone linkers may be used to conjugate Phor18 to the antibody or antibody fragment.

Xenograft tumor mouse models are used to analyze in vivo anti-tumor activity of the conjugated antibody or antibody fragments. For example, female Nu/Nu mice are injected subcutaneously with a MDA-MB-435S/Matrigel suspension (1×10⁶ cells). The mice (12 per group) are randomly allocated to groups and treated with saline (N=12) (controls), naked anti-nucleolin antibody (5 mg/kg) (N=12), anti-nucleolin-Phor18 conjugate (ADC) (0.5 mg/kg) (N=12) or ADC (5 mg/kg) (N=12) on day 21 after tumor cell injection on tumors of median tumor volume of 100±20 mm³ and continued on days 28, and 36 as a bolus intravenous injection into the lateral tail vein. During the entire study tumor volumes and body weights are measured twice weekly. Final necropsy is conducted on day 42 after tumor cell injection where tumors are excised, weighed and fixed in formalin for histological evaluation.

1. An isolated peptide comprising an amino acid sequence X1-X2-X3-X4-X5-X6-X7-X8-X9-X10-X11-X12-X13-X14, wherein:
X1 is R, H, G, K or V;
X2 is A, G or V;
X3 is R, K or H;
X4 is L;
X5 is Q;
X6 is R;
X7 is R;
X8 is S, F or L;
X9 is A;
X10 is R;
X11 is L or nothing;
X12 is S, F or L or nothing;
X13 is A or nothing;
X14 is K or nothing; and
wherein the peptide binds nucleolin.

2. The peptide of claim 1, wherein the peptide comprises the sequence:
RARLQRRSARLSAK, KARLQRRSARLSAK, VARLQRRSARLSAK,
HARLQRRSARLSAK, GARLQRRSARLSAK, RAKLQRRSARLSAK,
HAHLQRRSARLSAK, RARLQRRFARLFAK, KARLQRRFARLFAK,
VARLQRRFARLFAK, GARLQRRFARLFAK, HARLQRRFARLFAK,
HAHLQRRFARLFAK, RAKLQRRFARLFAK, RARLQRRLARLLAK,
KARLQRRLARLLAK, VARLQRRLARLLAK, GARLQRRLARLLAK,
HARLQRRLARLLAK, RAKLQRRLARLLAK, HAHLQRRLARLLAK,
RARLQRRSARLSA, KARLQRRSARLSA, VARLQRRSARLSA,
HARLQRRSARLSA, GARLQRRSARLSA, RAKLQRRSARLSA,
HAHLQRRSARLSA, RARLQRRFARLFA, KARLQRRFARLFA,
VARLQRRFARLFA, GARLQRRFARLFA, HARLQRRFARLFA,
HAHLQRRFARLFA, RAKLQRRFARLFA, RARLQRRLARLLA,
KARLQRRLARLLA, VARLQRRLARLLA, GARLQRRLARLLA,
HARLQRRLARLLA, RAKLQRRLARLLA, HAHLQRRLARLLA,
RARLQRRSAR, KARLQRRSAR, VARLQRRSAR, HARLQRRSARL,
GARLQRRSAR, RAKLQRRSAR, HAHLQRRSAR, RARLQRRSAR,
KARLQRRFAR, VARLQRRFAR, GARLQRRFARLFA,
HARLQRRFAR, HAHLQRRFAR, RAKLQRRFAR, RARLQRRLAR,
KARLQRRLAR, VARLQRRLAR, GARLQRRLAR, HARLQRRLAR,
RAKLQRRSAR, HAHLQRRLAR, RGRLQRRSARLSAK,
RVRLQRRSARLSAK, RGRLQRRSARLSA, RVRLQRRSARLSA,
RGRLQRRSARLS, RVRLQRRSARLS, RGRLQRRSARL,
RVRLQRRSARL, RGRLQRRSAR,
or
RVRLQRRSAR.

3. (canceled)

4. The peptide of claim 1, wherein the peptide has a length of 100 residues or less.

5.-7. (canceled)

8. The peptide of claim 1, wherein the peptide binds to a cell expressing nucleolin.

9. The peptide of claim 1, wherein the peptide is cytotoxic to cells expressing nucleolin.

10. The peptide of claim 1, wherein the peptide comprises or consists of one or more L- or D-amino acid residues

11. The peptide of claim 1, wherein the peptide further comprises or is conjugated to a lytic domain.

12. A fusion construct comprising or consisting of a peptide having an amino acid sequence: RARLQRRSARLSAK, KARLQRRSARLSAK, VARLQRRSARLSAK, HARLQRRSARLSAK, GARLQRRSARLSAK, RAKLQRRSARLSAK, HAHLQRRSARLSAK, RARLQRRFARLFAK, KARLQRRFARLFAK, VARLQRRFARLFAK, GARLQRRFARLFAK, HARLQRRFARLFAK, HAHLQRRFARLFAK, RAKLQRRFARLFAK, RARLQRRLARLLAK, KARLQRRLARLLAK, VARLQRRLARLLAK, GARLQRRLARLLAK, HARLQRRLARLLAK, RAKLQRRLARLLAK, HAHLQRRLARLLAK, RARLQRRSARLSA, KARLQRRSARLSA, VARLQRRSARLSA, HARLQRRSARLSA, GARLQRRSARLSA, RAKLQRRSARLSA, HAHLQRRSARLSA, RARLQRRFARLFA, KARLQRRFARLFA, VARLQRRFARLFA, GARLQRRFARLFA, HARLQRRFARLFA, HAHLQRRFARLFA, RAKLQRRFARLFA, RARLQRRLARLLA, KARLQRRLARLLA, VARLQRRLARLLA, GARLQRRLARLLA, HARLQRRLARLLA, RAKLQRRLARLLA, HAHLQRRLARLLA, RARLQRRSAR, KARLQRRSAR, VARLQRRSAR, HARLQRRSARL, GARLQRRSAR, RAKLQRRSAR, HAHLQRRSAR, RARLQRRFAR, KARLQRRFAR, VARLQRRFAR, GARLQRRFARLFA, HARLQRRFAR, HAHLQRRFAR, RAKLQRRFAR, RARLQRRLAR, KARLQRRLAR, VARLQRRLAR, GARLQRRLAR, HARLQRRLAR, RAKLQRRLAR, HAHLQRRLAR, RGRLQRRSARLSAK, RVRLQRRSARLSAK, RGRLQRRSARLSA, RVRLQRRSARLSA, RGRLQRRSARLS, RVRLQRRSARLS, RGRLQRRSARL, RVRLQRRSARL, RGRLQRRSAR, or RVRLQRRSAR, and a lytic domain, wherein the fusion construct binds to nucleolin.

13. The peptide of claim 11, or fusion construct of claim 12, wherein the lytic domain is positioned at the amino or carboxy terminus of the peptide.

14. The peptide of claim 11, or fusion construct of claim 12, wherein the lytic domain is joined to the peptide by a covalent bond, or a peptide or non-peptide linker.

15. The peptide of claim 11, or fusion construct of claim 12, wherein the lytic domain comprises or consists of an amino acid sequence selected from KFAKFAKKFAKFAK (Phor14), KFAKFAKKFAKFAKK, KFAKFAKKFAKFAKKF, KFAKFAKKFAKFAKKFA, KFAKFAKKFAKFAKKFAK, KFAKFAKKFAKFAKKFAKF, KFAKFAKKFAKFAKKFAKFA and KFAKFAKKFAKFAKKFAKFAK (Phor21); or an amino acid sequence selected from KFAKFAKKFAKFAK (Phor14), KFAKFAKKFAKFAKK, KFAKFAKKFAKFAKKF, KFAKFAKKFAKFAKKFA, KFAKFAKKFAKFAKKFAK, KFAKFAKKFAKFAKKFAKF, KFAKFAKKFAKFAKKFAKFA and KFAKFAKKFAKFAKKFAKFAK (Phor21) having one or more of the K residues substituted with any of an F or L residue, one or more of the F residues substituted with any of a K, A or L residue, or one or more of the A residues substituted with any of a K, F or L residue.

16. (canceled)

17. The peptide of claim 13, wherein the peptide or lytic domain forms an alpha helix.

18. The peptide of claim 13, wherein the peptide or lytic domain is cationic.

19. The peptide of claims 13, wherein the peptide or lytic domain is amphipathic.

20. A fusion construct comprising an antibody or antibody fragment thereof that binds to nucleolin and a lytic domain, wherein the lytic domain comprises or consists of an amino acid sequence selected from KFAKFAKKFAKFAK (Phor14), KFAKFAKKFAKFAKK, KFAKFAKKFAKFAKKF, KFAKFAKKFAKFAKKFA, KFAKFAKKFAKFAKKFAK, KFAKFAKKFAKFAKKFAKF, KFAKFAKKFAKFAKKFAKFA and KFAKFAKKFAKFAKKFAKFAK (Phor21); or an amino acid sequence selected from KFAKFAKKFAKFAK (Phor14), KFAKFAKKFAKFAKK, KFAKFAKKFAKFAKKF, KFAKFAKKFAKFAKKFA, KFAKFAKKFAKFAKKFAK, KFAKFAKKFAKFAKKFAKF, KFAKFAKKFAKFAKKFAKFA and KFAKFAKKFAKFAKKFAKFAK (Phor21) having one or more of the K residues substituted with any of an F or L residue, one or more of the F residues substituted with any of a K, A or L residue, or one or more of the A residues substituted with any of a K, F or L residue.

21. (canceled)

22. The fusion construct of claim 20, wherein said antibody fragment comprises an Fab, Fab′, F(ab′)₂, Fv, Fd, single-chain Fv (scFv), disulfide-linked Fvs (sdFv), V_{L}, V_{H}, Camel Ig, V-NAR, VHH, trispecific (Fab₃), bispecific (Fab₂), diabody ((V_{L}-V_{H})₂ or (V_{H}-V_{L})₂), triabody (trivalent), tetrabody (tetravalent), minibody ((scFᵥ-C_{H}3)₂), bispecific single-chain Fv (Bis-scFv), IgGdeltaCH2, scFv-Fc, (scFv)₂-Fc, affibody, aptamer, avimer or nanobody.

23. The peptide of claim 1, or fusion construct of claim 12, wherein the peptide or fusion construct further comprises a cytotoxic moiety.

24. (canceled)

25. The peptide of claim 1, or fusion construct or lytic peptide of claim 12, wherein the peptide or fusion construct or lytic domain comprises a continuous amphipathic alpha helical structure of at least 30% of the length of the peptide.

26. (canceled)

27. The peptide of claim 1, or fusion construct or lytic peptide of claim 12, wherein the peptide or fusion construct or lytic domain has no detectable hemolytic activity against human red blood cells.

28. The peptide of claim 1, or fusion construct or lytic peptide of claim 12, wherein the peptide or fusion construct or lytic domain has a hemolytic activity of more than about 500 μM, or more than about 250 μM, or more than about 100 μM, or more than about 75 μM, or more than about 50 μM, or more than about 25 μM, or more than about 10 μM, or more than about 5 μM, against human red blood cells.

29.-33. (canceled)

34. A pharmaceutical composition comprising the peptide of claim 1, or fusion construct of claim 12.

35.-39. (canceled)

40. A method of reducing or inhibiting cell proliferation, comprising contacting a nucleolin expressing cell with the peptide of claim 1, or fusion construct of claim 12, in an amount sufficient to reduce or inhibit proliferation of the nucleolin expressing cell.

41. (canceled)

42. A method of treating a subject having a neoplasia, tumor, cancer or malignancy, comprising administering to the subject an amount of the peptide of claim 1, or fusion construct of claim 12, sufficient to reduce or inhibit proliferation of the neoplasia, tumor, cancer or malignancy.

43.-70. (canceled)

71. A method of reducing or inhibiting angiogenesis, comprising contacting a cell with the peptide of claim 1, or fusion construct of claim 12, in an amount sufficient to reduce or inhibit angiogenesis.

72.-78. (canceled)
